(19) United States
(12) Patent Application Publication
(10) Pub. No.: US 2016/0000866 A1
(43) Pub. Date: Jan. 7, 2016

(54) CTHRC1 RECEPTOR AND METHODS OF USE THEREOF

(71) Applicant: MAINE MEDICAL CENTER, Scarborough, ME (US)

(72) Inventors: Volkhard Lindner, South Portland, ME (US); Calvin P. Vary, Windham, ME (US); Julia P. Stohn, Biddeford, ME (US)

(21) Appl. No.: 14/789,479

(22) Filed: Jul. 1, 2015

Related U.S. Application Data
(60) Provisional application No. 62/019,949, filed on Jul. 2, 2014.

(51) Int. Cl. A61K 38/17; G01N 33/68

(52) U.S. Cl. CPC A61K 38/1709; G01N 33/6887; G01N 2333/4704; G01N 2800/10

(57) ABSTRACT

The invention features compositions and methods for treating and preventing steatosis, osteoporosis, and muscle weakness featuring the receptor for the collagen triple helix repeat containing-1 (Cthrc1) protein.

### RELATED APPLICATIONS

This application claims the benefit of priority under 35 U.S.C. §119(e) to U.S. Provisional Application No. 62/019,949, filed Jul. 2, 2014, which is incorporated herein by reference in its entirety.

### STATEMENT OF RIGHTS TO INVENTIONS MADE UNDER FEDERALLY SPONSORED RESEARCH

This work was supported by The National Institutes of Health under grant number HL69182. The government has certain rights in the invention.

### FIELD OF THE INVENTION

This invention relates generally to the field of metabolism.

### BACKGROUND OF THE INVENTION

CTHRC1 is a circulating factor expressed at sites of tissue injury and remodeling. Prior to the invention described herein, the CTHRC1 receptor had not been identified. As such, there is a pressing need to identify the CTHRC1 receptor for the treatment of various conditions in which CTHRC1 plays a role.

### SUMMARY OF THE INVENTION

The invention is based, at least in part, on the surprising discovery that G Protein-Coupled Receptor 180 (GPR180) is a receptor for CTHRC1. GPR180 (also known as intimal thickness-related receptor (ITR)) encodes a protein that is a member of the G protein-coupled receptor superfamily, which is expressed in many tissues including the liver.

Provided herein are methods of treating or preventing steatosis or a steatosis-associated condition in a subject by identifying a subject having or at risk of developing steatosis or a steatosis-associated condition and administering to the subject an effective amount of a Collagen Triple Helix Repeat Containing 1 (CTHRC1) polypeptide or a CTHRC1 receptor agonist, thereby treating or preventing steatosis or a steatosis-associated condition in the subject. For example, the steatosis or a steatosis-associated condition is selected from the group consisting of hepatic steatosis and cardiac steatosis. The composition is administered orally, intravenously, intramuscularly, or systemically. Preferably, the effective amount of Cthrc1 polypeptide or Cthrc1 receptor agonist is sufficient to reduce lipids in target organs by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. For example, the Cthrc1 receptor comprises G Protein-Coupled Receptor 180 (GPR180).

Methods of treating or preventing low bone mass or a low bone mass-associated condition in a subject are carried out by identifying a subject having or at risk of developing low bone mass or a low bone mass-associated condition and administering to the subject an effective amount of a CTHRC1 polypeptide or a CTHRC1 receptor agonist, thereby treating or preventing low bone mass or a low bone mass-associated condition in the subject. For example, the low bone mass or low bone mass-associated condition comprises osteoporosis. The composition is administered orally, intravenously, intramuscularly, or systemically. The effective amount of CTHRC1 receptor agonist is sufficient to increase bone mass by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. For example, the CTHRC1 receptor comprises GPR180.

Also provided are methods of treating or preventing muscle weakness or a muscle weakness-associated condition in a subject by identifying a subject having or at risk of developing muscle weakness or a muscle weakness-associated condition; and administering to the subject an effective amount of a Cthrc1 polypeptide or a Cthrc1 receptor agonist, thereby treating or preventing muscle weakness or a muscle weakness-associated condition in the subject. For example, the muscle weakness or a muscle weakness-associated condition comprises muscular dystrophy or age-related frailty and muscle weakness. The muscle weakness or a muscle weakness-associated condition also includes any non-pathological condition where increased strength is desireable, e.g., physical activity, including athletic activity.

The composition is administered orally, intravenously, intramuscularly, or systemically. The effective amount of Cthrc1 receptor agonist is sufficient to increase muscle strength by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. For example, the CTHRC1 receptor comprises GPR180.

Methods for identifying a subject having low bone bass are carried out by obtaining a biological sample from said subject, determining the level of a CTHRC1 polypeptide in a biological sample from the subject, comparing the level of a CTHRC1 polypeptide in a biological sample from the subject to a normal control level of a CTHRC1 polypeptide, wherein a reduced level of CTHRC1 relative to the normal control level is indicative of the subject having low bone mass. In some cases, a reduced level of CTHRC1 relative to the normal control level is at least 1% reduced as compared to the normal control, e.g., at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% reduced as compared to the normal control.

The invention also provides methods for identifying a subject having muscle weakness, the method comprising obtaining a biological sample from said subject, determining the level of a CTHRC1 polypeptide in a biological sample from the subject, and comparing the level of a CTHRC1 polypeptide in a biological sample from the subject to a normal control level of a CTHRC1 polypeptide, wherein a reduced level of CTHRC1 relative to the normal control level is indicative of the subject having muscle weakness. For example, the muscle weakness comprises reduced strength of fast-twitch muscle fibers. In some cases, a reduced level of CTHRC1 relative to the normal control level is at least 1% reduced as compared to the normal control, e.g., at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% reduced as compared to the normal control.

Also provided are methods of treating or preventing metabolic syndrome or a metabolic syndrome-associated condition in a subject by identifying a subject having or at risk of developing metabolic syndrome or a metabolic syndrome-associated condition and administering to the subject an effective amount of a CTHRC1 polypeptide or a CTHRC1 receptor agonist, thereby treating or preventing metabolic syndrome or a metabolic syndrome-associated condition in the subject. The CTHRC1 receptor comprises a G Protein-Coupled Receptor 180 (GPR180).

Exemplary effective doses of CTHRC1 polypeptide or a CTHRC1 receptor agonist include between 0.1 μg/kg and 100 mg/kg body weight, e.g., 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, or 900 μg/kg body weight or 1, 2, 3, 4, 5, 6, 7, 8, 9, 10, 20, 30, 40, 50, 60, 70, 80, 90, or 100 mg/kg body weight.

In some cases, the CTHRC1 polypeptide or a CTHRC1 receptor agonist is administered daily, e.g., every 24 hours. Or, the CTHRC1 polypeptide or a CTHRC1 receptor agonist is administered continuously or several times per day, e.g., every 1 hour, every 2 hours, every 3 hours, every 4 hours, every 5 hours, every 6 hours, every 7 hours, every 8 hours, every 9 hours, every 10 hours, every 11 hours, or every 12 hours.

Exemplary effective daily doses of CTHRC1 polypeptide or a CTHRC1 receptor agonist include between 0.1 μg/kg and 100 μg/kg body weight, e.g., 0.1, 0.3, 0.5, 1, 5, 10, 15, 20, 25, 30, 35, 40, 45, 50, 55, 60, 65, 70, 75, 80, 85, 90, 95, or 99 μg/kg body weight.

Alternatively, the CTHRC1 polypeptide or a CTHRC1 receptor agonist is administered about once per week, e.g., about once every 7 days. Or, the CTHRC1 polypeptide or a CTHRC1 receptor agonist is administered twice per week, three times per week, four times per week, five times per week, six times per week, or seven times per week. Exemplary effective weekly doses of CTHRC1 polypeptide or a CTHRC1 receptor agonist include between 0.0001 mg/kg and 4 mg/kg body weight, e.g., 0.001, 0.003, 0.005, 0.01. 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, or 4 mg/kg body weight. For example, an effective weekly dose of CTHRC1 polypeptide or a CTHRC1 receptor agonist is between 0.1 μg/kg body weight and 400 μg/kg body weight.

Ultimately, the attending physician or veterinarian decides the appropriate amount and dosage regimen.

Methods of treating or preventing atherosclerosis or an atherosclerosis-associated condition in a subject are carried out by identifying a subject having or at risk of developing atherosclerosis or an atherosclerosis-associated condition and administering to the subject an effective amount of a Collagen Triple Helix Repeat Containing 1 (CTHRC1) polypeptide antagonist or a CTHRC1 receptor antagonist, thereby treating or preventing steatosis or a steatosis-associated condition in the subject. For example, the CTHRC1 receptor comprises G Protein-Coupled Receptor 180 (GPR180).

Also provided are methods of treating or preventing obesity or an obesity-associated condition in a subject by identifying a subject having or at risk of developing atherosclerosis or an atherosclerosis-associated condition and administering to the subject an effective amount of a Collagen Triple Helix Repeat Containing 1 (CTHRC1) polypeptide antagonist or a Cthrc1 receptor antagonist, thereby treating or preventing obesity or an obesity-associated condition in the subject. For example, the Cthrc1 receptor comprises G Protein-Coupled Receptor 180 (GPR180). In one aspect, the obesity-associated condition is diabetes.

The subject is preferably a mammal in need of such treatment, e.g., a subject that has been diagnosed with diabetes or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

Exemplary effective doses of CTHRC1 polypeptide antagonist or a CTHRC1 receptor antagonist include between 0.1 μg/kg and 100 mg/kg body weight, e.g., 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, or 900 μg/kg body weight or 1, 2, 3, 4, 5, 6, 7, 8, 9, 10, 20, 30, 40, 50, 60, 70, 80, 90, or 100 mg/kg body weight.

In some cases, the CTHRC1 polypeptide antagonist or a CTHRC1 receptor antagonist is administered daily, e.g., every 24 hours. Or, the CTHRC1 polypeptide antagonist or a CTHRC1 receptor antagonist is administered continuously or several times per day, e.g., every 1 hour, every 2 hours, every 3 hours, every 4 hours, every 5 hours, every 6 hours, every 7 hours, every 8 hours, every 9 hours, every 10 hours, every 11 hours, or every 12 hours.

Exemplary effective daily doses of CTHRC1 polypeptide antagonist or a CTHRC1 receptor antagonist include between 0.1 μg/kg and 100 μg/kg body weight, e.g., 0.1, 0.3, 0.5, 1, 5, 10, 15, 20, 25, 30, 35, 40, 45, 50, 55, 60, 65, 70, 75, 80, 85, 90, 95, or 99 μg/kg body weight.

Alternatively, the CTHRC1 polypeptide antagonist or a CTHRC1 receptor antagonist is administered about once per week, e.g., about once every 7 days. Or, the CTHRC1 polypeptide antagonist or a CTHRC1 receptor antagonist is administered twice per week, three times per week, four times per week, five times per week, six times per week, or seven times per week. Exemplary effective weekly doses of CTHRC1 polypeptide antagonist or a CTHRC1 receptor antagonist include between 0.0001 mg/kg and 4 mg/kg body weight, e.g., 0.001, 0.003, 0.005, 0.01. 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, or 4 mg/kg body weight. For example, an effective weekly dose of CTHRC1 polypeptide antagonist or a CTHRC1 receptor antagonist is between 0.1 μg/kg body weight and 400 μg/kg body weight.

Ultimately, the attending physician or veterinarian decides the appropriate amount and dosage regimen.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

Provided herein are CTHRC1 polypeptide agonists and antagonists, as well as CTHRC1 receptor agonists and antagonists (i.e., GPR180 agonists and antagonists).

The term “agonist” as used herein, refers to any molecule which enhances the biological activity of its target molecule.

As used herein, the terms “antagonist” and “inhibitor” are used interchangeably to refer to any molecule that counteracts or inhibits, decreases, or suppresses the biological activity of its target molecule. Suitable CTHRC1 polypeptide antagonists include soluble receptors (e.g., soluble CTHRC1 receptor, i.e., GPR180), peptide inhibitors, small molecule inhibitors, ligand fusions, and antibodies.

The term “receptor antagonist,” as used herein, refers to an agent that is capable of specifically binding and inhibiting signaling through a receptor to fully block or detectably inhibit a response mediated by the receptor.

The agonists or antagonists may include but are not limited to nucleic acids, peptides, antibodies, or small molecules that bind to their specified target or the target's natural ligand and modulate the biological activity.

Provided herein are methods for screening CTHRC1 polypeptide agonists and antagonists, as well as CTHRC1 receptor agonists and antagonists (i.e., GPR180 agonists and antagonists) for desired biological activity. For example, a CTHRC1 polypeptide agonist is screened to confirm it enhances the biological activity of a CTHRC1 polypeptide, while a CTHRC1 receptor agonist is screened to confirm that it enhances the biological activity of a CTHRC1 receptor. Similarly, a CTHRC1 polypeptide antagonist is screened to confirm that it counteracts or inhibits, decreases, or suppresses the biological activity of a CTHRC1 polypeptide, while a CTHRC1 receptor antagonist is screened to confirm that it counteracts or inhibits, decreases, or suppresses the biological activity of a CTHRC1 receptor.

In some cases, nucleic acids, e.g., ribonucleic acid (RNA) or deoxyribonucleic acid (DNA), inhibit the expression of CTHRC1 polypeptide or CTHRC1 receptor, thereby inhibiting the activity of CTHRC1 or CTHRC1 receptor. In some cases, the nucleic acid comprises small interfering RNA (siRNA), RNA interference (RNAi), messenger RNA (mRNA), short hairpin RNA (shRNA), or microRNA. Thus, suitable CTHRC1 antagonists include CTHRC1 siRNA and CTHRC1 shRNA, each of which is available from Santa Cruz Biotechnology, Inc., Dallas, Tex. and incorporated herein by reference.

For example, provided herein are small molecule agonists and small molecule antagonists, i.e., inhibitors. A small molecule is a compound that is less than 2000 Daltons in mass. The molecular mass of the small molecule is preferably less than 1000 Daltons, more preferably less than 600 Daltons, e.g., the compound is less than 500 Daltons, less than 400 Daltons, less than 300 Daltons, less than 200 Daltons, or less than 100 Daltons. Small molecules are organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecules can be synthetically engineered to consist of a fragment, or small portion, or a longer amino acid chain to fill a binding pocket of an enzyme. Typically, small molecules are less than one kiloDalton.

Described herein are anti-CTHRC1 antibodies. For example, monoclonal antibodies 10G07 (Duarte et al., 2014 PLOS ONE, 9(6): e100449, incorporated herein by reference), 13D11, and 19C07 are specific for the N terminus of human CTHRC1 and do not react with rat or murine CTHRC1. Anti-CTHRC1 antibody, clone 13E09, recognizes an epitope located within the N terminal half of the molecule of both human and rodent CTHRC1. Also provided is anti-CTHRC1 antibody, H-213, incorporated herein by reference and anti-CTHRC1 antibody, T-19, which is incorporated herein by reference (Santa Cruz Biotechnology, Inc., Dallas, Tex.). Also included are the following anti-CTHRC1 antibodies: SAB1102667, HPA059806, SAB2107469, and SAB1402656, each of which is incorporated herein by reference (Sigma-Aldrich®, St. Louis, Mo.). Also included is the following anti-CTHRC1 antibody PA5-38054, incorporated herein by reference (Thermo Scientific, Waltham, Mass.). In some cases, the anti-CTHRC1 antibodies described herein are administered at a concentration of 0.1 μg/ml to 500 mg/ml.

Also provided are anti-CTHRC1 receptor antibodies, i.e., anti-GPR180 antibodies. For example, provided herein are the following anti-GPR180 antibodies: HPA047250, SAB4500617, SAB1303667, SAB1408931, each of which is incorporated herein by reference (Sigma-Aldrich®, St. Louis, Mo.). Also described herein is the following anti-GRP180 antibody: PA5-26788, incorporated herein by reference (Thermo Scientific, Waltham, Mass.). Also provided is an anti-GPR180 antibody, NBP2-14068, incorporated herein by reference (Novus Biologicals, Littleton, Colo.). Described herein is an anti-GPR180 antibody, ABIN952608, incorporated herein by reference (antibodies-online.com; Atlanta, Ga.). In some cases, the anti-GPR180 antibodies described herein are administered at a concentration of 0.1 μg/ml to 500 mg/ml.

Antibodies and fragments thereof described herein include, but are not limited to, polyclonal, monoclonal, chimeric, dAb (domain antibody), single chain, Fab, Fab′ and F(ab′)2 fragments, Fv, scFvs. A fragment of an antibody possess the immunological activity of its respective antibody. In some embodiments, a fragment of an antibody contains 1500 or less, 1250 of less, 1000 or less, 900 or less, 800 or less, 700 or less, 600 or less, 500 or less, 400 or less, 300 or less, 200 or less amino acids. For example, a protein or peptide inhibitor contains 1500 or less, 1250 of less, 1000 or less, 900 or less, 800 or less, 700 or less, 600 or less, 500 or less, 400 or less, 300 or less, 200 or less, 100 or less, 80 or less, 70 or less, 60 or less, 50 or less, 40 or less, 30 or less, 25 or less, 20 or less, 10 or less amino acids. For example, a nucleic acid inhibitor of the invention contains 400 or less, 300 or less, 200 or less, 150 or less, 100 or less, 90 or less, 80 or less, 70 or less, 60 or less, 50 or less, 40 or less, 35 or less, 30 or less, 28 or less, 26 or less, 24 or less, 22 or less, 20 or less, 18 or less, 16 or less, 14 or less, 12 or less, 10 or less nucleotides.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa (κ) and lambda (λ), based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha (α), delta (δ), epsilon (ε), gamma (γ) and mu (μ), respectively. The γ and α classes are further divided into subclasses on the basis of relatively minor differences in C_{H} sequence and function, e.g., humans express the following subclasses: IgG1, IgG2, IgG3, IgG4, IgA1, and IgA2. The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. e al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{H} when numbered in accordance with AHo; Honneger, A. and Plunkthun, A. J. Mol. Biol. 309:657-670 (2001)).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Also provided are variable domain antigen-binding sequences derived from human antibodies. Accordingly, chimeric antibodies of primary interest herein include antibodies having one or more human antigen binding sequences (e.g., CDRs) and containing one or more sequences derived from a non-human antibody, e.g., an FR or C region sequence. In addition, chimeric antibodies of primary interest herein include those comprising a human variable domain antigen binding sequence of one antibody class or subclass and another sequence, e.g., FR or C region sequence, derived from another antibody class or subclass. Chimeric antibodies of interest herein also include those containing variable domain antigen-binding sequences related to those described herein or derived from a different species, such as a non-human primate (e.g., Old World Monkey, Ape, etc). Chimeric antibodies also include primatized and humanized antibodies.

Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

A “humanized antibody” is generally considered to be a human antibody that has one or more amino acid residues introduced into it from a source that is non-human. These non-human amino acid residues are often referred to as “import” residues, which are typically taken from an “import” variable domain. Humanization is traditionally performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting import hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species.

A “human antibody” is an antibody containing only sequences present in an antibody naturally produced by a human. However, as used herein, human antibodies may comprise residues or modifications not found in a naturally occurring human antibody, including those modifications and variant sequences described herein. These are typically made to further refine or enhance antibody performance.

An “intact” antibody is one that comprises an antigen-binding site as well as a C_{L} and at least heavy chain constant domains, C_{H} 1, C_{H} 2 and C_{H} 3. The constant domains may be native sequence constant domains (e.g., human native sequence constant domains) or amino acid sequence variant thereof. Preferably, the intact antibody has one or more effector functions.

An “antibody fragment” comprises a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870; Zapata et al., Protein Eng. 8(10): 1057-1062 [1995]); single-chain antibody molecules; and multispecific antibodies formed from antibody fragments.

The phrase “functional fragment or analog” of an antibody is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, Fc_{ε}RI.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H} 1). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

The “Fc” fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

As used herein, an antibody that “internalizes” is one that is taken up by (i.e., enters) the cell upon binding to an antigen on a mammalian cell (e.g., a cell surface polypeptide or receptor). The internalizing antibody will of course include antibody fragments, human or chimeric antibody, and antibody conjugates. For certain therapeutic applications, internalization in vivo is contemplated. The number of antibody molecules internalized will be sufficient or adequate to kill a cell or inhibit its growth, especially an infected cell. Depending on the potency of the antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the infected cell.

As used herein, an antibody is said to be “immunospecific,” “specific for” or to “specifically bind” an antigen if it reacts at a detectable level with the antigen, preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. Affinity of an antibody for its cognate antigen is also commonly expressed as a dissociation constant K_{D}, and in certain embodiments, HuM2e antibody specifically binds to M2e if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M. Affinities of antibodies can be readily determined using conventional techniques, for example, those described by Scatchard et al. (Ann. N.Y. Acad. Sci. USA 51:660 (1949)).

Binding properties of an antibody to antigens, cells or tissues thereof may generally be determined and assessed using immunodetection methods including, for example, immunofluorescence-based assays, such as immuno-histochemistry (IHC) and/or fluorescence-activated cell sorting (FACS).

An antibody having a “biological characteristic” of a designated antibody is one that possesses one or more of the biological characteristics of that antibody which distinguish it from other antibodies. For example, in certain embodiments, an antibody with a biological characteristic of a designated antibody will bind the same epitope as that bound by the designated antibody and/or have a common effector function as the designated antibody.

The term “antagonist antibody” is used in the broadest sense, and includes an antibody that partially or fully blocks, inhibits, or neutralizes a biological activity of an epitope, polypeptide, or cell that it specifically binds. Methods for identifying antagonist antibodies may comprise contacting a polypeptide or cell specifically bound by a candidate antagonist antibody with the candidate antagonist antibody and measuring a detectable change in one or more biological activities normally associated with the polypeptide or cell.

Antibody “effector functions” refer to those biological activities attributable to the Fc region (a native sequence Fc region or amino acid sequence variant Fc region) of an antibody, and vary with the antibody isotype. Examples of antibody effector functions include: Clq binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

By “agent” is meant any small molecule chemical compound, antibody, nucleic acid molecule, or polypeptide, or fragments thereof.

By “Collagen Triple Helix Repeat Containing 1” or “Cthrc1” is meant a polypeptide having at least about 85%, e.g., at least about 90%, at least about 95%, or at least about 99%, sequence identity to NCBI Accession No. NP_AAQ89273, or a fragment thereof that regulates metabolism. An exemplary sequence of human CTHRC1 is (SEQ ID NO: 7):

| | |
|---|---|
| 1 | mrpqgpaasp qrlrglllll llqlpapssa seipkgkqka qlrqrevvdl yngmclqgpa |
| 61 | gvpgrdgspg anvipgtpgi pgrdgfkgek geclresfee swtpnykqcs wsslnygidl |
| 121 | gkiaectftk mrsnsalrvl fsgslrlkcr naccqrwyft fngaecsgpl pieaiiyldq |
| 181 | gspemnstin ihrtssvegl cegigaglvd vaiwvgtcsd ypkgdastgw nsysriiiee |
| 241 | lpk |

By a “nucleic acid encoding CTHRC1” is meant a nucleic acid having at least about 85%, e.g., at least about 90%, at least about 95%, or at least about 99%, sequence identity to NCBI Accession No. NM_{—}138455 or NM_{—}001256099. An exemplary nucleic acid encoding Cthrc1 is (SEQ ID NO: 8):

| | |
|---|---|
| 1 | gggagggaga gaggcgcgcg ggtgaaaggc gcattgatgc agcctgcggc ggcctcggag |
| 61 | cgcggcggag ccagacgctg accacgttcc tctcctcggt ctcctccgcc tccagctccg |
| 121 | cgctgcccgg cagccgggag ccatgcgacc ccagggcccc gccgcctccc cgcagcggct |
| 181 | ccgcggcctc ctgctgctcc tgctgctgca gctgcccgcg ccgtcgagcg cctctgagat |
| 241 | ccccaagggg aagcaaaagg cgcagctccg gcagagggag gtggtggacc tgtataatgg |
| 301 | aatgtgctta caagggccag caggagtgcc tggtcgagac gggagccctg gggccaatgg |
| 361 | cattccgggt acacctggga tcccaggtcg ggatggattc aaaggagaaa agggggaatg |
| 421 | tctgagggaa agctttgagg agtcctggac acccaactac aagcagtgtt catggagttc |
| 481 | attgaattat ggcatagatc ttgggaaaat tgcggagtgt acatttacaa agatgcgttc |
| 541 | aaatagtgct ctaagagttt tgttcagtgg ctcacttcgg ctaaaatgca gaaatgcatg |
| 601 | ctgtcagcgt tggtatttca cattcaatgg agctgaatgt tcaggacctc ttcccattga |
| 661 | agctataatt tatttggacc aaggaagccc tgaaatgaat tcaacaatta atattcatcg |
| 721 | cacttcttct gtggaaggac tttgtgaagg aattggtgct ggattagtgg atgttgctat |
| 781 | ctgggttggt acttgttcag attacccaaa aggagatgct tctactggat ggaattcagt |
| 841 | ttctcgcatc attattgaag aactaccaaa ataaatgctt taattttcat ttgctacctc |
| 901 | tttttttatt atgccttgga atggttcact taaatgacat tttaaataag tttatgtata |
| 961 | catctgaatg aaaagcaaag ctaaatatgt ttacagacca aagtgtgatt tcacactgtt |
| 1021 | tttaaatcta gcattattca ttttgcttca atcaaaagtg gtttcaatat tttttttagt |
| 1081 | tggttagaat actttcttca tagtcacatt ctctcaacct ataatttgga atattgttgt |
| 1141 | ggtcttttgt tttttctctt agtatagcat ttttaaaaaa atataaaagc taccaatctt |
| 1201 | tgtacaattt gtaaatgtta agaatttttt ttatatctgt taaataaaaa ttatttccaa |
| 1261 | caaccttaat atctttaaa |

Another exemplary nucleic acid encoding CTHRC1 is (SEQ ID NO: 9):

| | |
|---|---|
| 1 | agaaggttta aggccggaaa gggaaatgaa ggggcccggc gctaaccctc taaggacctg |
| 61 | ttttgcttct gtttaaacca aatgggcagt ctgtcattac acacaccctg ggtcttcata |
| 121 | tgtggccgcc aggtaggagc atcacagtca agctacggga gaaaacagtt tccaggaaac |
| 181 | tggaaatgaa cggcccgagt gctttccagg ggctcatctg tgggaagtat aatggaatgt |
| 241 | gcttacaagg gccagcagga gtgcctggtc gagacgggag ccctggggcc aatggcattc |
| 301 | cgggtacacc tgggatccca ggtcgggatg gattcaaagg agaaaagggg gaatgtctga |
| 361 | gggaaagctt tgaggagtcc tggacaccca actacaagca gtgttcatgg agttcattga |
| 421 | attatggcat agatcttggg aaaattgcgg agtgtacatt tacaaagatg cgttcaaata |
| 481 | gtgctctaag agttttgttc agtggctcac ttcggctaaa atgcagaaat gcatgctgtc |
| 541 | agcgttggta tttcacattc aatggagctg aatgttcagg acctcttccc attgaagcta |
| 601 | taatttattt ggaccaagga agccctgaaa tgaattcaac aattaatatt catcgcactt |
| 661 | cttctgtgga aggactttgt gaaggaattg gtgctggatt agtggatgtt gctatctggg |
| 721 | ttggtacttg ttcagattac ccaaaaggag atgcttctac tggatggaat tcagtttctc |
| 781 | gcatcattat tgaagaacta ccaaaataaa tgctttaatt ttcatttgct acctcttttt |
| 841 | ttattatgcc ttggaatggt tcacttaaat gacattttaa ataagtttat gtatacatct |
| 901 | gaatgaaaag caaagctaaa tatgtttaca gaccaaagtg tgatttcaca ctgtttttaa |
| 961 | atctagcatt attcattttg cttcaatcaa aagtggtttc aatatttttt ttagttggtt |
| 1021 | agaatacttt cttcatagtc acattctctc aacctataat ttggaatatt gttgtggtct |
| 1081 | tttgtttttt ctcttagtat agcattttta aaaaaatata aaagctacca atctttgtac |
| 1141 | aatttgtaaa tgttaagaat tttttttata tctgttaaat aaaaattatt tccaacaacc |
| 1201 | ttaatatctt taaa |

An exemplary nucleic acid sequence of murine GPR180 (GenBank Accession No.: NM_{—}021434.5 (GI:225579050), incorporated herein by reference) is (SEQ ID NO: 10):

| | |
|---|---|
| 1 | ctgctgccga cgtgggctgc aggccgcagg cggttgccgg gcgagcaaac ggagcgggcg |
| 61 | gcgggcatgg gcggcctgcg gctgctggcg gtagccctca cgtgcagctg ctggtggccg |
| 121 | cagggcggcc agggcaagac cctgcgtggc agcttcagca gcgccgcggc ccgcgacgcc |
| 181 | cagggccaga gcatcggcca tttcgagttc cacggtgacc atgctcttct gtgtgtcaga |
| 241 | atcaacaacg tagcagtggc tgttggaaaa gaagctaaac tctacctgtt ccaagcccag |
| 301 | gaatggctga agctgctgga gagcagcccc ggctacagct gcagtgagcg gctagcccga |
| 361 | gctcagctga cagtgacagt gacccagacg gagcacaacc tcacagtgtc ccagctgccc |
| 421 | gctccccaga catggcgagt gttctatgcc gacaagttca cctgcaggga tgactcagag |
| 481 | agcccccagg gggaggagat cccctttgaa atggtgctcc tcaacccgga cgccgaggga |
| 541 | aacccactgg atcattttag cgccagagag tccgggctcc acgagttctt tttcctcctc |
| 601 | gtcctagtgt actttgtgac tgcgtgcatc tatgcgcagt ctctgtggca ggctatgaag |
| 661 | aagggaggac ccatgcacac catcttaaag gtcctcacca ctgcactgct gcttcaagct |
| 721 | gcttcagcct tagctaatta catccacttg tccaggtact ccagagatgg gctaggagtg |
| 781 | cctctcatag gaagcctggc agaagttttt gacattgcct cccaaattca gatgctgtac |
| 841 | ctgcttctga gcctgtgtat gggctggaca atagtgcgga tgaagaagtc gcagagcaga |
| 901 | ccgctccagt gggactcgac acccgcgtcc acgggcatcg cagtcttcat cgtcatcaca |
| 961 | cagagcattt tgctactctg ggagcagttt gaagacacca gtcaccacag cgcacattca |
| 1021 | caccgcagct tagccgggct cttgctgatt gtcttacgga tctgcctggc gctgtcgctg |
| 1081 | ggctgcggac tctaccaggt catcacagtg gagaggagcg cgctcaagag agagttctac |
| 1141 | atcacgtttg ccaagggctg catcctgtgg ttcttgtgcc agccagcgct cgcatgcatt |
| 1201 | gctgtcgctt ttaatgacta ccaaagagat aagcttatca cagtaggtgt catcctgtgt |
| 1261 | caggccgtgg ccatggtcat tctgtacaga cttttcctgt cccacagtct ttactgggag |
| 1321 | gtctcctcgc tctcctcagt aacgctacca ctgaccatct cgtctgcaca cagagggcgc |
| 1381 | cctcatttct gatgcttgag ttttgtggag agaaccagtg aatggagaag tgcaatagga |
| 1441 | tccaacgcag caccgtcttg ctgtgccttt gtgtgacagc tgagcggtgg aagcagggcg |
| 1501 | tcttatttat agaactgaac gtcagcgggc tcagcagaaa ggaatagaag ctccggagtg |
| 1561 | aactcaaaca gtgaacttcc cagaaagaat gttgtttcaa ggtgactgaa acagtttcca |
| 1621 | cgtggaaata aatgtgaaaa ggactgctta gagtacacgt gggccaggtg gtcacacctg |
| 1681 | cgatgcctcg tcactagcaa actcaggcct gatagtccta cagtattcac ctagacaata |
| 1741 | cttgcctgtg cgtgcccagc tcgcccagtt atgaaatcag cgggatgtgc tgattttaaa |
| 1801 | actacttctt tttatccttt aaagaacgtg catttcaaat tataatttaa aggacttgaa |
| 1861 | agtgaaatta cttaggaaat aaatagaaaa tatgttaaca gttaaacgaa ttagcatttg |
| 1921 | tttattcacg tgtggagtta ttctgcaatt tctccttgta ttgttggccc tgcttcaggt |
| 1981 | cttatcttga ttgaggaagg atcttggata aagtgtggaa aatcatgggt acattacatt |
| 2041 | ggttggaaga acagagagct taagtgtgag gacgtgggta agggacaagc agggttttgt |
| 2101 | gtttaaaaag aaagaaggaa gaggtagtat gtggtatggc cctgaaattt taatcctgtg |
| 2161 | ataattcatt tctcttgcgc ggaatcccca gtcctaatga agctgtagac acagattaaa |
| 2221 | ttaacaggag catttcacag gactgttagt ttccgtcatc aacctaccca cgaattcctg |
| 2281 | cccaaggata cttaatcagg attaaatttt ctatgaataa ttgaaaaaca aaatactcac |
| 2341 | tggatttgtt ctaatccatg ttggcactgg atggtaagga gttgccatct tgaatccttt |
| 2401 | gtaataaagc tgtgtgtgtg tgtgtttgtg tgtgtgtgtg tgtgtgtgtg tgtgtgtgtg |
| 2461 | tgtgtgtgtt tgtgtgtgtg tgtgtgtgtg ttgctcccca aattaagtat gctagtcctc |
| 2521 | taaaaggttc ctcattaaag aaccactctg tagctgtcag gacttgctgt gtgctggtat |
| 2581 | ctgtgaagtc gcttggacct tattgtttac ttttaactaa ttttaaatat ttaatctgct |
| 2641 | tgccatattt ttaaacagga aatggtgcaa taatctggta attgcgtttt ttaatggaag |
| 2701 | ggttcagtta atgtagcaga atggaaaagg actgtggctt tgtctttatt ttttccttgt |
| 2761 | ctatagtttc attgaaaatg aaaatatcct gcttcatttt aagacaaaat gtgtttgcag |
| 2821 | tcagcaaaat ggcaaagtaa tacagtctag cccgtgatta aaacatttct ataggtt |

An exemplary amino acid sequence of murine GPR180 (GenBank Accession No.: EDL00568.1 (GI:148668238), incorporated herein by reference) is (SEQ ID NO: 11):

| | |
|---|---|
| 1 | mgglrllava ltcscwwpqg gqgktlrgsf ssaaardaqg qsighfefhg dhallcvrin |
| 61 | nvavavgkea klylfqaqew lkllesspgy scserlaraq ltvtvtqteh nitvsqlpap |
| 121 | qtwrvfyadk ftcrddsesp qgeeipfemv llnpdaegnp ldhfsaresg lhefffllvl |
| 181 | vyfvtaciya qslwqamkkg gpmhtilkvl ttalllqaas alanyihlsr ysrdglgvpl |
| 241 | igslaevfdi asqiqmlyll lslcmgwtiv rmkksqsrpl qwdstpastg iavfivitqs |
| 301 | illlweqfed tshhsahshr slaglllivl riclalslgc glyqvitver salkrefyit |
| 361 | fakgcilwfl cqpalaciav afndyqrdkl itvgvilcqa vamvilyrlf lshslywevs |
| 421 | slssvtlplt issahrgrph f |

An exemplary nucleic acid sequence of human GPR180 (GenBank Accession No.:

NM_{—}180989.5 (GI:324710989), incorporated herein by reference) is (SEQ ID NO: 12):

| | |
|---|---|
| 1 | ctcccccagc tgccgacgtg gggcgggcag ccgccggcgg ctgggagccg aggcgtcggt |
| 61 | gcagacctgg agacgggcat gggggggctg cggctgctgg ctgtggccct cacgtgctgc |
| 121 | tggtggccgc agggcagcca gggtaagacc ctgcggggca gcttcagcag caccgcggcc |
| 181 | caggacgccc agggccagcg catcggccac ttcgagttcc atggtgacca tgctcttctg |
| 241 | tgtgtcagaa tcaacaacat agcagtagct gttggaaaag aagctaaact ctacctgttc |
| 301 | caagcccagg aatggctaaa gctacagcaa agcagtcatg gttatagctg tagtgaaaaa |
| 361 | ttatccaaag ctcagttgac aatgaccatg aaccagaccg aacataatct gacagtgtcc |
| 421 | cagattccgt ctccacaaac gtggcatgtg ttttatgcag acaagtatac atgccaagat |
| 481 | gacaaggaga attctcaggt ggaagatatc ccatttgaaa tggtgttact aaacccagat |
| 541 | gccgaaggga atccatttga tcattttagt gctggagaat ctgggttaca tgagttcttt |
| 601 | ttcctcctag tcctagtgta ctttgtgatt gcttgcattt atgctcaatc attgtggcag |
| 661 | gctattaaga aaggcggacc catgcacatg attttaaagg ttctgacaac tgcattgctg |
| 721 | ttacaagctg gttcagcttt agctaattac attcatttct ccagttactc caaagatgga |
| 781 | ataggggtac catttatggg aagtttggca gaattttttg acatcgcttc ccaaattcag |
| 841 | atgttatact tacttttgag tctatgcatg ggttggacaa tagtcagaat gaagaagtct |
| 901 | caaagcagac ctctccagtg ggattctacg cctgcatcca ctggcattgc agtattcatt |
| 961 | gtcatgacac agagtgtttt gctactttgg gaacagtttg aagatatcag tcatcatagc |
| 1021 | taccattcac accacaactt agcagggatc ctcctaattg ttctaagaat ttgcctagca |
| 1081 | ttgtcattag gctgtggact ctatcagatc atcacagtgg agagaagtac actcaaaagg |
| 1141 | gagttctaca tcacatttgc caaaggctgt atcttgtggt ttttatgcca tccagttctt |
| 1201 | gcatgcattt ctgtcatttt tagcgactac caaagagaca aggttattac aataggtgtt |
| 1261 | atcctttgcc agtctgtttc catggttatt ctctacagac tctttctgtc tcacagtcta |
| 1321 | tactgggaag tttcttcact ttcttcagta acactaccac tgaccatatc atctggacac |
| 1381 | aaaagtcgcc ctcatttctg atacttgatt tttgttgaga ggaaaagtga attggttaaa |
| 1441 | agagtgcaat aaggatccaa atacagtgac ttttttttca tacatttagt atgaaaactt |
| 1501 | gaacagcgaa agcagagcat gttatttata taactgcatt taagcagtac caagactgaa |
| 1561 | aaaaaaggta ataaatgaaa tgttttgaaa tatacttaaa caacaaactt tgaagaaagt |
| 1621 | gttgttataa aattattgaa gcgatttcta tgtggaaata aatgtgaaaa ataactatga |
| 1681 | tattttggta aaatattcac cacttataat gcctcatctt aatagctaac tcaggtttaa |
| 1741 | tagtcttata aaaagtaatc agttaaatga ttacttgctt ataaatatct aaactagtcc |
| 1801 | agttatgaaa tcagtgtaat acattgattt ttaaaactgc tgctttttat gctttaagga |
| 1861 | aaatgtattt catatttgag tttaaaggaa ttgaaattac ttcaggaaat gaatataaaa |
| 1921 | taggttcaca gttaaatgaa taagcttttg tttatttgtg ggtggagtta ttctccaatt |
| 1981 | ttttctgcca tttttggctc tagttcaggt tttagcttga ttagcaaagg tttttgacaa |
| 2041 | acagtttatg aaaaaataaa acttaaatac attacacggg ttgtaaggac aaaggatttt |
| 2101 | aaaatctgag cacttaggtg aagggacaag caggtttatg tgtttaaaca gaaagaaggg |
| 2161 | aaaaggtact atgtgatatg gtactgaaat tttgatccca atagaattca tttctcttac |
| 2221 | gttgaatccc caatcataat taagccgtat acacagatta aattaacaga agcatttcac |
| 2281 | ataaatgttg gtttcagtca tcaactaccc atgaattcct gcccaaggat acttaatcag |
| 2341 | gattaaattt tctatgaata attgaaaaac aaaacactca ctggatttgt tataatccgt |
| 2401 | gttggcactg gattctaagt agttgccatc ttgaatcctt tgtaataaag ccatactttt |
| 2461 | gtgtgtgtgt gtgatgcccc agtattgagt atgctagcta aaaaatatca agtgcctgat |
| 2521 | taaagaattg ctaaatggtt gtcaatactc gctgtataaa atgagtattc ccattaatag |
| 2581 | tcactgattg gaaattattc tgttgctgtt tgtcaagctg ttcaggcctt ttccttttta |
| 2641 | cttctggctt attttaaaaa tattttttat ctatttactg tgtgtttaag caagcaacag |
| 2701 | tgcactgatc tggtagttaa attttttaat taaatagtta aaataatgta gtgcaactta |
| 2761 | aaaatatcta tggctttgtt tttgtttttc ttttgtacct agtctcattg gaaatgacta |
| 2821 | gtattctgcc tcattttcag ggcagaatat gtgtttgtga cccaaaatga caaagttacc |
| 2881 | aagttcaacc tgctcattaa atcatctctc tattagttct ttgtaaatca cttagtacat |
| 2941 | tagagctaca ggttaagact agaaagtctg agttatgttt taggtataca tgatcgtctg |
| 3001 | agtggtcata accttttctt ttatcatgtc tcgctaataa ccccagctat tgtctgttgt |
| 3061 | gttcagctga gatgcaaaaa agaaattaag caaaaaagaa aaagatgaag acttttcatg |
| 3121 | aattttgtga acttgccaaa aggggaaggg aaaaatcttt gtgttacttc actcaaagaa |
| 3181 | cataagtaac tgcaattaac atatatgaaa tttattactc tgcttgcatt tatgaaacta |
| 3241 | accagttttt taaactttaa ttcttaaatt tatggttgga aatgctgata atttattttg |
| 3301 | ttatttaaga gctgttgtta agtggaggaa agtagttgtt aataaatgta taaaactgtt |
| 3361 | cttgactagt aatcagggac aaaatttata gttcataagt catgacacag tattcgctct |
| 3421 | ttttctgaat gtttacatag agattcatca ctgcagatta cagaaagtta agtgtatact |
| 3481 | acaaactcta attaaagata aaaatctttt ctacttttct ttgtcagata atgctttttt |
| 3541 | atgtgttttt acattttttg aaagaagata aatggttcat ccagagcttt attaagaagc |
| 3601 | atttcttttc ttcccttaaa aaatagatgc ttatttttat tgacatgtgt ataataagat |
| 3661 | gggtgcctac tgtgatgcat tttaccaggc attttacctt cattattact catctctatt |
| 3721 | gtgataacca ggctcccatt taactgagta taagagagaa taaatgattt ctctaaggtc |
| 3781 | atcaaactat ttagtttttc cactttacct tcatgattta aggaacatgt attagctgtg |
| 3841 | ttttagtgag aatgaattgc tatccatcat aatttttgct gttgatttga acattaaaac |
| 3901 | tggaattgtg ctaaaatcag aacatgcgct tgaatatctg taagacctca gcacacttca |
| 3961 | gtatttgaac ctggctttta accagacagt attactagca ggagagttaa tctaggcaga |
| 4021 | agagcttgct ttagggtaat tatttattat ctccagtaaa gtcacatctg atgaacatag |
| 4081 | gagatacaca aatctgagat catctctaaa ataatgagac tagaacctat ctatcaacat |
| 4141 | gaacatgggc tccttcagca tgtttcttct aggagactgc acgttctttg gaatttggaa |
| 4201 | atttggaatt atctgtgaaa cattttctta aatagcttct gtggtatcaa atcattgagg |
| 4261 | ttgttttttg tttgttttga gaattgatat gattgttaga agtagccaga tacgaagagg |
| 4321 | taatactgtt ttttattcca acatgtattt gtgaattgag actaaaataa tgagattggt |
| 4381 | tttcctctgt gatctgaaag tagtgtggta gaagcaccgt agcttgattt cttgcaatag |
| 4441 | ctctctaagt aatattacca tgtccacact tgggcagcct tctgcacagc tccaggggca |
| 4501 | tcatatgtgt tgtgctgtag gagtgccatg aagcctggag gtgaggaaag aagtgatcct |
| 4561 | gcccactagg gccattctcc acagtgtagc caaaacaatc tcttaaaaat ggaaatcatc |
| 4621 | tggatgatga gcatacagaa attctttgtg ctaatcttgt aatttttttg taagtttgaa |
| 4681 | attatttcaa aaagtttaaa aatcatataa atcagatcaa actactactt ccctgacaaa |
| 4741 | aaccctccat gtgcctctca ttgcacttag gaaaagaagt cctcactcat tactatgttc |
| 4801 | tgcaaggccc atatgatctg gttatctctt aatttgtacc actctctcac acacatatcc |
| 4861 | cagcctcatt gtccttcatc aaaaaaagat ctagcttata tcacaccctc agatacttgg |
| 4921 | cactagccat cttctctgcc tgggacattc tcttccaaat cttagcatgg ttggaaggct |
| 4981 | gaatcctcat ccttcaggtt ctcagctgag atgtctttta gagaactcac catctgtggt |
| 5041 | ggatttatat ggtgcagttt agtcaacctg gagataggtt ctgcagaatt ctcttccatg |
| 5101 | cataatttca ggacaatgta ggccataaga aatgtttgat acaagatata gaaggcagaa |
| 5161 | gtaaagtagc agccagatct gcattctttt tgtaagagga agggccagac aaggcacaag |
| 5221 | gcacaaggca ctgttgcagc ttatccacat tattgcttat ctgttggctt ctcttggcct |
| 5281 | gtgggacaac agccaggccc acaggaacac cagttcctgc cggaatggct ccttcagctt |
| 5341 | ctctggctcc tgggccaggt gtgcgtttta ctctgacaaa gggtgccagc ttctgctgga |
| 5401 | caactccatc attcacactg gaggattaga ggcagggaga gacctgtatg gcttccagtc |
| 5461 | catcctcaca agttccactc agttcccaca ggttctactc atcctagcaa gttccttgct |
| 5521 | tcccccattt cccatgtctt ccctttccaa ctgccagaca caaaagaaac cgcctcctat |
| 5581 | aatccctata ataagcccct tattttgaat attaagatat gtgtatgtgt attagatcct |
| 5641 | agtggttctt ttctgatcag accctgatta accataccat cctacctaat gctattcccc |
| 5701 | catcctagtt ctatcatatt atcccttttt ttgtttgttg tacttagctg gtttatcttt |
| 5761 | gtcatctctc aaattagaat ataaatttta agtgagtttg tctctattgt tcattgctgt |
| 5821 | atgcctaata ctaggacact ccctggtgca tactagatgc tcaaaaatat ttttgagtga |
| 5881 | atgaaggcaa tgctgaaaaa gttccaaatc tgtacttaac agtgtagtat taattatgta |
| 5941 | atagaagtac ccgtagtgac ttcaataata ccttgcctat tctttgttca attgcatgga |
| 6001 | acttggtagt gtgtaaaaat cttgactagt caagtagttg tcataaaaca tcagttacga |
| 6061 | gaggatttct caaattctca cagaggcagt ttctctttat tttatgtagt cctgtaatag |
| 6121 | gaccatgtgt ctggtttctg gcctgccaca accagaagca ccaatttaaa agagaagttt |
| 6181 | ctagtcagct agaggaggga atgttgacag tagtcagaag cagctgggaa aagatgggaa |
| 6241 | aactagaaat gacaataaca ctcaaattgc agcagcctgg ggtcatttag tgtaggtgaa |
| 6301 | agcagccctg tgttgtgtat ctcagtagac tcttaagaga tttgttgttc aatgtattca |
| 6361 | tgacagtgac tgatggtaat caagaaaata ttaagttata agttatgttc agtagcctca |
| 6421 | aagaagtatg cgaaaacctt ctaatgtttt cttcttgtta aacaggtaat atatgcacat |
| 6481 | tgtacaaaaa tcagaaagtt gaaaaaggtg gggaaaagaa gtccttctct cactcatttc |
| 6541 | ccttccctga atccaaactc tgttaccagt tatctttcta gaaatggccc catgcacata |
| 6601 | taaacatata tgaaacacaa attatagcat aacatacaga cttttgcaca ttcagcaatt |
| 6661 | tattctgtag ccttacctag aattgtccca tttttttcac caattgcata gaactccatt |
| 6721 | atatggatgt attttgattt tttcaaagaa tcagttctgt tactgcacgt tgttggtttt |
| 6781 | cagtcttgct aatacagaca aggctgaaat taagacagta atcaagaatt tgccaagact |
| 6841 | tccttccctc ctcccaaaat gcctccactc caggttccag gtctagatcc ttgggatttt |
| 6901 | ctaggtgatg gaagtatctt tgttattcat agtgagctct gatagttacg ctaaccagat |
| 6961 | gactcaaata ggggctagcc acaccagaag accaaccatt ggattagagg gttggagttt |
| 7021 | tgagtcatat gatattagcc caacctctga ggtggagaga ggggttagag atggagttca |
| 7081 | gctttatata gagattgagt caattaatca tgcctgtcat gaaaccccga gagaaactct |
| 7141 | ggtcagtgat gctcaagtga acttccctgg ttggcaatac tctggtgtgt tctaggaggg |
| 7201 | tgacatgtcc ctgagggcac agaaactttg tgtttgggat cctcttacac cttgctctat |
| 7261 | gtatgtcttc ttttggctgt tctgatttgt atccttttgc tataataaac tgtaatagta |
| 7321 | cgcatagcac tttgctgagt tctgtgagtt gttctattta attatagaac ctgaaggtat |
| 7381 | tgtggggacc cctgaacttg cagccagttg gtcagaagta aggatggtcc tgggaatgcc |
| 7441 | caaagttgct agtttctgaa gtgagggcag tcatatggag gactgggccc ttaatctgtg |
| 7501 | aagtttggct taacttcagg cagttggtgt cagaagccat tgcaaatatc ctgatgatcc |
| 7561 | gtgcaaaaca aaaacactta acagaaaatt acgttgtact ctctgtacgg ataattttcg |
| 7621 | gttaagtgtt tttgttttcc atggattgta ctagatatca atctgataaa taacttttat |
| 7681 | tcattatagt tttaatttgc atttctctta ttaatttttg ttttaaaacc attttctttc |
| 7741 | cagttctacg atctgttaac atatttttcc cacttttcta ttgggttggt tgtctttttc |
| 7801 | ttattgattt gttaaaactc tttataacag aaattggtca tttgtgatat gagtctgaac |
| 7861 | tgtgtttctc aatttgtgat ttttctcttt acttttttaa tggtgtcttt ttcttgtagt |
| 7921 | ttttttaatg caggtggaat tatcagtctt tcattttttt cttctgtgtt ctacctgttt |
| 7981 | cacaggtaga aaagccttag ctctttgaga ttatgtttaa atgccctgta ttttcatcta |
| 8041 | gtggttttat ggttttattt tgtatgtttt tttcatccac ctggaattta ttttgctgtt |
| 8101 | aaaatatgaa ttagagatcc aacttaattt ttttcagatg gttgttgact tattgcaata |
| 8161 | tcatgtgtta tataatccat ctttactgat tcgttgctaa ttggtttctg gacttttaag |
| 8221 | gctatctgat gaactgttga tctggtacca cactgtttca attattgtag tcttaaaatt |
| 8281 | gctgtacgct ttggtaggga taattttctg ttaagtgttt ttgtttttta taattttcca |
| 8341 | attagtcttg ctcatttttt atataaactt tgaaatcaat ttttctgatt ttcaaaagaa |
| 8401 | aaaaaatcct gtttagtatt cttagtggaa tgaattaaat ttccatagca atttagggaa |
| 8461 | atttgacttt cttaacgtgt tgagtgttaa acccacaggt ggtagcatga cattgactcc |
| 8521 | tcagccaagc atattatcaa ctgtcggaaa ctaggttttc ccatagtgat ccaagaaact |
| 8581 | agtacgaaat gcctttctga ttttttaaaa cttgtataca tatatagtac atattatata |
| 8641 | tacttatata ttaagtaatc agaagtatgt gtttctcaat caccttttaa agacttcttt |
| 8701 | atatagctct tgtacatttc ttgatatgaa tatgttaatg ttttctttca ttatgttgca |
| 8761 | tatggctttt gttttctgca tgaaaactat tgatttctgc attctaattt tgttcccagt |
| 8821 | cacctaactg aattcttcta acagttgtaa taaatttctg gttggctctc ttgtgaaaaa |
| 8881 | aaaaaaaaaa aaaaa |

An exemplary amino acid sequence of human GPR180 (GenBank Accession No.: AAH52243.1 (GI:30410915), incorporated herein by reference) is (SEQ ID NO: 13):

| | |
|---|---|
| 1 | mgglrllava ltccwwpqgs qgktlrgsfs staaqdaqgq righfefhgd hallcvrinn |
| 61 | iavavgkeak lylfqaqewl klqqsshgys cseklskaql tmtmnqtehn ltvsqipspq |
| 121 | twhvfyadky tcqddkensq vedipfemvl lnpdaegnpf dhfsagesgl hefffllvlv |
| 181 | yfviaciyaq slwqaikkgg pmhmilkvlt talllqagsa lanyihfssy skdgigvpfm |
| 241 | gslaeffdia sqiqmlylll slcmgwtivr mkksqsrplq wdstpastgi avfivmtqsv |
| 301 | lllwegfedi shhsyhshhn lagillivlr iclalslgcg lyqiitvers tlkrefyitf |
| 361 | akgcilwflc hpvlacisvi fsdyqrdkvi tigvilcqsv smvilyrlfl shslywevss |
| 421 | lssvtlplti ssghksrphf |

By “control” or “reference” is meant a standard of comparison. As used herein, “changed as compared to a control” sample or subject is understood as having a level of the analyte or diagnostic or therapeutic indicator to be detected at a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g, β-galactosidase or luciferase). Depending on the method used for detection, the amount and measurement of the change can vary. Determination of statistical significance is within the ability of those skilled in the art, e.g., the number of standard deviations from the mean that constitute a positive result.

As used herein, “detecting” and “detection” are understood that an assay performed for identification of a specific analyte in a sample, e.g., an antigen in a sample or the level of an antigen in a sample. The amount of analyte or activity detected in the sample can be none or below the level of detection of the assay or method.

By “diagnosing” as used herein refers to a clinical or other assessment of the condition of a subject based on observation, testing, or circumstances for identifying a subject having a disease, disorder, or condition based on the presence of at least one indicator, such as a sign or symptom of the disease, disorder, or condition. Typically, diagnosing using the method of the invention includes the observation of the subject for multiple indicators of the disease, disorder, or condition in conjunction with the methods provided herein. A diagnostic method provides an indicator that a disease is or is not present. A single diagnostic test typically does not provide a definitive conclusion regarding the disease state of the subject being tested.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “polynucleotide” or “nucleic acid” as used herein designates mRNA, RNA, cRNA, cDNA or DNA. As used herein, a “nucleic acid encoding a polypeptide” is understood as any possible nucleic acid that upon (transcription and) translation would result in a polypeptide of the desired sequence. The degeneracy of the nucleic acid code is well understood. Further, it is well known that various organisms have preferred codon usage, etc. Determination of a nucleic acid sequence to encode any polypeptide is well within the ability of those of skill in the art.

In some cases, a compound (e.g., small molecule) or macromolecule (e.g., nucleic acid, polypeptide, or protein) of the invention is purified and/or isolated. As used herein, an “isolated” or “purified” small molecule, nucleic acid molecule, polynucleotide, polypeptide, or protein (e.g., antibody or fragment thereof), is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA), e.g., synthetic cDNA) is free of the genes or sequences that flank it in its naturally occurring state. Purified also defines a degree of sterility that is safe for administration to a human subject, e.g., lacking infectious or toxic agents.

Thus, an “isolated” or “purified” polypeptide can be in a cell-free solution or placed in a different cellular environment (e.g., expressed in a heterologous cell type). The term “purified” does not imply that the polypeptide is the only polypeptide present, but that it is essentially free (about 90-95%, up to 99-100% pure) of cellular or organismal material naturally associated with it, and thus is distinguished from naturally occurring polypeptide. Similarly, an isolated nucleic acid is removed from its normal physiological environment. “Isolated” when used in reference to a cell means the cell is in culture (i.e., not in an animal), either cell culture or organ culture, of a primary cell or cell line. Cells can be isolated from a normal animal, a transgenic animal, an animal having spontaneously occurring genetic changes, and/or an animal having a genetic and/or induced disease or condition. An isolated virus or viral vector is a virus that is removed from the cells, typically in culture, in which the virus was produced.

By “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

As used herein, “kits” are understood to contain at least one non-standard laboratory reagent for use in the methods of the invention in appropriate packaging, optionally containing instructions for use. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100.mu·g/ml denatured salmon sperm DNA (ssDNA). In a most preferred embodiment, hybridization will occur at 42° C. C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42.degree. C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Obtaining” is understood herein as manufacturing, purchasing, or otherwise coming into possession of.

As used herein, “operably linked” is understood as joined, preferably by a covalent linkage, e.g., joining an amino-terminus of one peptide, e.g., expressing an enzyme, to a carboxy terminus of another peptide, e.g., expressing a signal sequence to target the protein to a specific cellular compartment; joining a promoter sequence with a protein coding sequence, in a manner that the two or more components that are operably linked either retain their original activity, or gain an activity upon joining such that the activity of the operably linked portions can be assayed and have detectable activity, e.g., enzymatic activity, protein expression activity.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like. Formulations of the present invention include those suitable for oral, nasal, topical, transdermal, buccal, sublingual, intramuscular, intracardiac, intraperotineal, intrathecal, intracranial, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, “plurality” is understood to mean more than one. For example, a plurality refers to at least two, three, four, five, or more.

A “polypeptide” or “peptide” as used herein is understood as two or more independently selected natural or non-natural amino acids joined by a covalent bond (e.g., a peptide bond). A peptide can include 2, 3, 4, 5, 6, 7, 8, 9, 10, 11, 12, 13, 14, 15, 16, 17, 18, 19, 20, or more natural or non-natural amino acids joined by peptide bonds. Polypeptides as described herein include full length proteins (e.g., fully processed proteins) as well as shorter amino acids sequences (e.g., fragments of naturally occurring proteins or synthetic polypeptide fragments). Optionally the peptide further includes one or more modifications such as modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins, Structure and Molecular Properties, 2nd ed., T. E. Creighton, W.H. Freeman and Company, New York (1993); Posttranslational Covalent Modification of Proteins, B. C. Johnson, ed., Academic Press, New York, pgs. 1-12 (1983); Seifter et al., Meth. Enzymol 182:626-646 (1990); Rattan et al., Ann. N.Y. Acad. Sci. 663:48-62 (1992)).

The term “reduce” or “increase” is meant to alter negatively or positively, respectively, by at least 5%. An alteration may be by 5%, 10%, 25%, 30%, 50%, 75%, or even by 100%.

A “sample” as used herein refers to a biological material that is isolated from its environment (e.g., blood or tissue from an animal, cells, or conditioned media from tissue culture) and is suspected of containing, or known to contain an analyte, such as a protein. A sample can also be a partially purified fraction of a tissue or bodily fluid. A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

A “subject” as used herein refers to an organism. In certain embodiments, the organism is an animal. In certain embodiments, the subject is a living organism. In certain embodiments, the subject is a cadaver organism. In certain preferred embodiments, the subject is a mammal, including, but not limited to, a human or non-human mammal. In certain embodiments, the subject is a domesticated mammal or a primate including a non-human primate. Examples of subjects include humans, monkeys, dogs, cats, mice, rats, cows, horses, goats, and sheep. A human subject may also be referred to as a patient.

A “subject sample” can be a sample obtained from any subject, typically a blood or serum sample, however the method contemplates the use of any body fluid or tissue from a subject. The sample may be obtained, for example, for diagnosis of a specific individual for the presence or absence of a particular disease or condition.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with diminished cardiac function is within the ability of those in the art. Subjects suffering from, and suspected of suffering from, a specific disease, condition, or syndrome are not necessarily two distinct groups.

As used herein, “susceptible to” or “prone to” or “predisposed to” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Ranges provided herein are understood to be shorthand for all of the values within the range. This includes all individual sequences when a range of SEQ ID NOs: is provided. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Unless specifically stated or obvious from context, as used herein, the term “or” is understood to be inclusive. Unless specifically stated or obvious from context, as used herein, the terms “a”, “an”, and “the” are understood to be singular or plural.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is a photograph illustrating validation of Cthrc1 monoclonal antibodies by Western blotting. The experiment was performed on conditioned medium (CM) or cell lysates (CL) of HEK293T cells and CHO-K1 cells transduced with an expression construct for human CTHRC1. The upper band of the two was identified as CTHRC1 with the signal peptide still attached.

FIG. 2 is a table showing basic patient characteristics of a sample of patients with and without detectable levels of Cthrc1. Summary statistics by site are shown as median (interquartile range) for continuous variables and number (percentage) for categorical variables. A missing value of Cthrc1 indicates that the ELISA could not be performed due to insufficient sample volume. Statistical tests involving Cthrc1 were performed with samples with nonmissing Cthrc1 readings; summary statistics for demographic samples were given for all samples.

FIG. 3 is a series of bone μCT images of femurs from 8 week old C57Bl/6J wildtype and Cthrc1 null mice on the same background. Note the reduction in trabecular bone.

FIG. 4A-FIG. 4F is a series of dot plots showing analyses of femurs from 25 week old Cthrc1 null mice and corresponding wildtype mice on the C57Bl/6J background, which was performed by μCT separately for males and females. FIG. 4A is a dot plot showing body weight. FIG. 4B is a dot plot showing relative trabecular bone volume. FIG. 4C is a dot plot showing relative trabecular bone volume. FIG. 4D is a dot plot showing trabecular thickness. FIG. 4E is a dot plot showing trabecular number. FIG. 4F is a dot plot showing trabecular separation. Trabecular bone volume and trabecular thickness were significantly reduced in Cthrc1 null mice.

FIG. 5A-FIG. 5C is a series of dot plots showing analyses of femurs from 25 week old Cthrc1 null mice and corresponding wildtype mice on the C57Bl/6J background, which was performed by μCT separately for males and females. FIG. 5A shows relative cortical bone volume. FIG. 5B shows cortical bone thickness. FIG. 5C shows cortical bone porosity. Cortical bone thickness was severely reduced in male Cthrc1 null mice but not females. Correspondingly, cortical bone porosity was significantly increased only in males.

FIG. 6A-FIG. 6B shows that grip strength was significantly reduced in both genders of Cthrc1 null mice. FIG. 6A is a dot plot showing grip strength in female Cthrc1 null mice. FIG. 6B is a dot plot showing grip strength in male Cthrc1 null mice.

FIG. 7A-FIG. 7F is a series of dot plots showing the results of myography, which was performed on the EDL muscle of Cthrc1 null and corresponding wildtype mice. FIG. 7A is a dot plot showing EDL mass. FIG. 7B is a dot plot showing EDL length. FIG. 7C is a dot plot showing EDL cross-sectional area. FIG. 7D is a dot plot showing EDL maximum twitch force. FIG. 7E is a dot plot showing EDL tetanic force. FIG. 7F is a dot plot showing EDL specific force.

FIG. 8A-FIG. 8C is a series of photomicrographs showing CTHRC1 expression in the neonatal vasculature and liver as target organ. FIG. 8A is a photomicrograph showing that CTHRC1 is expressed abundantly in outer mural cells of the remodeling aorta and pulmonary artery (PA) in newborn mice. FIG. 8B is a photomicrograph showing HRP activity as detected in liver. Recombinant Cthrc1 was injected into Cthrc1 null mice followed by injection of HRP conjugated, ELISA reactive anti-Cthrc1 10G7. Organs were harvested after extensive perfusion with PBS followed by detection of HRP activity with diaminobenzidine substrate on frozen sections. FIG. 8C is a photomicrograph showing that mice injected with 10G7-HRP alone (no Cthrc1) showed no HRP reactivity in the liver.

FIG. 9 is a photograph of a blot showing the expression analysis of G Protein-Coupled Receptor 180 (GPR180), which was performed for various mouse tissues using RT-PCR.

FIG. 10A-FIG. 10B is a series of photomicrographs showing the co-localization of GPR180 with bound Cthrc1. FIG. 10A is a series of photomicrographs showing that control transfected CHO-K1 cells did not bind any Cthrc1. FIG. 10B is a series of photomicrographs showing that binding of Cthrc1 was only observed in cells that also expressed GPR180. CHO-K1 cells were transfected with a myc-tagged GPR180 expression construct. Transfected cells were identified by immunocytochemistry with an anti-myc antibody (green). Cthrc1 bound to cells was identified by anti-Cthrc1 immunohistochemistry (red).

FIG. 11 is a schematic showing the function of Cthrc1, as identified herein. In healthy adults, low baseline levels of circulating Cthrc1 are maintained, influencing lipid metabolism via inhibition of Pparγ. Cthrc1 signals via its widely-expressed cognate receptor GPR180 to inhibit adipogenesis and lipogenesis. During inflammatory and stress conditions including tissue remodeling, pregnancy and diabetes, Cthrc1 levels are elevated, so as to conserve ATP levels via adenosine monophosphate-activated protein kinase (AMPK) activation. Cthrc1 deficiency causes increased FA oxidation, lowering of blood lipids with reduction in atherosclerosis.

FIG. 12A-FIG. 12D demonstrates validation of Cthrc1 specific monoclonal antibodies by Western blotting and ELISA. In FIG. 12A, Vli55 is a rabbit monoclonal raised against the conserved C terminal suitable for immunohistochemistry on paraffin sections. 10G7 and 08G09 are mouse monoclonal IgGs recognizing the N terminus of human or mouse/rat Cthrc1, respectively by indirect ELISA. These clones are used in the sandwich ELISA as biotin conjugates as detection antibodies. 13E9 is a mouse monoclonal IgG used as the capturing antibody in FIG. 12B, which demonstrates the results of the sandwich ELISA showing a standard curve established with purified Cthrc1. The analytical sensitivity of the assay was determined to be 75 pg/ml by calculating the mean±3 standard deviations for 16 replicates of the assay diluent. The lower limit of detection of the assay is 160 pg/ml. The lanes in FIG. 12A show conditioned medium (CM) and cell lysates from HEK293 and CHO cells transduced with Cthrc1 expressing adenovirus. CHO cells transduced with a Cthrc1 adenovirus show Cthrc1 in both CL and CM. Mass-spectrometry analysis revealed that the upper band is Cthrc1 that still has the signal peptide. Control adenovirus transduced CHO cells (Con CHO) show no endogenous Cthrc1. Please note that the entire blot is shown, not cropped bands. Cthrc1 plasma levels were monitored over a 24 h period and show increases after physical exercise (30 min of bicycling; FIG. 12C). FIG. 12 D shows a bar chart of diseases with significantly increased Cthrc1 levels, including inflammatory conditions and diabetes.

FIG. 13A-FIG. 13D is a series of photomicrographs demonstrating Cthrc1 is expressed in the pituitary, bone as well as remodeling tissues. Sections of a rat anterior pituitary (FIG. 13A), mouse femur (FIG. 13B), and mouse renal artery were immunostained for Cthrc1 expression (FIG. 13C and FIG. 13D). Rat pituitary shows Cthrc1 accumulations with expression by adjacent cells (arrows; FIG. 13A). Some osteocytes in adult bone show prominent expression with localization in canaliculi (FIG. 13B). In response to angiotensin II infusion a remodeling renal artery shows Cthrc1 expression in activated adventitial cells of wildtype mice undergoing fibrosis (FIG. 13C) and no Cthrc1 is detectable in Cthrc1 null mice infused with angiotensin II (FIG. 13D).

FIG. 14A-FIG. 14C is a photograph, dot plot, and chart showing that ApoE null mice without Cthrc1 are resistant to atherosclerosis. As shown in FIG. 14A, En face preparations of aortae were stained with oil red O to detect regions of lipid lesions. As shown in FIG. 14B, the percentage of lipid-covered area of the vessel was quantified between groups, and was significantly reduced on the Cthrc1 null background. As shown in FIG. 14C, preliminary blood chemistry analyses (Idexx Expanded Tox Panel) of pooled samples (n=6) from ApoE−/− and ApoE/Cthrc1 double null mice on a high fat diet revealed a less atherogenic lipid profile in the double mutant mice.

FIG. 15A-FIG. 15E is a series of photomicrographs showing evidence of the interaction between Gpr180 and Cthrc1. Tissues and cell cultures from various sources were used for RT-PCR to detect Grp180 mRNA (FIG. 15A). As shown in FIG. 15B, CHO-K1 cells do not express endogenous Gpr180. When these cells were treated with Cthrc1 protein and immunostained to detect Cthrc1 binding, no staining was observed. As shown in FIG. 15C, CHO-K1 were transfected with Gpr180-myc (green), and treated with Cthrc1 protein. The cells with expression of Grp180 bound to Cthrc1 protein, which was detected by immunostaining (red). In FIG. 15D, the results show that mice injected with anti-Cthrc1 antibody 10G07-HRP alone had no binding to the liver. FIG. 15E shows that following injection with Cthrc1 protein, 10G07-HRP interacted with the hepatocytes (brown reaction product).

FIG. 16A and FIG. 16B show the generation of Cre inducible Cthrc1 transgenic mice. As shown in FIG. 16A, the CAG-GFP-hCthrc1 construct was injected into oocytes from Cthrc1 null mice. FIG. 16B shows that breeding of Pdgfrb-Cre transgenic mice (Cuttler et al) results in vascular overexpression of the Cthrc1 transgene, which did not result in elevated circulating Cthrc1 levels in most mice examined. For generating mice with elevated circulating Cthrc1 levels the Tg(CAG-GFP-hCthrc1) mice are crossed with the tamoxifen inducible albumin-Cre mice (Albtm1(cre/ERT2)Mtz) (Schuler) now in the colony.

FIG. 17 A and FIG. 17B show that after normalizing energy expenditure to lean mass both at rest and under running wheel activity, Cthrc1 null mice show increased energy expenditure. As shown in FIG. 17C and FIG. 17D, the Seahorse analyzer was used to assess glycolytic function by measuring oxygen consumption rate (OCR) and extracellular acidification rate (ECAR) of C2C12 cells transduced with Cthrc1 (blue) or LacZ (red) expressing adenovirus. OCR is increased in Cthrc1 stimulated cells, suggestive of increase oxidative ATP synthesis. ECAR is increased in Cthrc1 stimulated cells following inhibition of oxidative phosphorylation by oligomycin (oligo) indicative of increased glycolytic capacity in response to Cthrc1 stimulation.

FIG. 18 is a photomicrograph showing stimulation of C2C12 cells (muscle precursor) with Cthrc1 for 2 hrs (100 ng/ml) increased phosphorylation of AMPK.

### DETAILED DESCRIPTION

The invention is based, at least in part, on the surprising discovery that G Protein-Coupled Receptor 180 (GPR180) is a receptor for Cthrc1. GPR180 encodes a protein that is a member of the G protein-coupled receptor superfamily, which protein is widely expressed.

The invention provides compositions featuring GPR180, GPR180 agonists, and GPR180 antagonists and methods for the use of such compositions for the treatment of various conditions in which CTHRC1 plays a role, e.g., metabolic syndrome, steatosis, low bone mass/osteoporosis, muscle weakness, etc. As described in detail herein, mass-spectrometry and co-localization studies were utilized to identify GPR180 as the receptor for CTHRC1. The GPR180 receptor is expressed in many organs (e.g., bone, muscle, and brain) and very prominently expressed in the liver. It has previously been reported that Cthrc1 null mice develop severe hepatic steatosis. As described in detail herein, Cthrc1 null mice also suffer from low bone mass (especially in males) and reduced grip strength and reduced force of fast twitch muscles. Bone as well as muscle tissues express GPR180. Accordingly, provided herein are methods for the treatment of at least steatosis, low bone mass/osteoporosis, and muscle weakness with agonists (and/or antagonists) of GPR180.

Collagen triple helix repeat containing 1 (Cthrc1) was identified in a screen for sequences induced in arteries upon balloon catheter injury (Pyagay et al., 2005 Circ Res, 96:261-268). The response to this injury induces smooth muscle cell proliferation with intimal thickening formation and constrictive remodeling with reduction in lumen size and fibrosis of the adventitia. In normal vessels, expression of Cthrc1 is not detectable, however, after injury, adventitial cells express Cthrc1 abundantly. Cthrc1-specific monoclonal antibodies suitable for immunohistochemistry were generated and validated extensively using tissues from Cthrc1 transgenic mice and Cthrc1 deficient mice (Stohn et al., 2012 PLoS One, 7:e47142, incorporated herein by reference). In the adult, expression of Cthrc1 was restricted to the hypothalamus, pituitary gland and bone (Stohn et al., 2012 PLoS One, 7:e47142), but no expression was identified in inner ear hair cells as reported by Yamamoto et al (Yamamoto et al., 2008 Dev Cell, 15:23-36) In addition, during processes associated with activation of fibroblasts and interstitial cells such as tissue repair and remodeling as well as cancer, Cthrc1 is induced in activated mesenchymal cells including cancer activated fibroblasts (Stohn et al., 2012 PLoS One, 7:e47142). The development of hepatic steatosis in Cthrc1 deficient mice in the absence of detectable Cthrc1 expression in the liver led to the identification that Cthrc1 is a hormone with pituitary and bone contributing to circulating levels.

Kimura reported decreased bone mass and decreased numbers of osteoblasts in Cthrc1 deficient mice on a mixed genetic background as well as stimulatory effects of Cthrc1 on osteoblast proliferation and differentiation (Kimura et al., 2008 PLoS ONE, 3:e3174). Kimura also reported reduced trabecular numbers, but similar trabecular thickness for mutants compared to wildtype mice. Transgenic mice overexpressing a 3×HA tagged form of Cthrc1 in bone had increased bone mass and compensated for bone loss associated with ovariectomy (Kimura et al., 2008 PLoS ONE, 3:e3174).

The results presented herein build upon previous findings of circulating Cthrc1. As a circulating factor, Cthrc1 might have effects on many organs. As such, tissue abnormalities in Cthrc1 null mice were investigated regardless of known expression of Cthrc1. Furthermore, as described in detail below, monoclonal antibodies were developed for the establishment of a sensitive sandwich ELISA and identification of Cthrc1 binding proteins.

### Collagen Triple Helix Repeat Containing-1 (Cthrc1) Protein

Collagen triple helix repeat containing-1 (CTHRC1) is a protein isolated from a cDNA library of injured arteries. CTHRC1 functions as an inhibitor of TGF-β signaling. CTHRC1 is susceptible to cleavage by proteases and purified CTHRC1 forms aggregates, making it difficult to perform cell binding studies and protein interaction studies. Expression analyses of CTHRC1 in tissues have been performed by in situ hybridization, immunohistochemistry and RT-PCR analysis. CTHRC1 has also been found in plasma. CTHRC1 plasma levels in healthy human volunteers ranged from 16-440 ng/ml.

### Steatosis

Steatosis (i.e., fatty change, fatty degeneration, or adipose degeneration) is the process describing the abnormal retention of lipids within a cell. Due to an impairment of the normal processes of synthesis and elimination of triglyceride fat, excess lipid accumulates in vesicles that displace the cytoplasm. Macrovesicular steatosis is a phrase used to describe the consition when the vesicles are large enough to distort the nucleus. Otherwise, the condition is known as microvesicular steatosis. While mild cases are not detrimental to the cell, large accumulations can disrupt cell constituents, and in severe cases the cell may even burst.

The risk factors associated with steatosis include diabetes mellitus, protein malnutrition, hypertension, cell toxins, obesity, anoxia, and sleep apnea. As the liver is the primary organ of lipid metabolism, it is most often associated with steatosis; however, it may occur in any organ, e.g., the kidneys, heart, and muscle.

The diagnosis of hepatic steatosis is made when fat in the liver exceeds 5-10% by weight (Reddy J K and Rao M S 2006, Am J Physiol Gastrointest Liver Physiol, 290(5): G852-8, incorporated herein by reference). Most individuals are asymptomatic and are usually discovered incidentally because of abnormal liver function tests or hepatomegaly noted in unrelated medical conditions. Elevated liver biochemistry is found in 50% of patients with simple steatosis. The serum alanine transaminase level usually is greater than the aspartate transaminase level in the nonalcoholic variant and the opposite in alcoholic fatty liver disease (AST:ALT more than 2:1). Imaging studies are often obtained during the evaluation process. Ultrasonography reveals a “bright” liver with increased echogenicity. Medical imaging can aid in diagnosis of fatty liver; fatty livers have lower density than spleens on computed tomography (CT), and fat appears bright in T1-weighted magnetic resonance images (MRIs). No medical imagery, however, is able to distinguish simple steatosis from advanced NASH. Histological diagnosis by liver biopsy is sought when assessment of severity is indicated.

Cardiac steatosis may be diagnosed with magnetic resonance spectroscopy and imaging (Nelson, et al., 2013 112(7): 1019-24, incorporated herein by reference).

### Osteoporosis

Osteoporosis is a progressive bone disease characterized by a decrease in bone mass and bone density, which often leads to an increased risk of fracture. In osteoporosis, the bone mineral density (BMD) is reduced, bone microarchitecture deteriorates, and the amount and variety of proteins in bone are altered. Osteoporosis is defined by the World Health Organization (WHO) as a bone mineral density of 2.5 standard deviations or more below the mean peak bone mass (average of young, healthy adults) as measured by dual-energy X-ray absorptiometry. The term “established osteoporosis” includes the presence of a fragility fracture. The disease may be classified as primary type 1, primary type 2, or secondary. Primary type 1 or postmenopausal osteoporosis is most commonly seen in women after menopause. Primary type 2 osteoporosis or senile osteoporosis occurs after age 75 and is seen in both females and males at a ratio of 2:1. Secondary osteoporosis may arise at any age and affects men and women equally. This form results from chronic predisposing medical problems or disease, or prolonged use of medications such as glucocorticoids (steroid- or glucocorticoid-induced osteoporosis). The diagnosis of osteoporosis can be made using conventional radiography and by measuring the bone mineral density (BMD; Guglielmi G and Scalzo G Diagnostic Imaging Europe, 26: 7-11, incorporated herein by rererence). The most popular method of measuring BMD is dual-energy x-ray absorptiometry. In addition to the detection of abnormal BMD, the diagnosis of osteoporosis requires investigations into potentially modifiable underlying causes; this may be done with blood tests.

### Muscle Weakness

Muscle weakness or myasthenia is a lack of muscle strength. The causes are many and can be divided into conditions that have either true or perceived muscle weakness based on its cause. True muscle weakness (i.e., neuromuscular weakness) is a primary symptom of a variety of skeletal muscle diseases, wherein the force exerted by the muscles is less than would be expected, e.g., in muscular dystrophy, inflammatory myopathy, and in neuromuscular junction disorders, such as myasthenia gravis. Muscle weakness can also be caused by low levels of potassium and other electrolytes within muscle cells. Perceived muscle weakness (i.e., non-neuromuscular weakness) is a condition wherein a person feels more effort than normal is required to exert a given amount of force despite normal actual muscle strength, e.g., in chronic fatigue syndrome.

The severity of muscle weakness can be classified into different “grades” based on the following criteria: Grade 0: No contraction or muscle movement; Grade 1: Trace of contraction, but no movement at the joint; Grade 2: Movement at the joint with gravity eliminated; Grade 3: Movement against gravity, but not against added resistance; Grade 4: Movement against external resistance, but less than normal; and Grade 5: Normal strength.

### Metabolic Syndrome

Metabolic syndrome is a disorder of energy utilization and storage, diagnosed by a co-occurrence of three out of five of the following medical conditions: abdominal (central) obesity, elevated blood pressure, elevated fasting plasma glucose, high serum triglycerides, and low high-density lipoprotein (HDL) levels. Metabolic syndrome increases the risk of developing cardiovascular disease and diabetes

The main sign of metabolic syndrome is central obesity (also known as visceral, male-pattern or apple-shaped adiposity), overweight with adipose tissue accumulation particularly around the waist and trunk. Other signs of metabolic syndrome include high blood pressure, decreased fasting serum HDL cholesterol, elevated fasting serum triglyceride level (VLDL triglyceride), impaired fasting glucose, insulin resistance, or prediabetes. Associated conditions include hyperuricemia, fatty liver (especially in concurrent obesity) progressing to nonalcoholic fatty liver disease, polycystic ovarian syndrome (in women), erectile dysfunction (in men), and acanthosis nigricans.

The International Diabetes Federation consensus worldwide definition of the metabolic syndrome (2006) is: Central obesity (defined as waist circumference^{#} with ethnicity-specific values) AND any two of the following:
Raised triglycerides: >150 mg/dL (1.7 mmol/L), or specific treatment for this lipid abnormality
Reduced HDL cholesterol: <40 mg/dL (1.03 mmol/L) in males, <50 mg/dL (1.29 mmol/L) in females, or specific treatment for this lipid abnormality
Raised blood pressure (BP): systolic BP >130 or diastolic BP >85 mm Hg, or treatment of previously diagnosed hypertension
Raised fasting plasma glucose (FPG): >100 mg/dL (5.6 mmol/L), or previously diagnosed type 2 diabetes
   If FPG is >5.6 mmol/L or 100 mg/dL, an oral glucose tolerance test is strongly recommended, but is not necessary to define presence of the syndrome. # If BMI is >30 kg/m², central obesity can be assumed and waist circumference does not need to be measured.

### Atherosclerosis

Atherosclerosis is a specific form of arteriosclerosis in which an artery wall thickens as a result of invasion and accumulation of white blood cells (WBCs). Atherosclerosis is a syndrome affecting arterial blood vessels due to a chronic inflammatory response of WBCs in the walls of arteries. This is promoted by low-density lipoproteins (LDL) without adequate removal of fats and cholesterol from the macrophages by functional high-density lipoproteins (HDL). It is commonly referred to as a “hardening” or furring of the arteries. It is caused by the formation of multiple atheromatous plaques within the arteries.

Areas of severe narrowing, stenosis, detectable by angiography, and to a lesser extent “stress testing” have long been the focus of human diagnostic techniques for cardiovascular disease, in general. However, these methods focus on detecting only severe narrowing, not the underlying atherosclerosis disease. Besides the traditional diagnostic methods such as angiography and stress-testing, other detection techniques have been developed in the past decades for earlier detection of atherosclerotic disease. Some of the detection approaches include anatomical detection and physiologic measurement. Examples of anatomical detection methods include (1) coronary calcium scoring by CT, (2) carotid IMT (intimal media thickness) measurement by ultrasound, and (3) intravascular ultrasound (IVUS). Examples of physiologic measurement methods include (1) lipoprotein subclass analysis, (2) HbAlc, (3) hs-CRP, and (4) homocysteine. Both anatomic and physiologic methods allow early detection before symptoms show up, disease staging and tracking of disease progression. In the recent years, ways of estimating the severity of atherosclerotic plaques is also made possible with the developments in nuclear imaging techniques such as PET and SPECT.

### Obesity

Obesity is a medical condition in which excess body fat has accumulated to the extent that it may have a negative effect on health, leading to reduced life expectancy and/or increased health problems. In Western countries, people are considered obese when their body mass index (BMI), a measurement obtained by dividing a person's weight by the square of the person's height, exceeds 30 kg/m², with the range 25-30 kg/m² defined as overweight, 30-35 kg/m² defined as class I obesity, 35-40 kg/m² defined as class II obesity, and 40 kg/m² or more defined as class III obesity.

### Diabetes

Diabetes mellitus (DM), commonly referred to as diabetes, is a group of metabolic diseases in which there are high blood sugar levels over a prolonged period. Symptoms of high blood sugar include frequent urination, increased thirst, and increased hunger. If left untreated, diabetes can cause many complications. Acute complications include diabetic ketoacidosis and nonketotic hyperosmolar coma. Serious long-term complications include cardiovascular disease, stroke, chronic kidney failure, foot ulcers, and damage to the eyes.

Diabetes is due to either the pancreas not producing enough insulin or the cells of the body not responding properly to the insulin produced. There are three main types of diabetes mellitus:
Type 1 DM results from the pancreas' failure to produce enough insulin. This form was previously referred to as “insulin-dependent diabetes mellitus” (IDDM) or “juvenile diabetes”.
Type 2 DM begins with insulin resistance, a condition in which cells fail to respond to insulin properly. As the disease progresses a lack of insulin may also develop. This form was previously referred to as “non insulin-dependent diabetes mellitus” (NIDDM) or “adult-onset diabetes”. The primary cause is excessive body weight and not enough exercise.
Gestational diabetes is the third main form and occurs when pregnant women without a previous history of diabetes develop a high blood sugar level.

### Polynucleotide Therapy

If desired, nucleic acid molecules that encode a GPR180 agonist (or antagonist) are delivered to a subject having or at risk of developing steatosis, low bone mass/osteoporosis, or muscle weakness. The nucleic acid molecules are delivered to the cells of a subject in a form in which they can be taken up so that therapeutically effective levels of the therapeutic polypeptide or fragment thereof can be produced.

A variety of expression systems exists for the production of therapeutic polypeptides. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (e.g., pET-28) (Novagen, Inc., Madison, Wis). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Alternatively, recombinant polypeptides of the invention are expressed in Pichia pastoris, a methylotrophic yeast. Pichia is capable of metabolizing methanol as the sole carbon source. The first step in the metabolism of methanol is the oxidation of methanol to formaldehyde by the enzyme, alcohol oxidase. Expression of this enzyme, which is coded for by the AOX1 gene is induced by methanol. The AOX1 promoter can be used for inducible polypeptide expression or the GAP promoter for constitutive expression of a gene of interest.

Once the recombinant polypeptide of the invention is expressed, it is isolated, for example, using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra). Alternatively, the polypeptide is isolated using a sequence tag, such as a hexahistidine tag, that binds to nickel column.

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a stem cell recruiting factor, variant, or a fragment thereof, can be cloned into a retroviral vector and expression can be driven from its endogenous promoter, from the retroviral long terminal repeat, or from a promoter specific for a tissue or cell of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the introduction of a therapeutic to a cell of a subject (e.g., a cell or tissue). For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88:4626-4630 (1991)), adenosine deaminase, phosphoglycerol kinase (PGK), pyruvate kinase, phosphoglycerol mutase, the actin promoter (Lai et al., Proc. Natl. Acad. Sci. USA 86: 10006-10010 (1989)), and other constitutive promoters known to those of skill in the art. In addition, many viral promoters function constitutively in eukaryotic cells. These include: the early and late promoters of SV40; the long terminal repeats (LTR) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified stem cell and/or in a cell of the tissue having a deficiency in cell number. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the subject.

In addition to at least one promoter and at least one heterologous nucleic acid encoding the therapeutic agent, the expression vector preferably includes a selection gene, for example, a neomycin resistance gene, for facilitating selection of stem cells that have been transfected or transduced with the expression vector.

If desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant stem cell recruiting factor, variant, or fragment thereof, either directly to the site of a potential or actual disease-affected tissue or systemically (for example, by any conventional recombinant protein administration technique). The dosage of the administered protein depends on a number of factors, including the size and health of the individual subject. For any particular subject, the specific dosage regimes should be adjusted over time according to the individual need and the professional judgment of the person administering or supervising the administration of the compositions.

### Pharmaceutical Compositions and Administration

The present invention comprises pharmaceutical preparations comprising a human GPR180 agonist (or antagonist) polypeptide together with a pharmaceutically acceptable carrier. Such compositions are useful for the treatment or prevention of fatty liver disease, low bone mass, and muscle weakness, or for the prevention or treatment of any one or more of the risk factors associated with these conditions. Polypeptides of the invention may be administered as part of a pharmaceutical composition. The compositions should be sterile and contain a therapeutically effective amount of the polypeptides in a unit of weight or volume suitable for administration to a subject.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups also can be derived from inorganic bases such as, for example, sodium, potassium, ammonium, calcium or ferric hydroxides, and such organic bases as isopropylamine, trimethyl amine, 2-ethylamino ethanol, histidine, procaine and the like. Particularly preferred are the salts of TFA and HCl.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions also can contain liquid phases in addition to and to the exclusion of water. Exemplary of such additional liquid phases are glycerin, vegetable oils such as cottonseed oil, and water-oil emulsions.

A therapeutic composition contains an inflammation inhibiting amount or a fibrosis inhibiting amount of an Cthrc1 polypeptide of the present invention, typically formulated to contain an amount of at least 0.1 weight percent of Cthrc1 polypeptide per weight of total therapeutic composition. A weight percent is a ratio by weight of inhibitor to total composition. Thus, for example, 0.1 weight percent is 0.1 grams of inhibitor per 100 grams of total composition.

These compositions can be stored in unit or multi-dose containers, for example, sealed ampoules or vials, as an aqueous solution or as a lyophilized formulation for reconstitution. As an example of a lyophilized formulation, 10 mL vials are filled with 5 mL of sterile-filtered 1% (w/v) aqueous Cthrc1 polypeptide solution, and the resulting mixture can then be lyophilized. The infusion solution can be prepared by reconstituting the lyophilized material using sterile Water-for-Injection (WFI).

The compositions can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

The dosage ranges for the administration of the Cthrc1 polypeptide vary. In general, amounts are large enough to produce the desired effect in which disease symptoms of a metabolic syndrome are ameliorated. The dosage should not be so large as to cause adverse side effects. Generally, the dosage will vary with the age, condition, sex and extent of the disease in the patient and can be determined by one of skill in the art. The dosage also can be adjusted by the individual physician in the event of any complication.

A therapeutically effective amount is an amount sufficient to produce a measurable inhibition of symptoms of a condition (e.g., an increase in bone mass or a decrease in muscle weakness). Such symptoms are measured in conjunction with assessment of related clinical parameters.

A therapeutically effective amount of a polypeptide of this invention in the form of a polypeptide, or fragment thereof, is typically an amount of polypeptide such that when administered in a physiologically tolerable composition is sufficient to achieve a plasma concentration of from about 0.1 microgram (ug) per milliliter (mL) to about 200 ug/mL, or from about 1 ug/mL to about 150 ug/mL. In one embodiment, the plasma concentration in molarity is from about 2 micromolar (uM) to about 5 millimolar (mM) or from 100 uM to 1 mM Cthrc1 polypeptide. In other embodiments, the doses of polypeptide ranges from about 500 mg/Kg to about 1.0 g/kg (e.g., 500, 600, 700, 750, 800, 900, 1000 mg/kg).

The agents of the invention can be administered parenterally by injection or by gradual infusion over time. In other embodiments, agents are administered intravenously, intraperitoneally, intramuscularly, subcutaneously, intracavity, transdermally, topically, intraocularly, orally, intranasally, and can be delivered by peristaltic means. In one embodiment, a therapeutic compositions containing an agent of this invention are administered in a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered and timing depends on the patient to be treated, capacity of the patient's system to utilize the active ingredient, and degree of therapeutic effect desired. Precise amounts of active ingredient required to be administered depend on the judgement of the practitioner and are peculiar to each individual. However, suitable dosage ranges for systemic application are disclosed herein and depend on the route of administration. Suitable regimes for administration also are variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

GPR180 agonists (or antagonists) can be administered as a peptide, either modified or unmodified, for example to modify the pharmacokinetic and/or pharmacodynamic properties of the peptide. A GPR180 peptide can be administered as a full-length peptide, or as a peptide not including the signal sequence (corresponding to amino acids 1 to 30 of the sequence).

The amount and frequency of administration of GPR180 would depend on a number of factors including, but not limited to, the condition to be treated.

### Therapy

As demonstrated herein, GPR180 agonists (or antagonists) are useful for the treatment or prevention of steatosis, low bone mass (i.e., osteoporosis), and muscle weakness. Subjects suffering, suspected of suffering, or prone to these conditions can be tested and monitored for expression levels of CTHRC1. Determining CTHRC1 levels can be performed at a single time point, or CTHRC1 levels can be monitored over time, as are many diagnostic markers, and substantial changes in CTHRC1 levels can be an indication that further testing for a metabolic disorder should be performed. Testing can be done using any assay specific for CTHRC1, for example any immunoassay, preferably an assay that is amenable to high throughput and/or automated screening methods. Antibodies to various portions of CTHRC1 can be generated using routine methods. Methods of epitope selection, antigen preparation, and antibody production are well known to those of skill in the art.

Therapy employing a GPR180 agonist polypeptide or a polynucleotide encoding a GPR180 agonist polypeptide is also provided by the invention. Therapy may be provided wherever therapy for these conditions is performed: at home, the doctor's office, a clinic, a hospital's outpatient department, or a hospital. Treatment generally begins at a hospital so that the doctor can observe the therapy's effects closely and make any adjustments that are needed. The duration of the therapy depends on the kind of disease being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

A GPR180 agonist (or antagonist) polypeptide or a polynucleotide encoding a GPR180 agonist polypeptide may be administered within a pharmaceutically-acceptable diluent, carrier, or excipient, in unit dosage form. Conventional pharmaceutical practice may be employed to provide suitable formulations or compositions to administer the compounds to patients suffering from a disease that is associated with a metabolic syndrome. Administration may begin before the patient is symptomatic. Any appropriate route of administration may be employed, for example, administration may be topical, parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a disease or condition. “Therapeutically effective amount” is intended to include an amount of a compound useful in the present invention or an amount of the combination of compounds claimed, e.g., to treat or prevent the disease or disorder, or to treat the symptoms of the disease or disorder, in a host. The combination of compounds is preferably a synergistic combination. Synergy, as described for example by Chou and Talalay, Adv. Enzyme Regul. 22:27-55 (1984), occurs when the effect of the compounds when administered in combination is greater than the additive effect of the compounds when administered alone as a single agent. In general, a synergistic effect is advantageously demonstrated at suboptimal concentrations of the compounds. Synergy can be in terms of lower cytotoxicity, increased activity, or some other beneficial effect of the combination compared with the individual components. The preferred dosage of a GPR180 agonist polypeptide or a polynucleotide encoding a GPR180 agonist polypeptide is likely to depend on such variables as the type and extent of the disorder, the overall health status of the particular patient, the formulation of the compound excipients, and its route of administration. If desired, treatment with an agent of the invention may be combined with therapies for the treatment of a metabolic syndrome.

For any of the methods of application described above, an agent of the invention of the invention is desirably administered intravenously or is applied to the tissue affected by metabolic syndrome (e.g., by injection).

### Kits

The invention provides kits for the treatment or prevention of a metabolic syndrome. In one embodiment, the kit includes a therapeutic or prophylactic composition containing an effective amount of an agent described herein. In some embodiments, the kit comprises a sterile container that contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired an agent of the invention is provided together with instructions for administering the agent to a subject having or at risk of developing a metabolic syndrome. The instructions will generally include information about the use of the composition for the treatment or prevention of a metabolic syndrome. In other embodiments, the instructions include at least one of the following: description of the therapeutic agent; dosage schedule and administration for treatment or prevention of a metabolic syndrome or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

It was previously reported that CTHRC1 in the adult is expressed in the hypothalamus, pituitary gland, and bone and circulating levels of Cthrc1 are detectable in human plasma. Furthermore, Cthrc1 null mice develop severe hepatic steatosis. Described herein, is the investigation of bone and muscle abnormalities in Cthrc1 null mutant mice, the determination of the range of Cthrc1 levels in human plasma, and the identification of a Cthrc1 receptor.

As described in detail below, Cthrc1 null mice on the C57Bl/6 background have severely reduced bone mass, that is more prominent in males than females. Furthermore, lack of Cthrc1 is associated with reduced grip strength and reduced strength of fast twitch muscles, whereas muscle fiber type analyses revealed no abnormalities. A monoclonal antibody-based capture ELISA detected significantly higher levels of Cthrc1 in healthy men than women, and dramatically elevated levels in some patients suffering from a variety of conditions. Using recombinant Cthrc1 and tissue homogenates from Cthrc1 deficient mice, CTHRC1 ligand-receptor complexes were purified with highly specific monoclonal antibodies. G protein-coupled receptor 180 (GPR180) was identified by mass-spectrometry as a receptor for Cthrc1. Similar to the discovery of Cthrc1 in injured rat arteries, GPR180 (intimal thickness-related receptor, ITR), was originally identified as a gene overexpressed in injured rabbit aortae. Unlike Cthrc1, GPR180 is widely expressed.

As described in more detail below, effective therapies for lowering cholesterol levels and improving lipid profiles are established and are being widely prescribed to prevent and treat atherosclerosis. Despite these therapeutic successes, atherosclerosis has remained the most common cause of death. Therefore, there is still a significant need to identify and evaluate therapeutic targets for the treatment and prevention of cardiovascular disease.

Apart from cholesterol lowering statins, Pparγ agonists (thiazolidinedione, TZDs) are beneficial in slowing development and progression of atherosclerosis in animal models and humans with type II diabetes (T2DM). This was unexpected because oxidized LDL particles as endogenous Pparγ ligands were thought to promote a vicious cycle characterized by Pparγ activation leading to increased CD36 expression with increased lipid uptake and hence accelerated atheroslcerosis. It was later suggested that the beneficial effects of TZDs on atherosclerosis may at least in part be mediated by increasing HDL-cholesterol. In addition, a more recent study reported that the TZDs inhibit atherosclerosis progression independent of their effects on hyperglycemia, insulin resistance and dyslipidemia, indicating direct actions of the TZDs on the vessel wall. Combined, these studies show that prior to the invention described herein, the mechanism of atheroprotective effects of Pparγ is uncertain.

Described herein is the role of Cthrc1 in inhibition of Pparγ expression. As described in more detail below, the absence of Cthrc1 in mice is associated with significantly increased expression of Pparγ and its target genes. Furthermore, Cthrc1 null mice on an atherosclerosis prone ApoE null background have lower total cholesterol and increased HDL-cholesterol levels accompanied by a 50% reduction in atherosclerosis. As described in detail below, using ELISAs, elevated circulating levels of Cthrc1 was detected in diseases associated with accelerated atherosclerosis such as diabetes. In addition, the absence of Cthrc1 is associated with low bone mass and increased body fat, both known side effects of TZD treatments. Cthrc1 is also identified as a circulating factor that binds to its candidate receptor Gpr180. This ligand-receptor system strongly influences lipid metabolism, body composition, and atherogenesis.

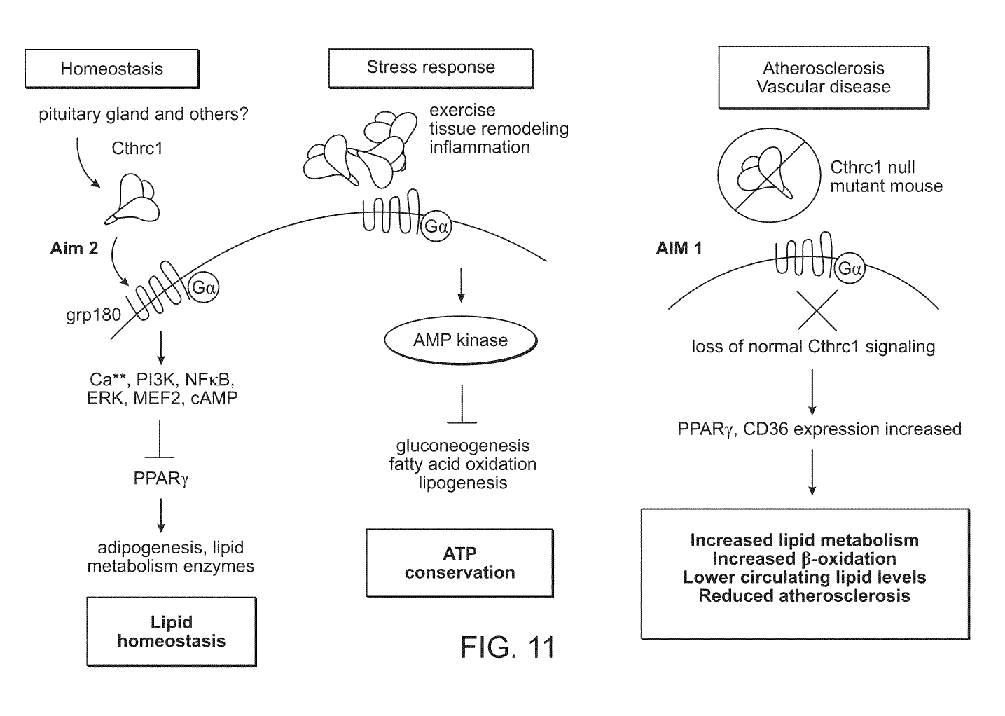

Described herein is the role of the Cthrc1-Gpr180 ligand-receptor system in health and disease. Described herein is the elucidation of the nature of Cthrc1-Gpr180 signaling and its functional influence on atherogenesis, the major mechanisms involved, and the translational potential they offer for prevention or management of atherosclerosis. As described in detail below, Cthrc1 functions as a factor contributing to regulation of cellular lipid metabolism through its interaction with its cognate receptor GPR180, which in turn affects Pparγ and its downstream targets as well as AMPK activation. A schematic of the function of Cthrc1 is shown in FIG. 11.

Specifically, described herein is the underlying mechanism for reduced atherosclerosis in the absence of Cthrc1 by determining i) whether Cthrc1 regulates expression of Pparγ and downstream targets in smooth muscle cells, monocyte/macrophages and adipocytes, ii) whether Cthrc1 regulates fatty acid uptake and lipid storage in vitro and in vivo, and iii) whether Cthrc1 regulates lipolysis and fatty acid oxidation. The latter includes comprehensive metabolic monitoring.

Also described herein is the characterization of signaling of Cthrc1 through GPR180 with respect to receptor activation, involved G-proteins, downstream signaling pathways and identification of downstream targets.

This invention is further illustrated by the following examples, which should not be construed as limiting. All documents mentioned herein are incorporated herein by reference.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1

### GPR180 is a Receptor for CTHRC1

### Animals

Cthrc1 null mice with deletion of exons 2, 3, and 4 were previously described (Stohn et al., 2012 PLoS One, 7:e47142) and maintained on pure C57BL/6J (Jackson Laboratories) and 12956/SvEv (Taconic) background by backcrossing for more than 11 generations. Mice were fed a standard rodent diet (Harlan, 2018 Teklad Global 18% Protein Rodent Diet) and water ad libitum, and housed with dry cellulose bedding (Harlan, 7070 Diamond) under a 14 hour daylight-10 hour night cycle.

Forelimb grip strength was measured using a digital force-gauge meter (PCE-FG50, PCE GmbH, 59872 Meschede, Germany) with a custom grip bar attachment. Mice were held by the base of their tail above the grip bar and allowed to grab it. Mice were then gently pulled upwards until they released their grip. The maximum force pulled was recorded for each mouse.

For myography studies, twelve week old Cthrc1 null mice and wildtype mice on the C57Bl/6J background were anesthetized with ketamine/xylazine and exsanguinated. The soleus and extensor digitorum longus (EDL) muscles were removed and placed in a bath of Ringer's solution gas-equilibrated with 95% 0₂/5% CO₂. The muscles were subjected to isolated muscle mechanical measurements using a previously described apparatus (Aurora Scientific, Aurora, ON, Canada; Barton E R, 2006 J Appl Physiol (1985), 100:1778-1784). After determining optimum length using single supramaximal twitch stimulation, maximum isometric tetanus was measured.

RNA isolation and RT-PCR

Total RNA from tissues was isolated using TRI REAGENT (Sigma-Aldrich). RNA integrity was confirmed by 1% agarose gel electrophoresis; 1.0 μg of the total RNA were then reverse transcribed using the ImProm-II reverse transcriptase (Promega) according to the manufacturer's protocols. Semi-quantitative PCR was performed using GPR180 primers (GGAGCAGTTTGAAGACACCAGTCAC (SEQ ID NO: 1) and TGGCACAAGAACCACAGGATGC (SEQ ID NO: 2)) and GAPDH primers (ATCACTGCCACCCAGAAGACTG (SEQ ID NO: 3) and ATCCACGACGGACACATTGG (SEQ ID NO: 4).

### Microcomputed Tomography (μCT)

Microarchitecture of the trabecular bone and midshaft cortical bone of the femur was analyzed by μCT (resolution 10 μm, VivaCT-40; Scanco Medical AG, Bassersdorf, Switzerland). Bones were scanned at energy level of 55 kVp and intensity of 145 μA. Trabecular bone volume fraction and microarchitecture were evaluated in the secondary spongiosa, starting approximately at 0.6 mm proximal to the distal femoral growth plate and extending proximally 1.5 mm. Approximately 230 consecutive slices were made at a 10.5 μm interval at the distal end of the growth plate and extending in a proximal direction, and 180 contiguous slices were selected for analysis. Measurements included bone volume/total volume, trabecular number (Tb.N.), trabecular thickness (Tb.Th.), and trabecular spacing. Scans for the cortical region were measured at the midpoint of each femur, with an isotropic pixel size of 21 μm and slice thickness of 21 μm, and used to calculate the average BA, total cross-sectional area, BA/total cross-sectional area, and Ct.Th. For midshaft analysis, the cortical shell was contoured by user-defined threshold and iterated across the 50 slices. All scans were analyzed using manufacturer software (version 4.05; Scanco Medical AG).

### Human Plasma Samples

Samples were obtained from healthy adult male and female volunteers after obtaining informed consent. All plasma was processed and frozen within 4 hours of the blood draw and stored at −70° C. until analyzed.

### Monoclonal Antibody and ELISA Development

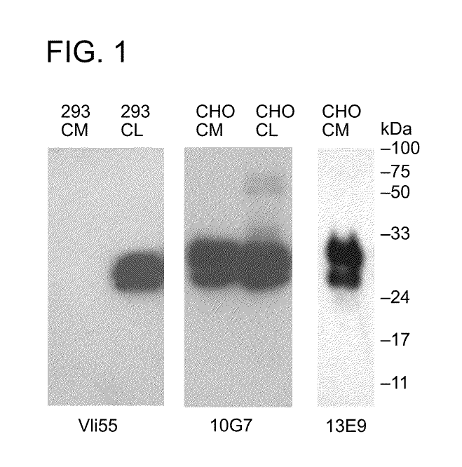

Mice were immunized with synthetic peptides corresponding to the N terminus of human and murine Cthrc1 (SENPKVKQKAQLRQRE (SEQ ID NO: 5) and SENPKVKQKALIRQRE (SEQ ID NO: 6)) coupled to keyhole limpet hemocyanin (KLH) as well as recombinant rat Cthrc1 with C terminal 8×His expressed in E. coli. Using the services of Maine Biotechnology Services (Portland, Me.), hybridomas were generated. Hybridoma supernatants were screened by indirect ELISA against human and rat Cthrc1 expressed in CHO-K1 cells. In addition, the supernatants were also screened by indirect ELISA against the N terminal peptides conjugated to bovine serum albumin (BSA) as well as by immunoblot analysis of rat and human Cthrc1. All clones referenced here detected a single band of approximately 30 kDa on immunoblots of lysates and conditioned media of Cthrc1 transfected CHO-K1 cells run under reducing conditions (FIG. 1).

Monoclonal antibodies 10G07, 13D11, and 19C07 are specific for the N terminus of human Cthrc1 and do not react with rat or murine Cthrc1. Clone 13E09 recognizes an epitope located within the N terminal half of the molecule of both human and rodent Cthrc1.

Clone 13E09 is suitable as a capturing antibody in a sandwich ELISA and biotinylated 10G07 (OneQuant Sulfo-NHS-LC Biotin, G-Biosciences) was used as detection antibodies for human Cthrc1, respectively. It is important to note that none of the monoclonal antibodies generated recognized C terminally modified Cthrc1 by ELISA, even though their epitopes mapped to the N terminal half of the molecule. This included Cthrc1 with C terminal myc tag, myc/6×His tag, 8×His tag, or Cthrc1 with deletion of the C terminal lysine (K) residue.

Cthrc1 standards were prepared from purified Cthrc1 with a 7×His tag fused to the N terminus and expressed in CHO-K1 cells. For analysis of plasma samples for Cthrc1 levels, ELISA plates (MAXISORP, Nunc) were coated at 4° C. overnight with 100 μl of anti-Cthrc1 13E09 at 1.8 μg/ml in carbonate buffer (pH9.6). Plates were washed four times between all incubations with 400 μl per well of PBS-T containing 0.1% BSA. Wells were blocked with 400 μl of PBS-T containing 0.1% BSA for 1 hour at room temperature (r.t.) on a plate shaker. Samples of 100 μl of undiluted plasma were incubated for 2 hours at r.t., followed by incubation with biotinylated anti-Cthrc1 10G07 used at 0.5 μg/ml for 45 min. Streptavidin-HRP (Vector Laboratories) was used at 1:1000 dilution followed by the colorimetric reaction with TMB substrate (TMB PLUS LIQUID, Amresco) as directed. Optical densities were read at 450 nm with plate reader (Apollo LB913, Berthold Technologies). For detection of low levels of Cthrc1 a biotinyl tyramide amplification system was used as directed (ELAST ELISA Amplification system, Perkin-Elmer). The detection limit of the assay is approximately 1 pg/ml.

### Immunohistochemistry, Immunoblotting and Cell Culture

A rabbit monoclonal anti-Cthrc1 clone Vli-55 was used for Western blotting and immunohistochemistry on paraffin-embedded, paraformaldehyde-fixed tissues as described (Stohn et al., 2012 PLoS One, 7:e47142). A full-length open reading frame expression clone for murine GPR180 was obtained (clone ID 6336270, ThermoFisher) and a C terminal myc epitope tag was fused to it in frame for detection with anti-myc antibodies (rabbit monoclonal Vli-1 (Cuttler et al., 2011 Genesis) and mouse monoclonal 9E10). HEK293-T and CHO-K1 cells were grown as described and transfected with the expression vector for GPR180 using Fugene6 HD (Roche) (LeClair et al., 2007 Circ Res, 100:826-833). For binding studies, GPR180 and control transfected cells were incubated with untagged hCthrc1 expressed in CHO-K1 cells at a concentration of 125 ng/ml. Cells were fixed with formalin after the indicated incubation periods and stained for immunofluorescence confocal microsopy. Incubation of cells with anti-myc Vli-1 (20 ng/ml, overnight at 4° C.) was followed by incubation with Alexa Fluor 488 conjugated anti-rabbit IgG (1:500 dilution for 2 hours, Jackson ImmunoResearch). To prevent dissociation of the bound anti-rabbit IgG, cells were again fixed for 5 min with formalin prior to incubation with biotinylated anti-Cthrc1 Vli-55 for detection of bound Cthrc1. Bound anti-Cthrc1 was visualized with streptavidin conjugated Alexa Fluor 546 (Invitrogen).

### Cthrc1 Binding Studies In Vivo and In Vitro

One μg of purified 7×His-hCthrc1 was pre-incubated with HRP-conjugated (EZ-Link Plus Activated Peroxidase, Pierce) anti-Cthrc1 10G07 for 10 min prior to intra-arterial infusion via the common carotid artery into anesthetized Cthrc1 null mice. Controls received anti-Cthrc1 10G07-HRP without Cthrc1. Five minutes after the infusion, the cardiovascular system was perfused extensively with Lactated Ringer's solution (20 ml) to remove blood products from all organs. Various organs were harvested and processed for frozen sections. Sections were fixed briefly with formalin before detecting bound Cthrc1-antibody complexes using diaminobenzidine (DAB) as color substrate as described (Stohn et al., 2012 PLoS One, 7:e47142).

Previous immunolocalization studies had indicated that there is widespread expression of Cthrc1 in the remodeling mesenchyme after birth. This includes the vascular and musculo-skeletal system. It was reasoned that potential Cthrc1 receptors may be expressed at the same time that Cthrc1 is expressed in remodeling tissues. Thus, the liver and the gastrointestinal tract was removed from two day old Cthrc1 null pups. The remaining tissue was homogenized under liquid nitrogen. Crude tissue homogenates were incubated with 1 μg of purified hCthrc1 for 30 min on ice in the presence of protease inhibitors, followed by incubation with a cocktail of anti-human Cthrc1 monoclonal antibodies for 4 hours on ice. Protein A Sepharose was used to isolate hCthrc1-antibody complexes. Proteins were identified by liquid nanoscale liquid chromatography-mass spectrometry (LC-MS) as described previously (Young et al., 2012 Blood). Protein A Sepharose bound proteins were digested with sequencing grade trypsin (Sigma) and the resulting tryptic peptides were isolated using reverse phase C18 spin columns (PepClean C18 column, Pierce). Extracted peptides were analyzed by LC-MS using via quadrupole-time-of-flight mass spectrometry and linear ion trap (QSTAR, 4000QTRAP, respectively, ABSciex) mass spectrometers. For mass spectrometry, peptides were separated using a linear 0-60% water/acetonitrile gradient (0.1% formic acid) on a U_{—}3000 RSLC nanoscale capillary liquid chromatograph (ThermoFisher-Dionex) fitted with a Acclaim PepMap reversed-phase capillary column (3 μm, C18, 100 Angstrom pore size, 75 μm ID×15 cm, 15 μm tip; LC Packings) and an inline PepMap 100 precolumn (C18, 300 μm ID×5 mm; LC Packings), which served as a loading column.

Peak list generation, peptide mass fingerprint peak picking, and relative quantification and protein identification were performed with the ProteinPilot™ software (ABSciex). Default parameters were used for all analyses. Protein database searches were conducted using the UniProtKB/Swiss-Prot protein knowledgebase release 2013_{—}10. MS/MS spectra were searched against a database of mouse protein sequences. Threshold score for accepting individual MS/MS spectra was 95% confidence, based on confirmation by western blot analysis. Trypsin autolysis peaks and keratin tryptic peptides were known contaminants that were excluded from database searching.

Mass-spectrometry identified only 19 proteins indicating that the approach was very focused. Among the proteins identified were Cthrc1, six different immunoglobulin chains and the G-protein coupled receptor GPR180. Using RT-PCR, many cell lines were found to express endogenous levels of GPR180 including HEK293-T cells and Pac1 smooth muscle cells; however, no Cthrc1 binding was observed in CHO-K1 cells, which were used in subsequent binding and co-localization studies after transfecting the cells with the GPR180myc plasmid (GeneJuice, EMD-Millipore).

### Statistical Analyses

Data are expressed as means±standard deviation. Student's t-test was used for all calculations. P<0.05 was considered significant.

### Cthrc1 Plasma Levels in Human Subjects

Based on previous report of circulating Cthrc1 in human subjects (Stohn et al., 2012 PLoS One, 7:e47142), a monoclonal antibody-based capture ELISA able to detect low pg/ml concentrations of Cthrc1 in plasma was developed. Because Cthrc1 is susceptible to cleavage by proteases such as plasmin (Leclair et al., 2008 Arterioscler Thromb Vasc Biol, 28:1332-1338), plasma samples were utilized in the experiments. Plasma samples were obtained from healthy human subjects.

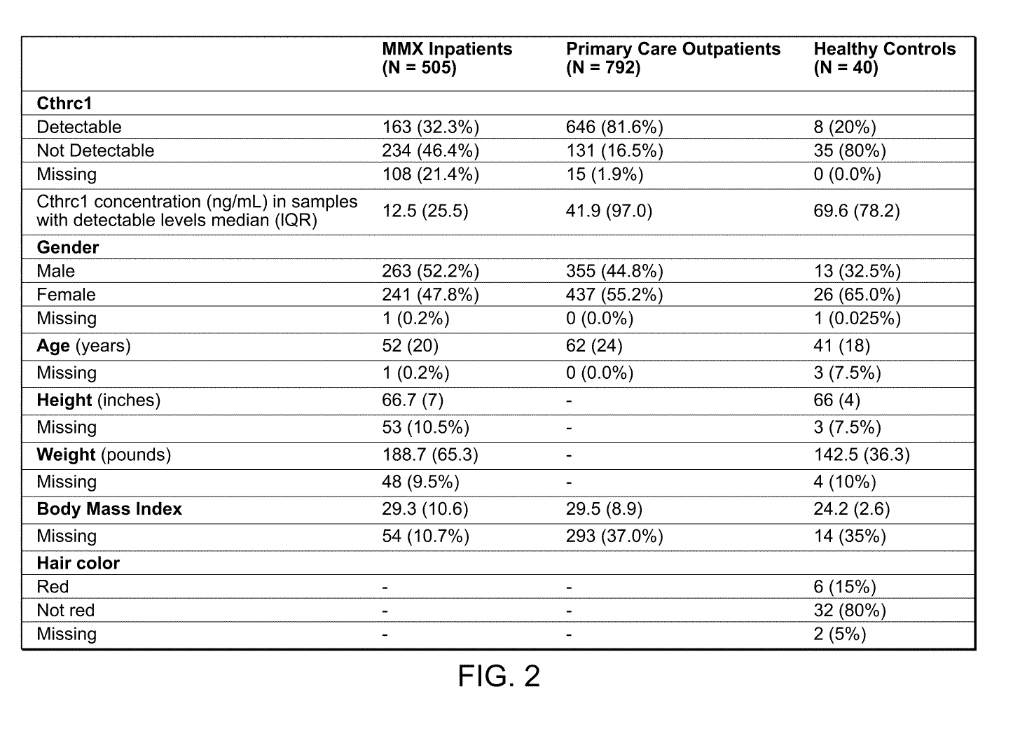

FIG. 2 shows the basic clinical and demographic characteristics of a patient population analyzed for levels of Cthrc1. The patient samples came from two clinical sites: Maine Medical Center Inpatient Unit (N=505), and an outpatient primary care facility in southern Maine (N=792), as well as a set of healthy volunteer controls (N=40). The Cthrc1 levels varied across different sites. Higher Cthrc1 levels were seen at the outpatient location (median concentration of 41.9 ng/mL as opposed to 12.5 ng/mL at MMC) potentially affected by the time to processing and type of anticoagulant used. The normal controls had a high average concentration, but this was accounted for by the disproportionate overrepresentation of red haired individuals. Cthrc1 plasma levels for healthy individuals excluding this sub-population were below the detection limit of the assay. For this reason, comparisons in Cthrc1 levels are made within and not between patient data sets.

### Bone Mass in Male Versus Female Cthrc1 Null Mice

Kimura (Kimura et al., 2008 PLoS ONE, 3:e3174) reported decreased bone mass and decreased numbers of osteoblasts in Cthrc1 mutant mice harboring a deletion of exon 2. These mice were on a predominantly C57Bl/6 genetic background. Bones were evaluated separately for male and female Cthrc1 null mice on a pure C57Bl/6J background.

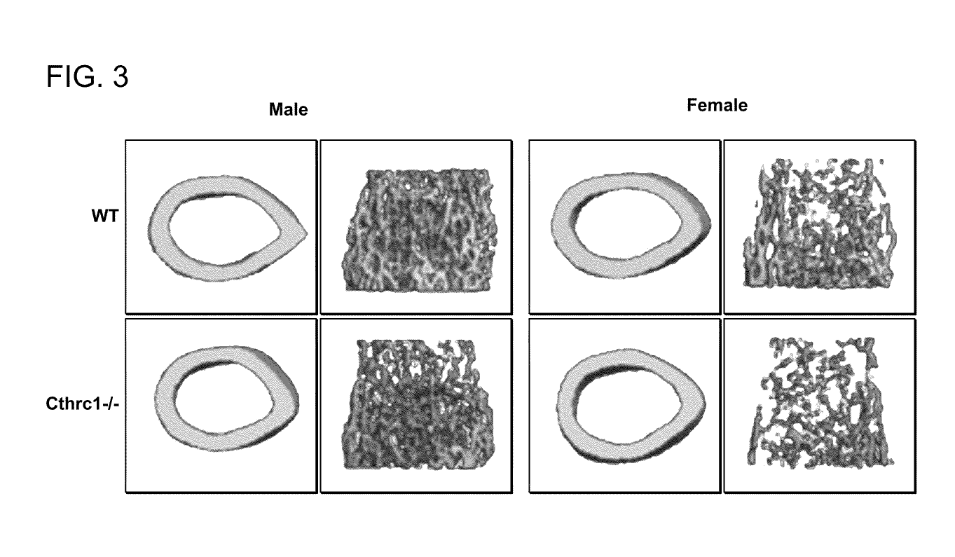

A series of bone μCT images of femurs from 8 week old C57Bl/6J wildtype and Cthrc1 null mice on the same background are shown in FIG. 3. Note the reduction in trabecular bone.

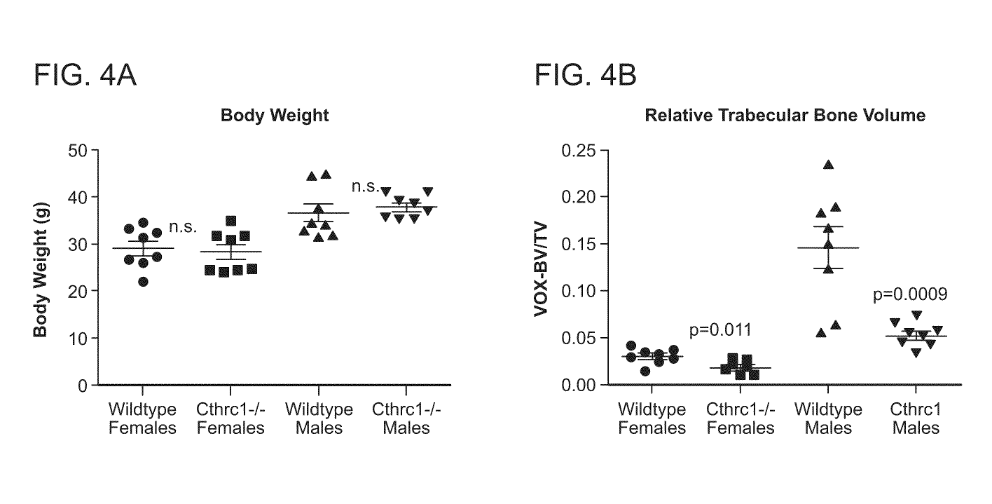

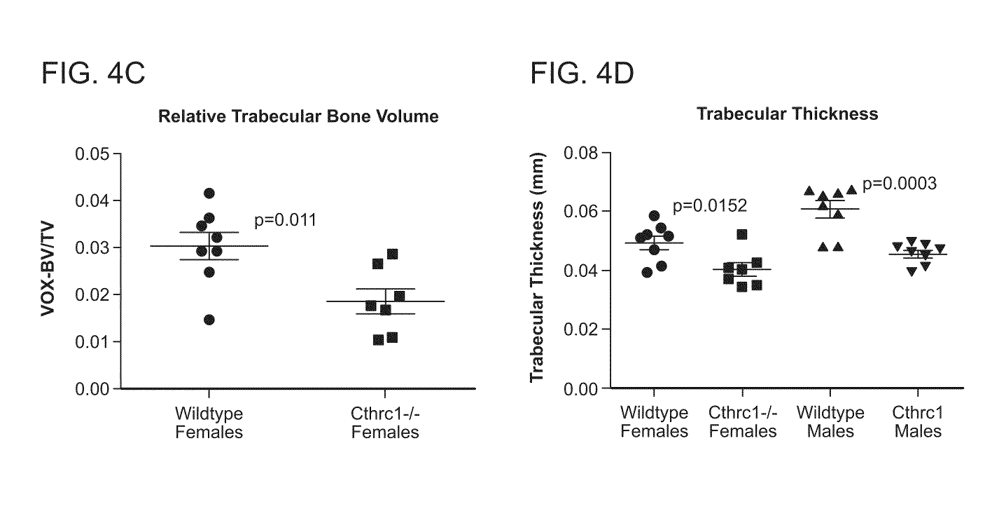

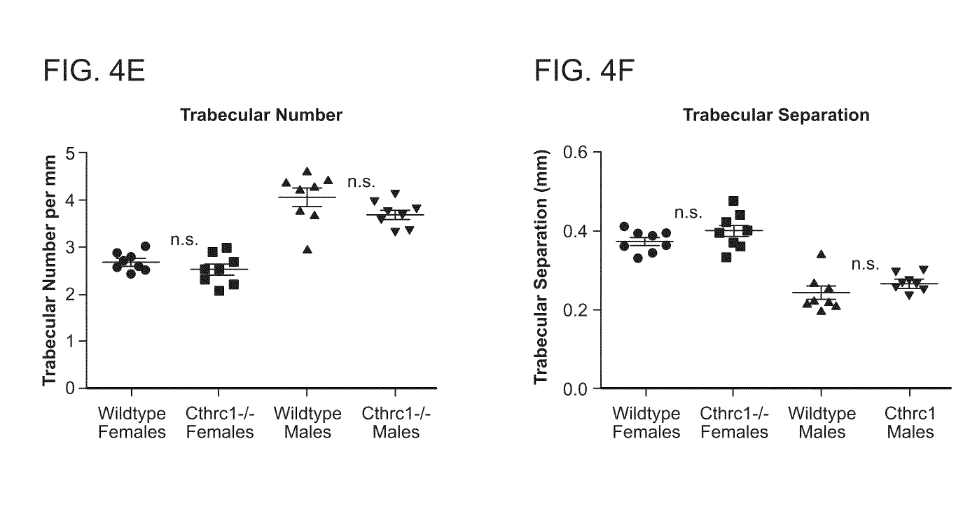

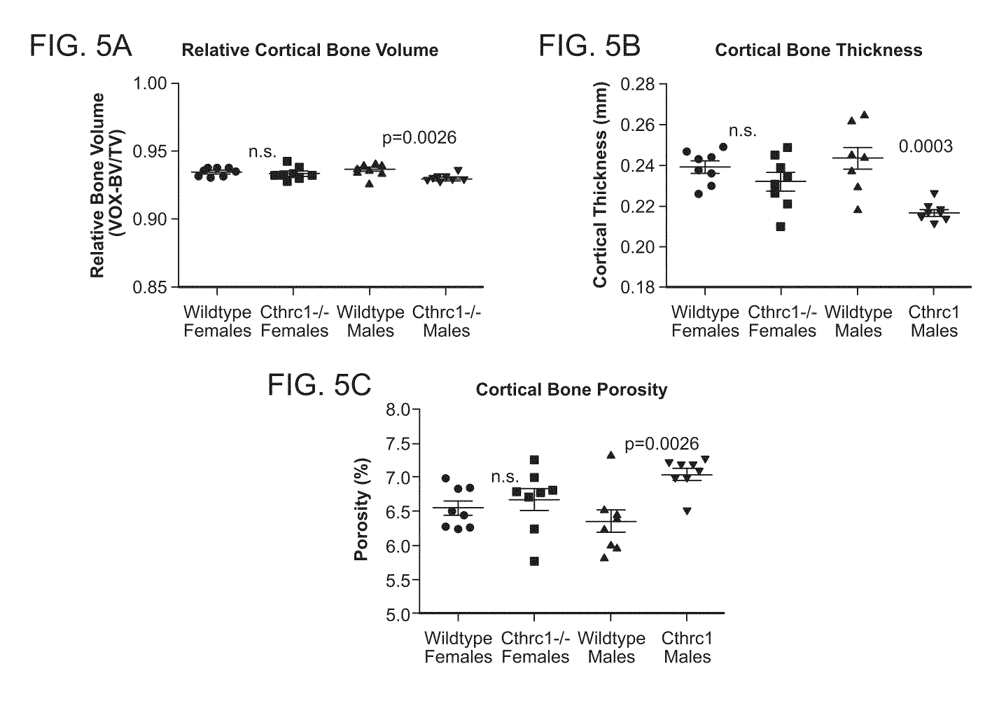

Femurs from 25 week old Cthrc1 null mice and corresponding wildtype mice on the C57Bl/6J background were analyzed. This was performed by μCT separately for males and females. FIG. 4A is a dot plot showing body weight. FIG. 4B is a dot plot showing relative trabecular bone volume. FIG. 4C is a dot plot showing relative trabecular bone volume. FIG. 4D is a dot plot showing trabecular thickness. FIG. 4E is a dot plot showing trabecular number. FIG. 4F is a dot plot showing trabecular separation. Trabecular bone volume and trabecular thickness were significantly reduced in Cthrc1 null mice. FIG. 5A shows relative cortical bone volume. FIG. 5B shows cortical bone thickness. FIG. 5C shows cortical bone porosity. Cortical bone thickness was severely reduced in male Cthrc1 null mice, but not females. Correspondingly, cortical bone porosity was significantly increased only in males.

### Reduced Grip Strength and Muscle Force in Cthrc1 Null Mice

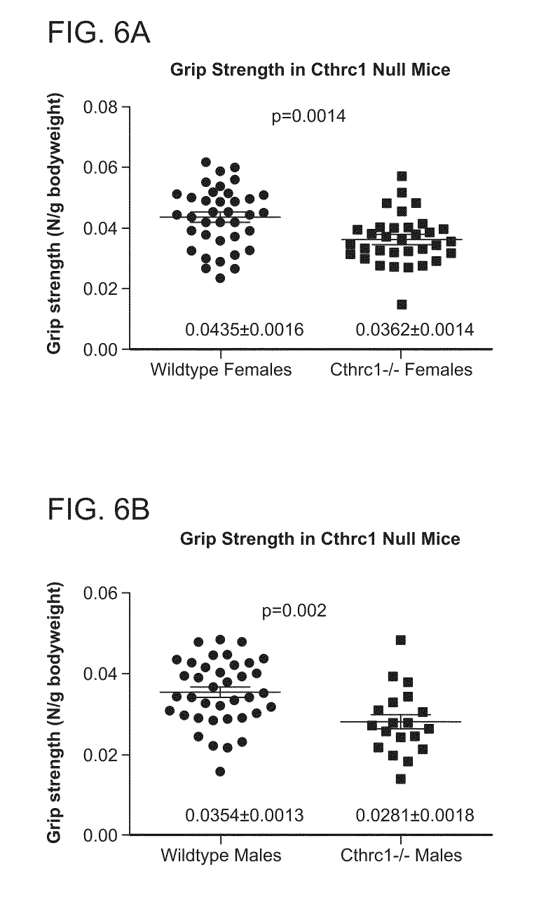

Grip strength measurements were performed in Cthrc1 null mice and corresponding wildtype mice as described above (FIG. 6A and FIG. 6B).

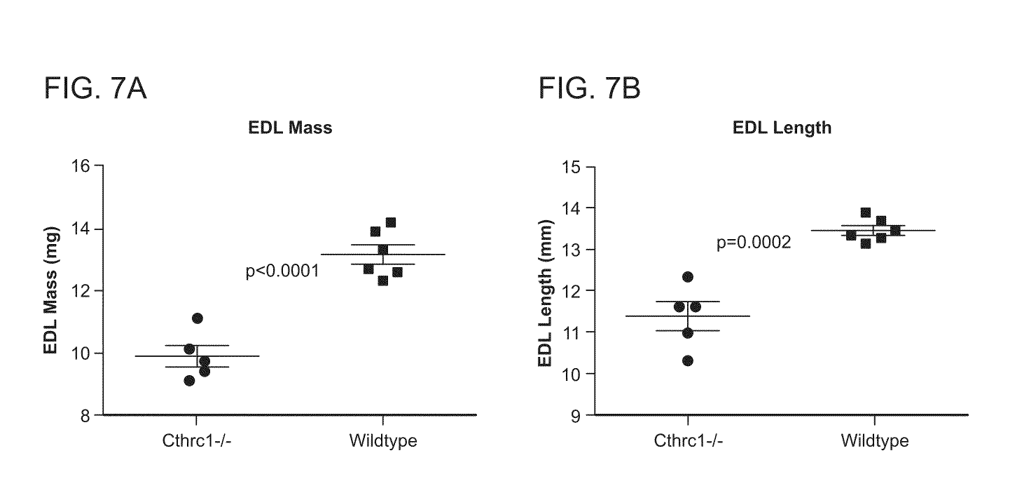

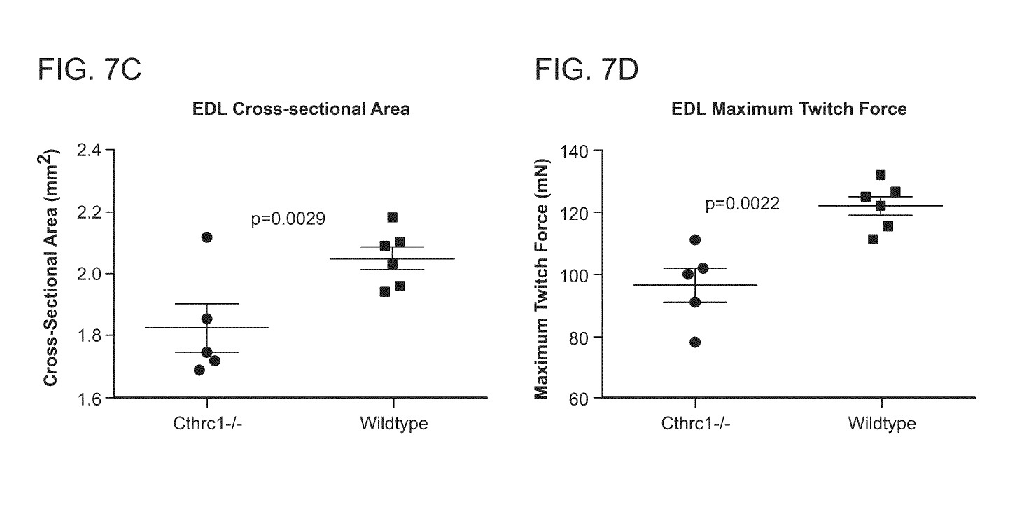

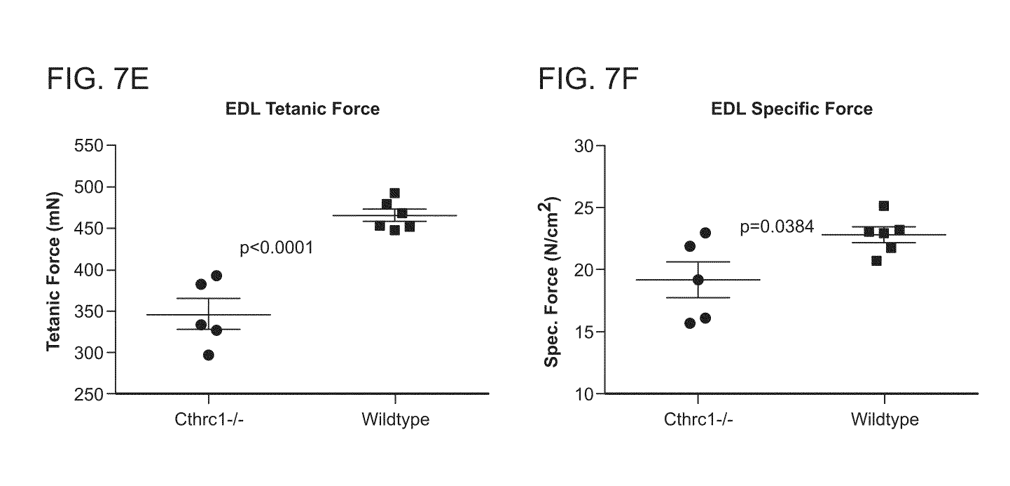

The extensor digitorum longus muscle (EDL), a fatigueable fast twitch muscle, was examined by ex vivo myography. The mass and length of the EDL was significantly reduced along with reduced cross-sectional fiber area in Cthrc1 null mice (FIG. 7A-FIG. 7F).

Maximum twitch force, as well as tetanic force, were significantly reduced in EDL muscles from Cthrc1 null mice. The difference in muscle force could not be explained by the decrease in muscle mass because the specific EDL force with normalization to muscle cross-sectional area was also reduced (FIG. 7A-FIG. 7F). This indicates that muscle mass of fast twitch muscles is regulated by Cthrc1. Furthermore, absence of Cthrc1 is associated with reduced muscle force even when normalized to muscle cross-sectional area.

### GPR180 is a Receptor for Cthrc1

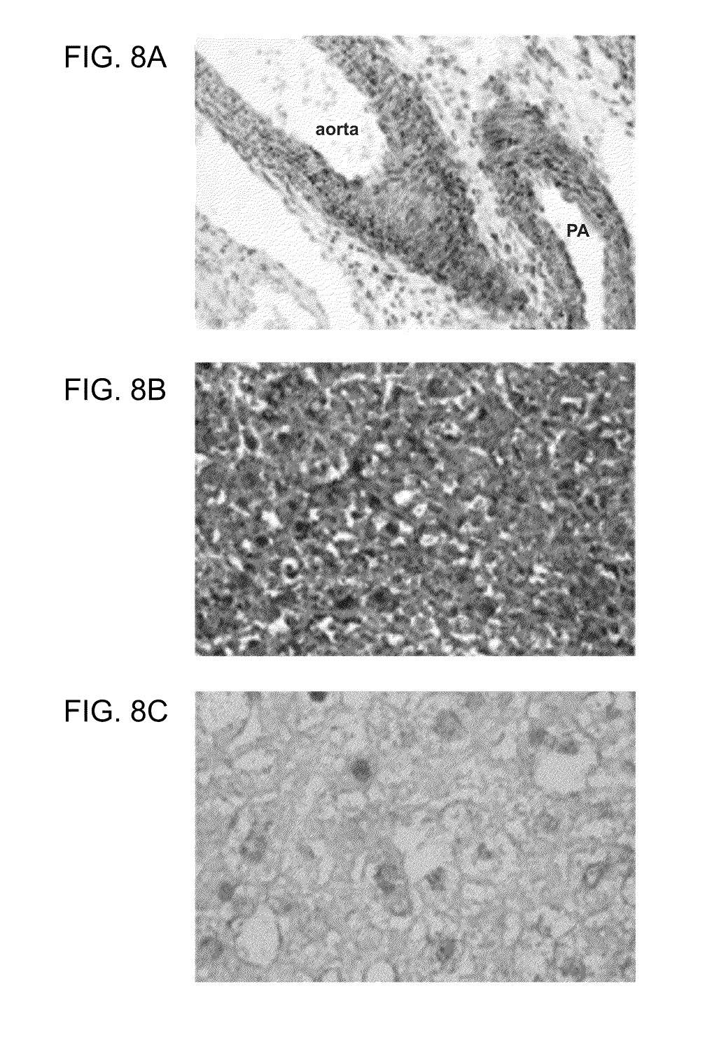

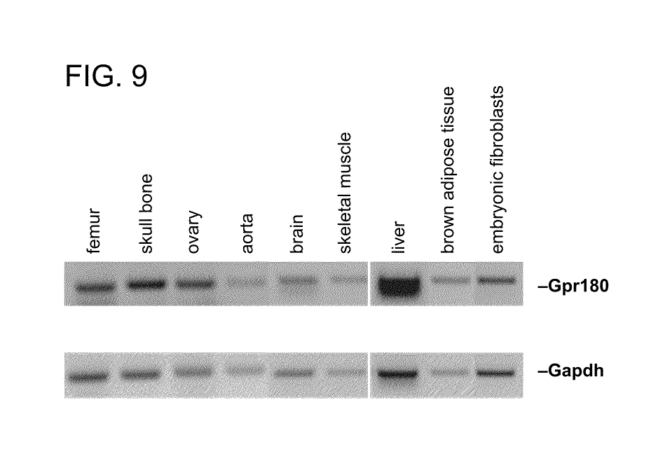

Purified hCthrc1 was pre-incubated with HRP-conjugated anti-Cthrc1 10G07 prior to intra-arterial infusion via the common carotid artery into anesthetized Cthrc1 null mice. Controls received anti-Cthrc1 10G07-HRP without Cthrc1. The bound Cthrc1 was visualized in tissue sections via the HRP activity. Binding of Cthrc1 was prominent in the liver (FIG. 8A-FIG. 8C). Previous immunolocalization studies had indicated that there is widespread expression of Cthrc1 in the remodeling mesenchyme after birth. This includes the vascular and musculo-skeletal system (FIG. 8A). It was reasoned that potential Cthrc1 receptors are expressed at the same time that Cthrc1 is expressed in remodeling tissues. Crude tissue homogenates from two day old Cthrc1 null pups were incubated with hCthrc1 followed by incubation with a cocktail of anti-human Cthrc1 monoclonal antibodies. Cthrc1 bound to potential receptors was isolated and putative binding proteins identified by nanoscale liquid chromatography-mass spectrometry (LC-MS) as described previously (Young et al., 2012 Blood). GPR180 was among the proteins identified by mass-spectrometry. Using RT-PCR, it was identified that GPR180 was widely expressed (FIG. 9). Originally, GPR180 was identified as a G protein-coupled receptor expressed in rabbit aortae in response to balloon injury, and the authors named it intimal thickness-related receptor (ITR) (Tsukada et al., 2003 Circulation, 107:313-319). Prior to the invention described herein, a ligand that binds to GPR180 had not yet been identified. Cthrc1 was identified in a screen for genes induced in arteries in response to balloon injury.

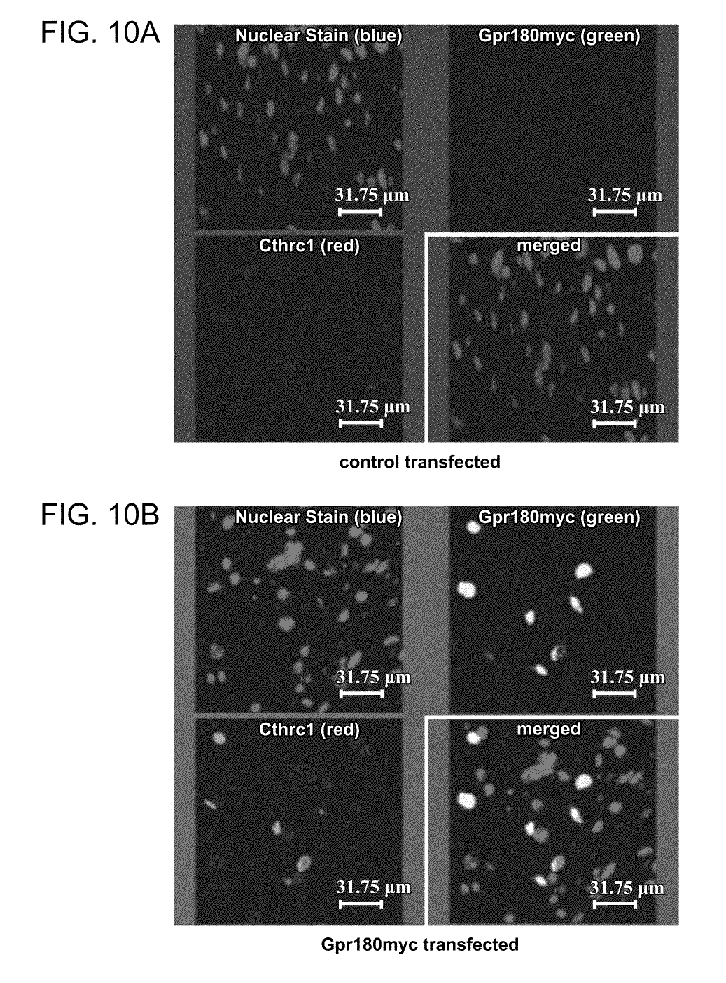

Next, experiments were performed to confirm that GPR180 binds CTHRC1. This was performed with co-localization studies performed in vitro. Initial studies revealed that Cthrc1 bound to a variety of cells without prior transfection of the cells with the myc-tagged GPR180 expression vector including HEK293-T cells and Pac1 smooth muscle cells; however, no CTHRC1 binding was observed in CHO-K1 cells. Endogenous GPR180 expression was confirmed for HEK293-T cells and Pac1 smooth muscle cells by RT-PCR as well as mouse embryonic fibroblasts (FIG. 9). Control transfected CHO-K1 cells were grown to confluence and incubated with hCthrc1 (125 ng/ml) for 10 minutes on ice. Unbound Cthrc1 was removed by washing the cells with ice cold PBS followed by with formalin. Bound Cthrc1 was detected by immunofluorescence confocal microscopy. Bound Cthrc1 could not be detected in control transfected cells (FIG. 10A). However, binding of Cthrc1 was detectable in cells previously transfected with the GPR180 receptor (FIG. 10B). Binding of Cthrc1 co-localized perfectly with cells that expressed the myc-tagged GPR180, identified by anti-myc immunocytochemistry (FIG. 10B).

In summary, as described in detail above, using mass-spectrometry and co-localization studies, it was identified that GPR180 is a receptor for Cthrc1. This receptor is expressed in many organs and very prominently in the liver. It has previously reported that Cthrc1 null mice develop severe hepatic steatosis (Stohn et al., 2012 PLoS One, 7:e47142). As described herein, Cthrc1 null mice suffer from low bone mass and reduced strength of fast twitch muscles. Bone as well as muscle tissues express GPR180. Furthermore, using anti-Cthrc1 ELISA reactive monoclonal antibodies, it was demonstrated that Cthrc1 is a circulating factor. As such, conditions associated with elevated Cthrc1 plasma levels could be diagnosed utilizing Cthrc1 as a biomarker. With the identification of GPR180 as a/the Cthrc1 receptor, GPR180 is a suitable drug target for conditions associated with CTHRC1. For example, as described herein, GPR180 specific agonists are used to treat fatty liver disease and muscle weakness.

### Example 2

### The Role of Cthrc1 in Artherosclerosis

Effective therapies for lowering cholesterol levels and improving lipid profiles are established and are being widely prescribed to prevent and treat atherosclerosis. Despite these therapeutic successes, atherosclerosis has remained the most common cause of death¹⁻³. Therefore, prior to the invention described herein, there was a significant need to identify and evaluate therapeutic targets for the treatment and prevention of cardiovascular disease.

Described herein is the identification of a factor and its candidate cognate receptor, which strongly influence lipid metabolism, body composition, and atherogenesis. Described in detail below is the elucidation of the nature of their functional influence on atherogenesis, the major mechanisms involved, and the translational potential they offer for prevention or management of atherosclerosis.

Collagen triple helix repeat containing-1 (Cthrc1) is induced in the activated adventitial fibroblast after vascular injury⁴. With the successful development of antibody reagents, it was determined that Cthrc1 is a circulating factor involved in regulation lipid metabolism. As described in detail below, Cthrc1 null mice on the apolipoprotein E (ApoE) null background develop approximately 50% less atherosclerosis on a high fat diet. Furthermore, a candidate receptor for Cthrc1, the G protein-coupled receptor GPR180, which is expressed in many tissues including the vasculature and macrophages was identified. Thus, a ligand-receptor system that plays a role in atherosclerosis and vascular disease has been determined. As described in detail below, unique monoclonal antibody-based sandwich ELISAs were developed. Elevated levels of Cthrc1 were identified in diseases associated with accelerated atherosclerosis such as diabetes. As described in detail below, genetic loss-of-function and inducible gain-of-function mouse models were generated to study the consequences of altered Cthrc1 levels for atherosclerosis and metabolic disease. In addition to the genetic models, all necessary reagents and assays were developed to study the functions of the Cthrc1-GPR180 ligand-receptor system in health and disease. G protein-coupled receptors are highly suitable for development of small molecule agonists and inhibitors. Described herein is the utilization of GPR-180 in therapy.

### Identification of Cthrc1

Cthrc1 was discovered in a screen for genes induced in response to vascular injury. Following balloon catheter angioplasty, Cthrc1 was prominently induced in cells of the adventitia with no expression detectable in normal arteries.⁴ Cthrc1 is only found in chordates and is highly conserved with approximately 98% identical amino acid sequence between mouse and human. Cthrc1 is also a secreted molecule with a signal peptide and a short Gly-X-Y (n=12) repeat, which is the only domain that has similarity with known proteins. Short Gly-X-Y repeats are also found in other proteins, such as the hormone adiponectin and other members of the CTRP (Clq and TNF receptor related proteins) family. Cthrc1 has a similar molecular mass and forms oligomers like adiponectin, but it lacks the defining Clq domain of CTRP family members, making it a unique protein.

A schematic showing the function of Cthrc1 is shown in FIG. 11. In healthy adults, low baseline levels of circulating Cthrc1 are maintained, influencing lipid metabolism via inhibition of Pparγ. Cthrc1 signals via its widely-expressed cognate receptor GPR180 to inhibit adipogenesis and lipogenesis. During inflammatory and stress conditions including tissue remodeling, pregnancy and diabetes, Cthrc1 levels are elevated, so as to conserve ATP levels via adenosine monophosphate-activated protein kinase (AMPK) activation. Cthrc1 deficiency causes increased FA oxidation, lowering of blood lipids with reduction in atherosclerosis.

### Cthrc1 is a Circulating Factor Elevated in Inflammatory Conditions and Diabetes

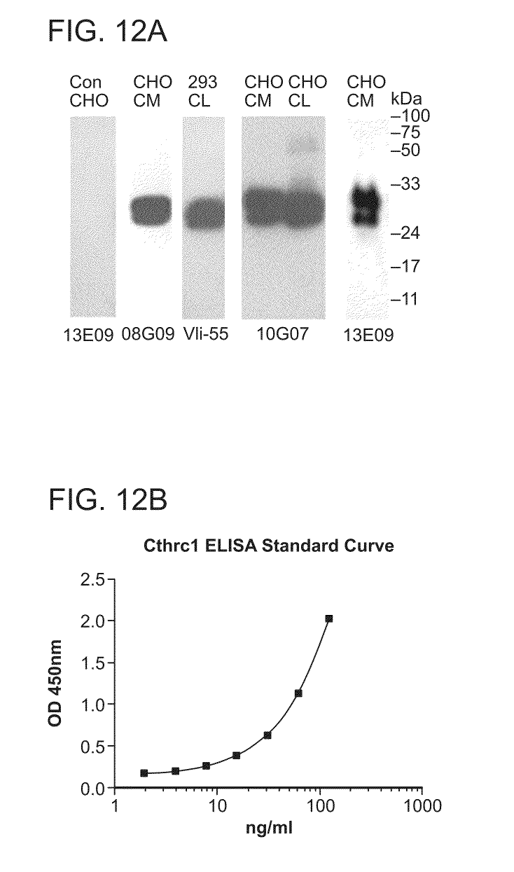

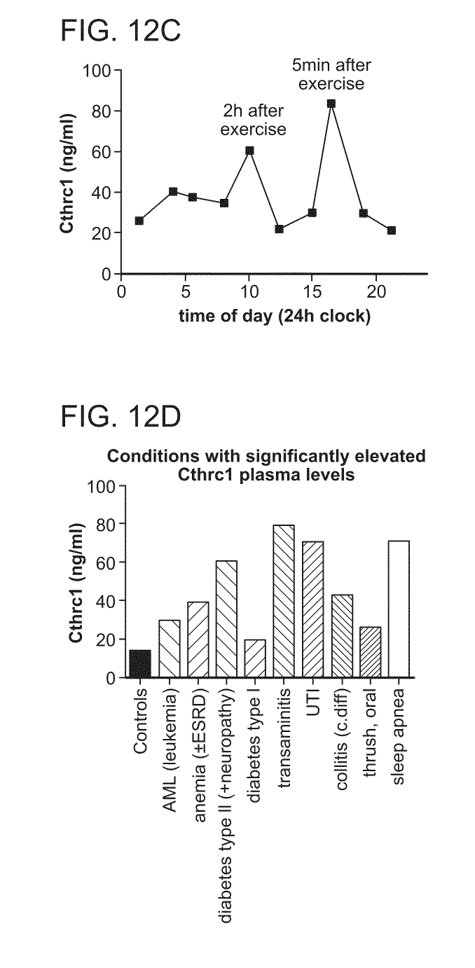

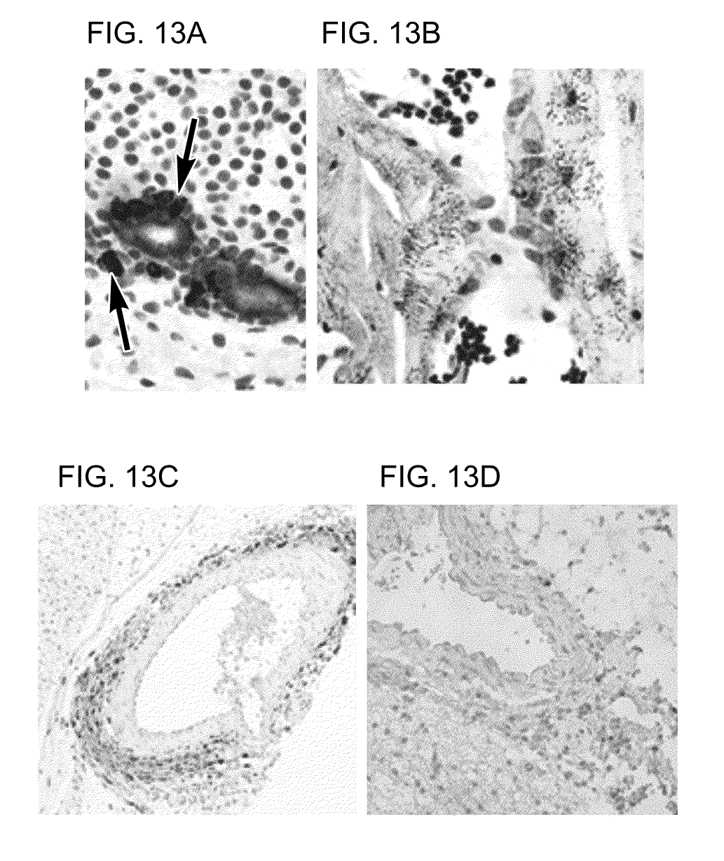

As described herein, mouse and rabbit monoclonal Cthrc1 specific antibodies that allowed detection by immunohistochemistry, Western blotting and sandwich ELISA were generated (FIG. 12A-FIG. 12D and FIG. 13A-FIG. 13D). In healthy human subjects, Cthrc1 circulates at ng/ml concentrations (FIG. 12C). Screening of 1300 random patient plasma samples found significantly elevated Cthrc1 levels in inflammatory conditions including type 2 diabetes (FIG. 12D). Cthrc1 is not detectable in the vasculature of healthy adult mice; however, injury and remodeling stimuli induce its expression in the fibroblast-like cell of the adventitia or any remodeling connective tissue (FIG. 13C). The anterior pituitary and bone tissues likely contribute to circulating Cthrc1 levels in healthy individuals (FIG. 13A and FIG. 13B)⁵. With the exception of parts of the brain, these tissues are the only tissues that constitutively expressed Cthrc1 in the adult.

### Atherosclerosis is Reduced in the Absence of Cthrc1

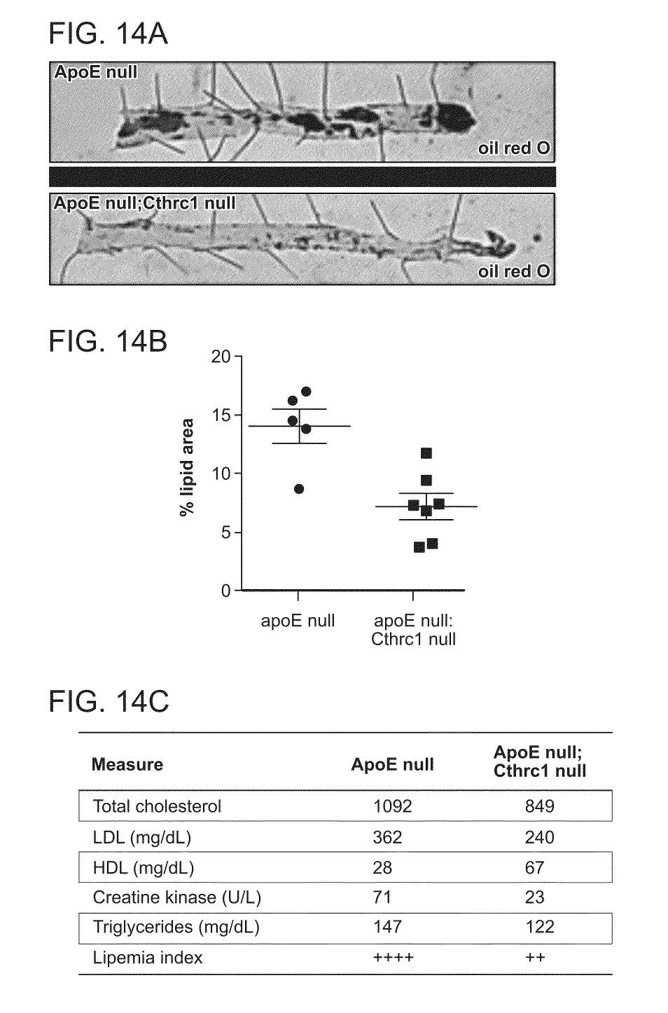

Cthrc1 null mice on the C57Bl6/J background were crossed with apolipoprotein E (ApoE) null mice (Jackson Laboratories) and double homozygous null mice were fed a Western diet for 16 weeks starting at 3 weeks of age (FIG. 14A-FIG. 14C). The aortic surface area stained with oil red O was reduced by approximately 50% in the double mutant mice (FIG. 14A and FIG. 14B). Spontaneous atherosclerosis on normal chow diet was similarly reduced. Serum cholesterol and triglycerides were measured in ApoE null and ApoE/Cthrc1 double mutant mice after 16 weeks on the WD. Double mutant mice had a less atherogenic lipid profile (FIG. 14C).

### Cthrc1 Regulates Body Composition

To determine if Cthrc1 null mice have weaker muscles, systematic analyses of grip strength was performed using a standard grip strength force meter. In addition, whole body composition was analyzed by NMR (Bruker Minispec) and comprehensive metabolic and activity monitoring was performed using a caging system (Promethion, Sable Systems Int.). Precise measurements of oxygen consumed and carbon dioxide produced allowed calculation of energy expenditure and respiratory quotients (RQ). A lower RQ indicates more fat oxidation. The system also provides data on food and water intake, sleep and activity in all three dimensions. A more detailed discussion of the data is provided below. Some of the parameters that were different between wildtype and Cthrc1 null mice are summarized in Table 1. The extensor digitorum longus muscle (EDL), a muscle with predominantly fast twitch fibers, was also excised and its mass and maximal tetanic twitch force determined. The data show that Cthrc1 null mice have similar body weight, but decreased lean mass (muscle) and increased body fat, suggesting regulation of body composition by Cthrc1. The reduced grip strength and EDL mass as well as reduced tetanic force generated are likely the consequence of an overall reduction in muscle mass.

TABLE 1

| | | | p = |
|---|---|---|---|
| | Wildtype Mice | Cthrc1 Null Mice | Value |
| Body Weight (g) | 28.85 ± 0.61 | 27.13 ± 1.07 | 0.196 |
| Lean Body Mass (g) | 22.58 ± 0.68 | 19.70 ± 0.25 | 0.018 |
| Body Fat (%) | 15.95 ± 0.80 | 22.93 ± 2.31 | 0.028 |
| Food Consumption | 0.1406 ± 0.0049 | 0.1304 ± 0.0096 | 0.355 |
| (g/24 h)/Lean Body | | | |
| Mass (g) | | | |
| Running Wheel Speed | 0.263 ± 0.014 | 0.201 ± 0.015 | 0.033 |
| (average, m/s) | | | |
| Grip strength (N/g, | 0.03542 ± 0.001305 | 0.02806 ± 0.001868 | 0.002 |
| 16 wk old males) | (n = 38) | (n = 19) | |
| Extensor digitorum | 0.50 ± 0.02 | 0.42 ± 0.05 | 0.007 |
| longus muscle (EDL) | (n = 6) | (n = 6) | |
| mass (mg/g body | | | |
| weight) | | | |

Table 1 shows the results of comprehensive metabolic and activity monitoring performed on 11 week old wildtype and Cthrc1 null mice (n=4 per group). Grip strength and EDL mass were determined in 16 week old mice.

### Absence of Cthrc1 Promotes a Lipogenic Gene Expression Program

As described previously, Cthrc1 null mice develop fatty livers that are readily detectable after the age of three months. Guided by the additional finding of increased body fat and reduced muscle mass, tissue and disease-oriented expression analyses were performed using the fatty liver-, muscle-, and glucose metabolism PCR based screening arrays (SA-Biosciences, Qiagen). This quantitative PCR-based approach was used to examine expression levels of genes relevant to lipid and glucose metabolism as well as liver and muscle disease. RNA was isolated from two day old Cthrc1 null and wildtype pups (pooled RNA samples from 3 pups per genotype with skin and intestines removed). At this time point, pronounced Cthrc1 expression was observed in the remodeling cardiovascular system and other mesenchymal tissues, suggesting that there is also increased Cthrc1 signaling at that time. A liver phenotype with pathologic histology is not apparent until later in adulthood and grip strength is reduced in Cthrc1 null mice after 10 weeks of age. Examining gene expression at this early time point provides better insight into any cause-effect relationships. Some genes with different expression levels by PCR array were verified by separate qRT-PCR with different primer sets. Expression levels of several genes involved in lipid metabolism (Pparγ, fatty acid synthase, CD36) were consistently higher in Cthrc1 null pups (Table 2). Many of the same genes are also overexpressed in mice with transgenic overexpression of Pparγ or TZD treatment^{6, 7}, e.g. CD36, Fasn, Cpt1a, adiponectin, Abca1, Acox1, and Srebf1 (Srebp1).

TABLE 2

| | Fold | | | Fold |
|---|---|---|---|---|
| Genes associated | Change | | Genes associated | Change |
| with lipid | (Cthrc1- | | with lipid | (Cthrc1- |
| metabolism | KO/WT) | | metabolism | KO/WT) |
| Adipoq | 1.63 | | Acsl5 (lipid | 1.38 |
| (adiponectin) | | | synthesis long | |
| | | | chain fatty acid | |
| | | | and degradation) | |
| Pparγ | 1.62 | (0.029) | Acsm3 (lipid | 1.35 |
| | | | synthesis medium | |
| | | | chain fatty acid) | |
| Ppargc1a (Pgc1α, | 1.22 | | Abca1 (reverse | 1.34 |
| fatty acid = | | | cholesterol | |
| metabolism, | | | transport to | |
| thermogenesis) | | | apoA-I) | |
| CD36 (fatty | 1.74 | (p = 0.001) | Acox1 (peroxisomal | 1.19 |
| acid | | | fatty acid | |
| translocase) | | | oxidation) | |
| Slc27a5 (fatty | 1.76 | (p = 0.05) | Pdk4 (fatty acid | 1.46 |
| acid | | | and glucose | |
| transporter) | | | metabolism) | |
| Fasn (fatty | 1.29 | (p = 0.049) | Adipor1/2 | 1.82/1.37 |
| acid | | | (adiponectin | |
| synthase) | | | receptor) | |

| | | | |
|---|---|---|---|
| Srebf1/Srebf2 | 1.2/1.12 | Cpt1a | 1.54 |
| (SREBP1, lipid | | (mitochondrial | |
| homeostasis, | | fatty acid uptake, | |
| cholesterol | | beta-oxidation) | |
| synthesis, | | | |
| LDL rec. | | | |
| expression) | | | |

Table 2 shows that analysis of gene expression in 2 day old wildtype and Cthrc1 null pups shows increased expression of genes involved in lipid metabolism and inflammation in Cthrc1 null mice. qRT-PCR analysis was performed on pooled cDNA from 3 whole body pups per genotype (intestines removed). Validation of expression was performed for some genes by independent qRT-PCR and the p values are shown.

### Identification of a Cthrc1 Receptor Candidate Identified as Gpr180

With evidence of Cthrc1 as a circulating factor with potential hormone-like functions, it was next determined if the effects are mediated via a Cthrc1-receptor interaction. Assuming that relatively abundant mesenchymal expression of Cthrc1 after birth coincides with increased Cthrc1 signaling, two day old Cthrc1 null pups were homogenized and the homogenate was incubated with purified Cthrc1. Proteins interacting with Cthrc1 were then isolated by incubation with a cocktail of monoclonal antibodies specific for native Cthrc1. The protein A purified antibody bound complexes were processed for mass-spectrometry after trypsin digestion. Two complementary protein identification platforms were used: Tandem_{—} quadrupole time-of-flight (qTOF, QSTAR, ABSciex) and linear ion trap (LIT, 4000QTRAP, ABSciex) mass spectrometers, both housed in the MMCRI vascular biology-COBRE Nucleic Acid, Protein Analysis and Cell Imaging core facility. Using qTOF, 19 proteins were identified, with Cthrc1 peptides predominating the mass spectra, followed by various immunoglobulin chains, indicating that the approach was working as intended.

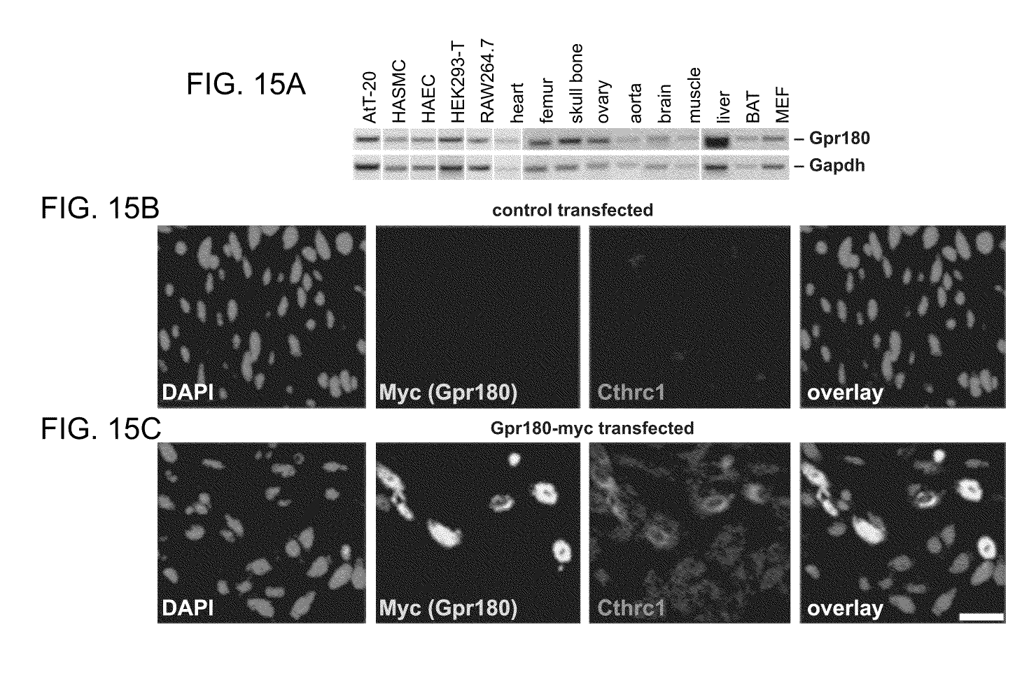

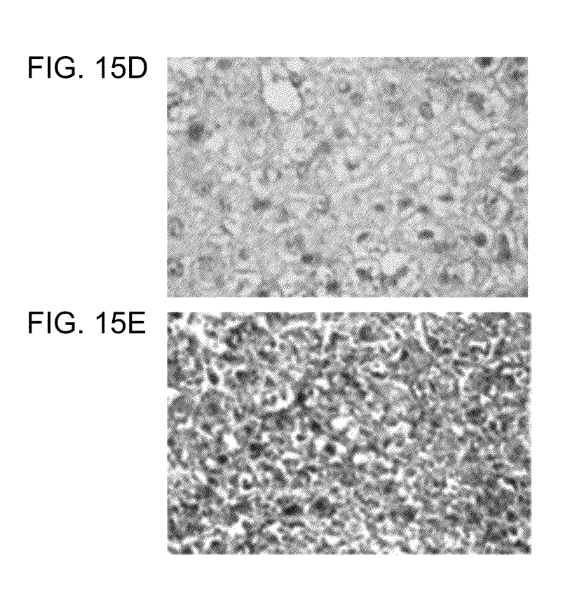

Following the list of immunoglobulins, the integral membrane protein GPR180 was identified with >45% peptide sequence coverage. GPR180 is a G-protein-coupled receptor with typical 7 transmembrane domains. GPR180 (originally termed ITR; intimal thickness related receptor) was identified by Tsukada et al. in 2003 by differential-display analysis of balloon injured rabbit aortae⁸. Similarly, Cthrc1 was identified in balloon-injured rat carotid arteries in a screen for differentially expressed genes⁴. Moreover, GPR180 knockout mice develop normally and exhibit resistance to neointima formation in the femoral artery cuff model⁸. No ligand binding GPR180 has been identified, rendering this protein an orphan GPCR. GPR180 mRNA was widely expressed in many tissues and cell lines (FIG. 15A). No endogenous GPR180 expression was identified in CHO-K1 cells. Receptor binding studies were performed in these cells after transfecting them with a tagged GPR180 expression vector (FIG. 15B and FIG. 15C). Of note, CHO-K1 cells do not express any endogenous Cthrc1, nor do they express detectable levels of GPR180. Furthermore, the quality and sensitivity of the anti-Cthrc1 monoclonal antibodies allows detection of Cthrc1 bound to cells by immunofluorescence. When adding Cthrc1 to control transfected CHO-K1 cells, Cthrc1 bound to cells or matrix was not detected (FIG. 15B). However, when Cthrc1 is added to cells previously transfected with myc-tagged GPR180, complete (100%) overlap of GPR180 cell surface expression with localization of Cthrc1 was observed, providing convincing evidence that the two proteins interact (FIG. 15C). Thus, provided herein is evidence that GPR180 is a Cthrc1 receptor using two separate methodologies, i.e. mass-spectrometry and co-localization that only occurs when both ligand and receptor are present.

In summary, identified herein is a factor with hormone-like functions and its putative receptor, GPR180. Also, Cthrc1 was identified as a factor regulating body composition and lipid metabolism. Described herein are results demonstrating that atherosclerosis is dramatically reduced in the absence of Cthrc1, identifying Cthrc1 and its receptor as treatment targets. Unique monoclonal antibodies were generated for the detection of Cthrc1 in various applications. The sandwich ELISAs developed herein is the only assay used to measure Cthrc1 plasma levels in humans and rodents. These assays are critical for the evaluation of Cthrc1 as a biomarker. Also described herein is evidence that levels of Cthrc1 are increased in diabetes and inflammatory conditions. Cthrc1 is the first ligand identified for the orphan receptor, GPR180. As described herein, antagonists are useful in the treatment of dyslipidemias and atheroslcerosis. Likewise, as described herein, agonists increase muscle mass, which likely has consequences beyond the treatment of diseases associated with decreased muscle strength. Finally, Cre-inducible transgenic mouse strains on the wildtype as well as on the Cthrc1 null background were developed herein. Using tamoxifen inducible albumin-Cre mice, circulating Cthrc1 levels were regulated. In addition, using Pdgfrb-Cre mice, Cthrc1 is selectively re-expressed in activated fibroblast and stromal cells to examine the differential effects of circulating versus locally expressed Cthrc1 in the development of atherosclerosis. Compound mutant mice, Cthrc1 null mice on the ApoE null background were also generated herein.

### Rationale and Conceptual Considerations

In the late 1990's a series of studies were published predicting that Pparγ would promote atherosclerosis. This was based on findings of Pparγ expression in monocyte/macrophages, Pparγ ligands being able to increase expression of fatty acid transporter CD36 and promoting foam cell formation as well as Pparγ being expressed in atherosclerotic lesions. Furthermore, oxidized LDL particles as endogenous Pparγ ligands were thought to promote a vicious cycle characterized by Pparγ activation leading to increased CD36 expression with increased lipid uptake and hence accelerated atheroslcerosis⁹⁻¹². It came as quite a surprise then that Pparγ agonists (thiazolidinedione, TZDs) slowed development and progression of atherosclerosis in animal models³ and humans with type II diabetes (T2DM)^{13, 14}. It was later suggested that the beneficial effects of TZDs on atherosclerosis may at least in part be mediated by increasing HDL-cholesterol^{13, 14}. In addition, a more recent study suggested that the TZDs inhibit atherosclerosis progression independent of their effects on hyperglycemia, insulin resistance and dyslipidemia, indicating direct actions of the TZDs on the vessel wall. Combined, these studies show that prior to the invention described herein, the mechanism of atheroprotective effects of Pparγ was unclear.

Described herein is the evaluation of the role of Cthrc1 on inhibitory effects on Pparγ. As described in detail below, the absence of Cthrc1 in mice is associated with significantly increased expression of Pparγ and its target genes. Furthermore, Cthrc1 null mice on an atherosclerosis prone ApoE null background have lower total cholesterol and increased HDL-cholesterol levels. In addition, patients with T2DM have significantly increased plasma levels of Cthrc1. Finally, absence of Cthrc1 is associated with low bone mass¹⁵ (confirmed in mice herein) and increased body fat (described herein), both known side effects of TZD treatments^{16, 17}. The effects of TZDs on bone and adipose tissue^{2, 18} are caused by critical functions of Pparγ in osteoclast¹⁷ and adipocyte differentiation¹⁹.

Anti-inflammatory and insulin-sensitizing functions of Pparγ agonists have been well established^{20, 21}. However, this does not address potential effects of Cthrc1 on inflammation or glucose metabolism because glucose handling and insulin levels were normal in Cthrc1 null mice⁵. Furthermore, extensive analyses of inflammatory markers (Myriad RBM Panel) in ApoE null mice and ApoE/Cthrc1 double null mice on the high fat diet revealed no differences in C-reactive protein, interleukin family members, IFN-γ, TNF-alpha, MMP-9, soluble VCAM-1 and macrophage inflammatory proteins 1-3. Thus, described herein is lipid metabolism in fat, muscle and liver, not inflammatory state of the vessel wall. With the established roles of Pparγ in lipid metabolism and the findings of altered lipid metabolism in Cthrc1 mice including elevated Pparγ expression, described herein is the mechanism by which Cthrc1 affects lipid metabolism that ultimately affects atherosclerosis.

As described in detail below, Cthrc1 functions as a negative regulator of PPARγ expression, with the absence of Cthrc1 leading to increased expression of this nuclear receptor in cells that express Gpr180. Furthermore, the effects of Cthrc1 on PPARγ levels are at least in part responsible for decreased levels of free fatty acids and increased lipid storage in adipose tissue, which in turn reduces atheroslcerosis and lipid toxicity in muscle²². The latter is supported by finding much lower levels of myoglobin (66 vs. 385 ng/ml), creatine kinase (23 vs. 71U/L), and creatinine (<0.3 vs. 3.6 mg/dL) in serum from ApoE/Cthrc1 double null versus ApoE null mice on a high fat diet.

### Cthrc1 Regulates PPARγ Levels In Vivo

Loss of function analyses in vivo: Currently evidence for a link between Cthrc1 and Pparγ expression in tissues is indirect. Pparγ expression is predominantly expressed in fat tissue. Therefore, first, body composition and size of white fat depots are determined at two different ages in Cthrc1 null vs. wildtype mice, separately for male and female mice (n=10 per group). This is performed in newborn mice (before some of the phenotypes are established), in 10 week old mice, for which there is a set of body composition data, and in 8 month old mice when phenotypes are fully established (e.g. steatosis⁵). NMR (Bruker Minispec) is utilized to determine overall body composition with percentage of body fat and lean mass. The major white adipose depots are quantified in dermis (dorsal skin), inguinal fat pads, intra-abdominal/epididymal depots and brown adipose tissue in the scapular region. After weighing these depots, part of the tissue is used for measuring gene expression levels by qRT-PCR and Western blotting for Pparγ and downstream targets including aP2 (Table 2). In addition, histology is performed on the specimens, average size of the adipocytes is determined, fat storage is visualized with oil red O staining and quantification by image analysis as well as biochemical assays. Because of the increased energy expenditure observed in the Cthrc1 null mice (see below) potential conversion of white adipose (WAT) to brown adipose tissue (BAT, browing) is evaluated by immunohistochemistry and Western blotting for Ucp-1. Browning is also detectable by histology with an increase in cell numbers with typical brown adipocyte morphology²³. Liver (as a major target organ for Cthrc1 determined by binding and steatosis phenotype) and muscle (gastrocnemius) are also included in this analysis. This first set of studies determines whether or not the previously observed increase in Pparγ expression is the result of more body fat (the major source of Pparγ) or whether Pparγ expression is increased in specific tissues. Determination of fat depot mass and average adipocyte size will allow conclusions about adipocyte number and thus effects on adipocyte differentiation.

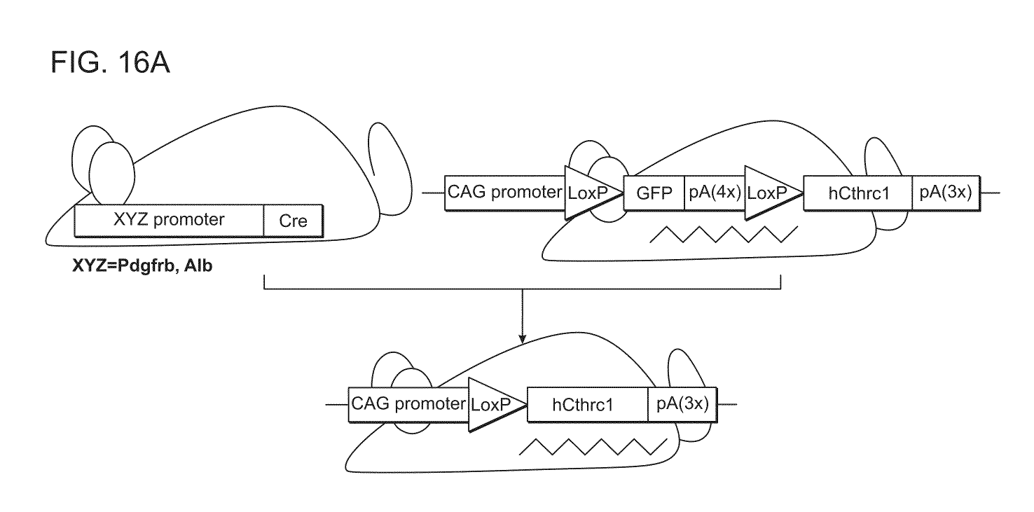

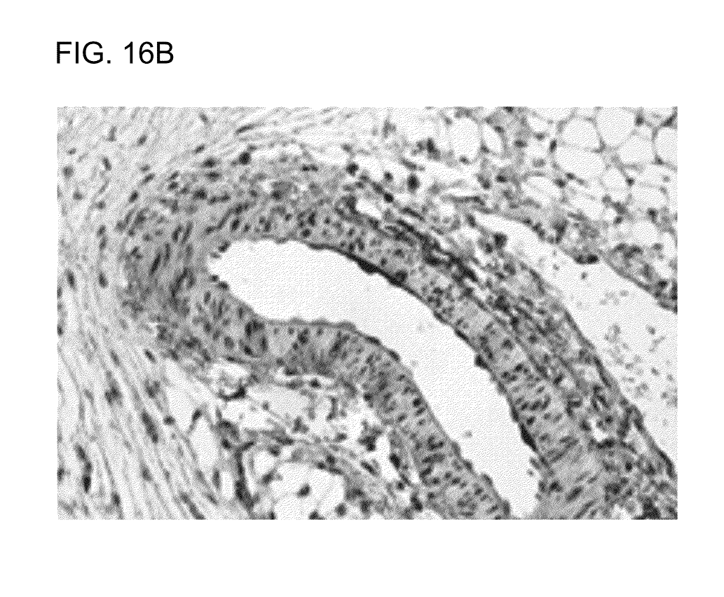

Gain of function studies in vivo: Previous results demonstrated mice that constitutively express a C terminally myc-tagged form of rat Cthrc1 under CMV promoter control⁴. As described herein, C terminal myc tag fused to the transgene likely interfered with Cthrc1 function because none of the C terminally tagged or modified forms of Cthrc1 are detected by ELISA, even though the epitopes recognized by the monoclonal antibodies map to the N terminal half of the molecule. Transgenic mice constitutively expressing wildtype (CMV-hCthrc1) were lethal or the lines could not be maintained. Therefore, strains were generated with inducible human Cthrc1, Tg(CAG-GFP-hCthrc1)^{Vli}, allowing for discrimination between endogenous mouse and transgenic human Cthrc1 (FIG. 16A). Human and mouse Cthrc1 protein are 98% identical. Thus, lack of activity across species is not a concern. Similar inducible transgenics expressing rat Cthrc1 are in hand.

After breeding Cre mouse lines of choice onto the Cthrc1 null background and crossing them with Tg(CAG-GFP-hCthrc1)^{Vli}/FVB-Cthrc1^{tm1Vli} Cthrc1 expression is direct to specific tissues in the absence of any endogenous Cthrc1 expression. Endogenous Cthrc1 is expressed in interstitial, mesenchymal cells of remodeling tissues, which are also known to express Pdgfrb⁵′ ²⁴. As shown in FIG. 16B, crossing Tg(CAG-GFP-hCthrc1)^{Vli} with the Pdgfrb-Cre strain results in Cthrc1 expression in medial SMC as well as the adventitia; however, human Cthrc1 was not detectable in the plasma in 12 out of 14 of these mice despite expression in the vasculature and muscle. In one mouse hCthrc1 levels were at 4.4 ng/ml and this mouse had much reduced body fat (2%) and increased lean mass (81%), and had an average of 16% body fat. Expression in liver results in circulating levels of Cthrc1⁵. Thus, the tamoxifen inducible Cre transgenic mice under control of the albumin promoter (Albtm1(cre/ERT2)^{Mtz}) is utilized. In this mouse, the cre/ERT2 is knocked into the 3′UTR of the albumin gene, resulting in highly efficient induction of Cre with tamoxifen²⁵. This allows precise titering of transgene expression by varying the tamoxifen dose (administered after weaning). The optimal dose for this experiment is determined by monitoring hCthrc1 plasma levels. Littermates negative for Cre will serve as controls and analyses of fat depots and Pparγ expression is performed as described for Cthrc1 null mice above. Mice are examined 2 weeks after tamoxifen injection and at 10 weeks of age (7 weeks after injection).

### Cthrc1 Regulates PPARγ Activity

In vitro studies using a PPARγ reporter and 3T3-L1 differentiation assay: The PPRE X3-TK-luc reporter is a readout of PPARγ activity, and it is transfected into 3T3-L1 cells following an established differentiation protocol (IBMX, dexamethasone, rosiglitazone) into adipocytes. A standard dual luciferase assay with Renilla-luc is used after incubating reporter-transfected cells for various lengths (2, 4, 8, 24 hours) with 100 ng/ml of Cthrc1 (prepared from CHO cells). In addition, cells are treated with Cthrc1 (or vehicle) in a similar manner and PPARγ levels are monitored by immunoblotting, so that changes are monitored (even if it is not at the transcriptional level). After verifying that 3T3-L1 cells express Gpr180, it is determined whether transfection of these cells with a Cthrc1 expression construct or addition of Cthrc1 to the medium prevents or delays differentiation of the 3T3-L1 preadipocytes into adipocytes as quantitatively determined by oil red O staining and expression levels of PPARγ, aP2, and CD36 (at the RNA and protein level).

### Cthrc1 Affects Lipid Uptake, Tissue Lipid Content and Lipid Profile

Alterations in Pparγ expression levels affect lipid content tissues and serum. For example, deletion of Pparγ in endothelial cells and bone marrow of mice led to decreased adiposity, but increased free fatty acid (FFA) and triglyceride (TG) levels when these mice were on a high fat diet. As mentioned above, Cthrc1 null mice have reduced triglycerides and increased body fat. Lipid content in skeletal muscle and liver is measured with the established Bligh-Dyer method. Increased lipid is expected in the liver of Cthrc1 null mice, but the same may not be true for muscle tissue²².

FFA and triglycerides are measured in serum samples of overnight fasted wildtype and mutant mice on normal chow and the high fat diet (TG and NEFA kits from Wako). Lipid fractions Analogues to data on tissue-specific deletion of Paprγ, and FFAs and TGs increase in Cthrc1 overexpressing mice and reduced in Cthrc1 null mice (FIG. 14). A lipid challenge test is performed by gavage feeding of olive oil (10 ml/kg) as described²² because the response to this might differ from lipid handling during fed and fasted conditions. Blood is obtained 3, 6, and 9 hours after the challenge for measurements of FFAs, TGs and assessment of lipemia index. Lower peak levels and more rapid clearance of lipids in the null mutants are observed. Lipid fractions (chylomicrons, VLDL, LDL, HDL) are also determined as decreased FFAs and TG levels in null mice might be due to decreased VLDL production or increased activity of lipoprotein lipase (LPL). If chylomicron or VLDL levels are decreased in Cthrc1 mill mice, TG metabolism is examined in more detail. For example, inhibiting LPL activity by Triton infusion (as described²²) allows measurements of VLDL production. LPL activity and hepatic lipase (HL) are determined in post-heparin plasma as described²². To determine the direct effects on fatty acid uptake, in vitro fatty acid uptake studies are performed in 3T3-L1 adipocytes and primary mouse embryonic fibroblasts. Cells are treated with Cthrc1 (100 ng/ml) for 24 h prior to addition of fluorescently labeled long-chain FA (BODIPY-dodecanoic acid). FA uptake is quantified with the QBT-FA assay kit (Molecular Devices). In this experiment it is also determined whether CD36 expression levels (mRNA and protein) and Pparγ levels decreased in response to Cthrc1 treatment because this is the anticipated mechanism by which Cthrc1 might act.

### Cthrc1 Regulates Metabolism

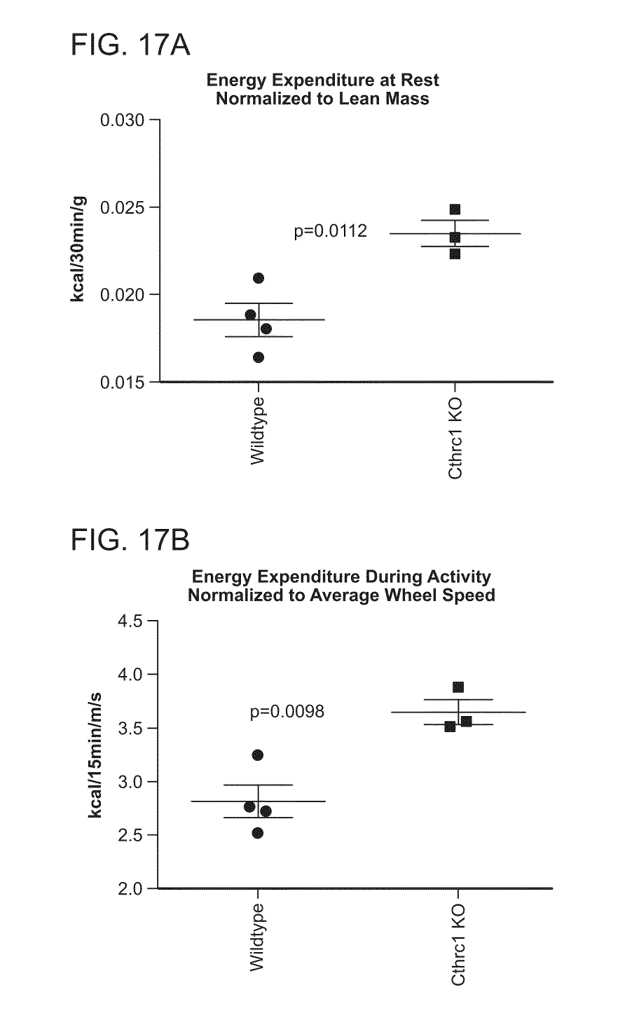

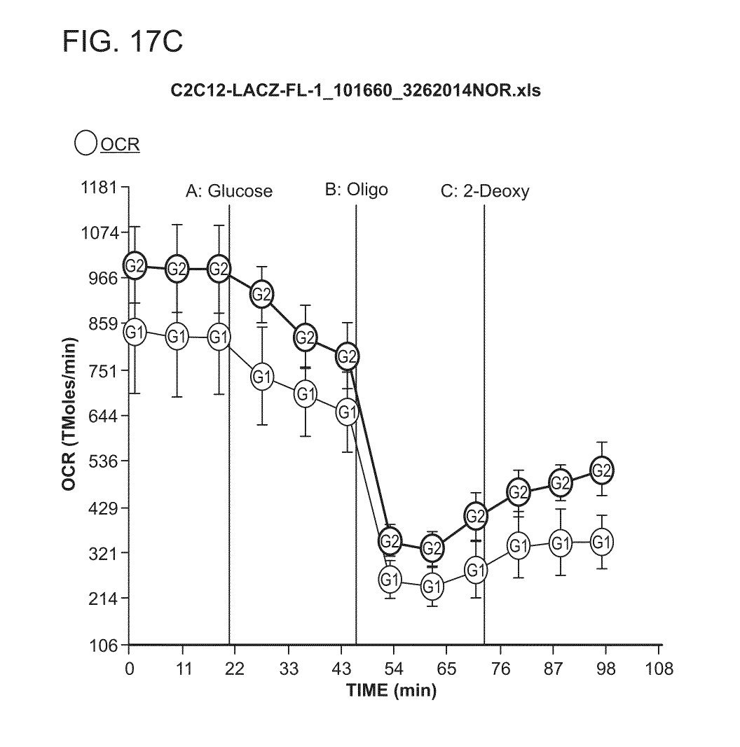

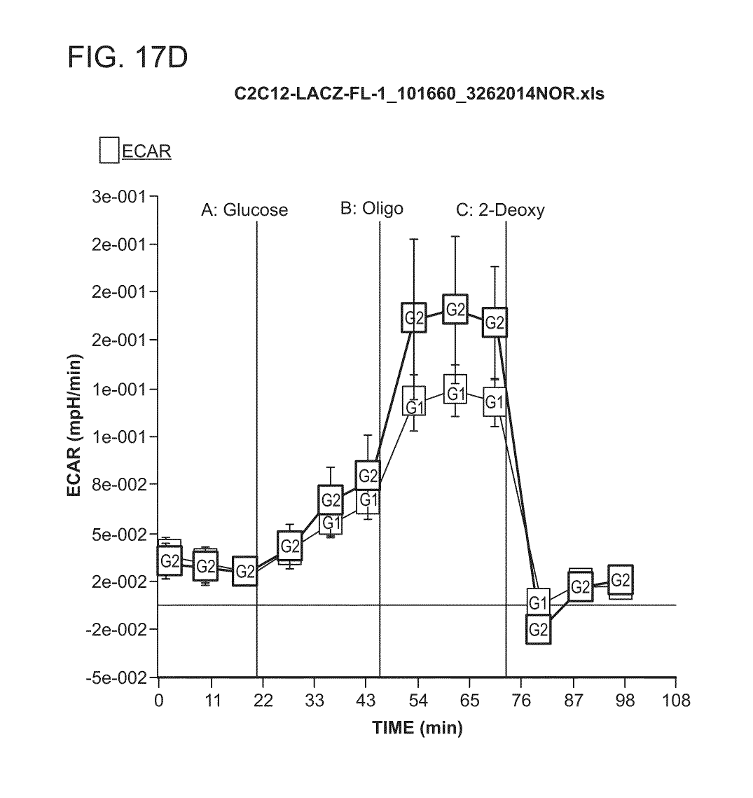

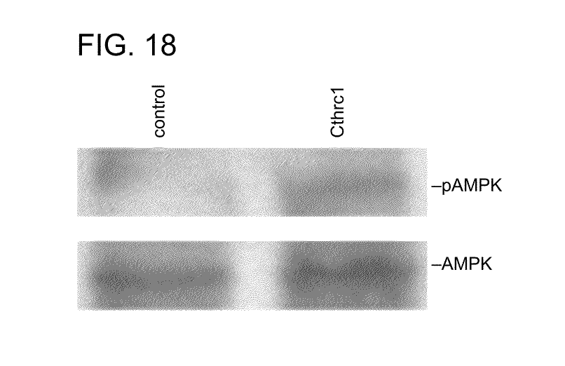

Body composition and size of fat depots is affected by physical activity of the mice. In turn, lean mass and activity have impact on energy expenditure. The potential confounding factor of activity is less likely to play a role in newborn mice because their physical activity and mobility is much more limited. Leptin expression was increased at the RNA level approximately fourfold in 2 day old pups. Because leptin is predominantly expressed in adipose tissue, this may be a sign that Cthrc1 null mice are born with increased fat depots. In the adults, however, it is important that body composition is examined in relation to activity, time spent sleeping, and energy expenditure in the presence of altered Cthrc1 expression levels. The Promethion system acquires complete calorimetry data for day and night time with respect to rest or activity. Activity separately monitors walking, running, time spent still, walking/running speed, distance covered, all with respect to day versus night time. In addition, the system provides data on 3 dimensional directional movement, readouts for activity and rearing behavior. Furthermore, the system has a running wheel that records meters run per day and night, and average wheel speed. Metabolic and activity data was generated for 3 Cthrc1 null and 4 wildtype mice and many results show a trend that are statistically significant with more animals studied per genotype. Energy expenditure at rest is used in determining the basal metabolic rate in mice, referred to here as resting metabolic rate (RMR). This parameter is very important because it provides clues as to the effects of Cthrc1. At rest, energy expenditure is increased in Cthrc1 null compared to wildtype mice (0.461 versus 0.423 kcal/30 min). With muscle/lean mass being a major variable determining RMR, this difference is even more striking (p=0.011) when it is considered that Cthrc1 null mice have significantly less lean mass (Table 1, FIG. 17A-FIG. 17B). In addition, distance covered in the running wheel, the most energy consuming activity, is reduced as well for the Cthrc1 null mice (4739 vs. 6320 m/24 h). Normalizing energy expenditure to average running wheel speed also shows significantly increased energy expenditure in the null mutants (FIG. 17A-FIG. 17B).

Respiratory quotient (RQ) was similar among strains when considering this parameter in relation to activity and time of day. The RQ at rest, both at night and during the day, was similar for both strains if not lower for the wildtypes (0.753 vs. 0.773). The RQ was also similar for both strains during activity at night (0.907 vs. 0.912 for wildtypes and Cthrc1 mutants, respectively). Altogether, this data suggest that in the absence of Cthrc1, energy expenditure is increased. Increasing levels of circulating Cthrc1 in response to stress, physical or metabolic in nature (FIG. 12C) could provide a stimulus to increase efficiency of metabolism, the mechanism to be determined below. It is likely that increased energy expenditure or reduced metabolic efficiency is beneficial for inhibiting atherosclerosis, but it may come at the expense of decreased muscle performance. In-depth metabolic and activity monitoring is performed by including additional Cthrc1 null and wildtype mice as well as gain-of-function mice in these analyses (n=10 per genotype). With the inducible transgene on the Cthrc1 null background, longitudinal studies measuring metabolic and activity parameters are performed before induction of the transgene with tamoxifen (null phenotype) and after induction of the transgene (gain-of-determining the elevation in circulating Cthrc1. Mice with similarly increased circulating levels of Cthrc1 are compared. Standard procedure is data collection over a 5 day period in the metabolic cages.

### Increased Energy Expenditure in the Absence of Cthrc1

Myography data performed on the EDL muscle revealed reduced maximal tetanic force. Considering that EDL mass was reduced in the Cthrc1 null mice, normalizing maximal tetanic force to cross-sectional area of that muscle still showed significantly reduced specific force in the mutant mice (19.19±3.27 vs 22.77±1.48 N/cm², p=0.038). Considering the findings, it is determined whether Cthrc1 null mice produce less ATP necessary for muscle contraction at the expense of more thermogenesis. Thus, examining the expression levels of uncoupling proteins (Ucp, mRNA by qRT-PCR and protein by immunoblotting) is important. Ucp-3 (Slc25A9) is expressed only in skeletal muscle and heart and more so in glycolytic than oxidative skeletal muscle. Ucp-2 (Slc25A8) is expressed in many tissues and Ucp-1 (Slc25A7) is restricted to brown fat. Ucp-2 and -3 expression levels are quantified in the myocardium and separately for glycolytic (EDL) and oxidative (soleus) skeletal muscle. Ucp-2 expression levels are also quantified in adipose tissue and liver. The volume of brown adipose tissue is determined (see above) and Ucp-1 expression examined by immunostaining and qRT-PCR. Fat morphology and ‘browing’ of white fat is observed as described above. These analyses are done in wildtype, Cthrc1 null and overexpressing mice. Increased Ucp expression might be observed in the null mutants and less in the gain-of-function mice. To obtain insight into whether Cthrc1 regulates energy metabolism, oxygen consumption and glycolytic capacity was measured in C2C12 cells stimulated with Cthrc1 (FIG. 17A-FIG. 17B). Cthrc1 increased oxygen consumption and glycolysis, which relates to increased ability to generate ATP. With increased levels of Cthrc1 after exercise (FIG. 12C), this supports the view of Cthrc1 as a stress response factor able to increase production of energy substrates.

### Cthrc1 Regulates Glucose Uptake, Mitochondrial Function and Fatty Acid Metabolism In Vitro

The results in FIG. 17A-FIG. 17B indicate higher OCR in Cthrc1 treated cells. Glucose uptake is measured using [3H]-Deoxy-D-glucose with established glucose uptake assays and quantification of the glucose transporter Glut4 (Slc2a4) levels. Using the Seahorse XF Analyzer (MMCRI Physiology Core), the mitochondrial stress test is performed in C2C12 and 3T3-L1 adipocyte cells in the presence of added Cthrc1 or vehicle. Additional controls for specificity of Cthrc1 effects include cells treated with siRNA knockdown of Gpr180. This test examines whether Cthrc1 affects basic mitochondrial parameters such as ATP turnover, maximal respiration, spare respiratory capacity and proton leak. The latter is of particular interest as it helps clarify whether increased energy expenditure observed in Cthrc1 null mice is caused by uncoupling. Mitochondrial stress tests are also performed.

Based on data described herein obtained in Cthrc1 mice so far, it is likely that Cthrc1 inhibits Pparγ at the RNA and protein level as well as the reporter. The in vitro experiments with the time course data also indicate whether it is with a direct or an indirect effect of Cthrc1 on Pparγ transcription. 3T3-L1 cells likely express Gpr180, as do all mesenchyme-derived cells tested. To provide additional evidence for specificity of Cthrc1 effects and that they require Gpr180, endogenous Grp180 is knocked down with siRNA approaches. Inhibition of the Cthrc1 effects in the siRNA treated cells is additional evidence that Gpr180 is required for Cthrc1 function. If Cthrc1 inhibits the Ppar reporter, this provides an in vitro readout for Cthrc1 activity.

Adiponectin is increased with TZD treatment (Maeda Takahashi Funahashi 2001). The inhibitory effects of AMPK on cholesterol synthesis are mediated via inhibition of Srebp (Srebf1) activity, which in turn is responsible for inhibition of atherosclerosis and hepatic steatosis. Hepatic activation of AMPK by the synthetic polyphenol S17834 protects against hepatic steatosis, hyperlipidemia, and accelerated atherosclerosis in diet-induced insulin resistant LDL receptor deficient mice in part through phosphorylation of SREBP-1c Ser372 and suppression of SREBP-1c and -2-dependent lipogenesis. As described herein, AMPK-dependent phosphorylation of SREBP offers therapeutic strategies to combat insulin resistance, dyslipidemia, and atherosclerosis. Finally, leptin increases FA oxidation via AMPK activation.

### AMP-Activated Protein Kinase (AMPK), Link Between Energy Metabolism and Lipid Metabolism

Cthrc1 is expressed by activated fibroblast-like cells during tissue remodeling, injury and repair. For example, in response to balloon injury or angiotensin II infusion there is extensive proliferation of adventitial cells (Smith et al.²⁶ and FIG. 13C). As described herein, the metabolic needs of highly proliferative cells are higher than those in uninjured vessels. Furthermore, as described herein, in response to physical exercise, Cthrc1 levels rise (FIG. 12C). Putting this into context with increased energy expenditure observed in Cthrc1 null mice, a scenario is envisioned where Cthrc1 expressed locally at sites of tissue remodeling act locally for the duration of the remodeling process to increase metabolic efficiency with the ultimate goal of optimizing repair, and limiting permanent tissue damage, e.g. fibrosis⁴. Cthrc1 expression driven by Pdgfrb-Cre did not result in increased circulating Cthrc1 in most cases. Circulating Cthrc1 with a half-life of 2.5 hours⁵ may act systemically to increase efficiency of skeletal muscle performance under conditions of physical stress.

AMPK is a crucial cellular energy sensor (reviewed in²⁷). Upon activation by decreased ATP levels and energy status, it increases ATP production by elevating the activity or expression of proteins involved in catabolism while at the same time conserving ATP by inhibiting biosynthetic pathways. AMPK is a regulator of metabolic energy homeostasis at the whole-body level with expression in many tissues including liver, skeletal muscle, fat and brain. Activation of AMPK stimulates fatty acid oxidation and glucose uptake while inhibiting lipogenesis, triglyceride synthesis and cholesterol synthesis. Relevant to this is the report²⁸ of synthetic polyphenols that activate AMPK resulting in reduced hepatic steatosis, a condition that does develop in Cthrc1 null mice⁵. Provided herein is evidence for the activation of AMPK by Cthrc1 in FIG. 18.

As described herein, the lack of Cthrc1 leads to inappropriately low levels of activated AMPK at baseline and during conditions when AMPK would otherwise be activated, leading to pathology such as excessive lipid accumulation in the liver. Based on the above consideration, the experiments are performed to determine if pAMPK levels are reduced in Cthrc1 null mice, if transgenic overexpression or injection of Cthrc1 increases pAMPK levels in vivo, and if Cthrc1 activates AMPK in vitro (and, if so, if this effect blocked with Gpr180 siRNA).

As described herein, Cthrc1 effects are mediated by interaction with GPR180. As described in detail below, downstream of this interaction is activation of AMPK and the negative regulation of Pparγ expression and its downstream effectors of lipid metabolism (described above). Reduced activation of AMPK in Cthrc1 null mice in response to stress would be consistent with the altered lipid metabolism described above (for AMPK review see⁴⁹).

### Characterization of Cthrc1 Signaling Through GPR180

### Analysis of GPR180 Activation Following Cthrc1 Binding

It is first determined if binding of Cthrc1 activates GPR180 using the PathHunter β-Arrestin GPCR assay (DiscoverX). This is a cell-based functional assay that directly measures GPCR activity by detecting the interaction of β-Arrestin with the activated GPCR. The assay exploits the fact that β-Arrestin recruitment is independent of G-protein signaling. It is suitable for virtually any Gα-i, Gα-s, or Gα-q—coupled receptor. GPR180 is cloned in frame with the β-gal enzyme fragment (ProLink, enzyme complementation assay). This construct is transfected into CHO cells stably expressing β-Arrestin and the enzyme acceptor deletion mutant of the β-gal enzyme. Upon activation of GPR180, binding of β-Arrestin to the ProLink-tagged GPCR occurs, which forces complementation of the two enzyme fragments with formation of an active β-gal enzyme. This interaction leads to an increase in enzyme activity that is measured using chemiluminescent detection reagents provided with the kit.

Upon receptor activation β-Arrestin-dependent invagination of clathrin coated pits containing receptors and bound ligands may occur along with dynamin-dependent pinching of the pits and formation of endosomes. Ligand and receptor may dissociate in endosomes and the receptor may be recycled to plasma membrane. Alternatively, endosomes undergo maturation and then fuse with lysosomes, where receptor and ligand are degraded. To assess the ability of Cthrc1 to induce internalization of GPR180, CHO cells (no endogenous Cthrc1 receptor) transfected with Gpr180-myc are used. Confocal microscopy is used to localize GPR180 in relation to endosomes and lysosomes (marker antibodies) at different time points after Cthrc1 stimulation. Cthrc1-induced internalization of Gpr180 is also studied in live cells. For that purpose, a GPR180-GFP construct is transfected into CHO cells and observed on the heated stage of the confocal microscope after ligand has been added. The stacks of confocal images of cells with high level of GPR180 are taken immediately before and then every 2 minutes after the stimulation with Cthrc1. The reconstruction of the confocal images is achieved using the Imaris software, resulting in the 3D movie of GPR180 internalization.

To assess the recycling of GPR180, CHO cells transfected with GPR180 tagged with the photoactivatable protein pGFP2 are used. A segment of plasma membrane in a transfected cell is illuminated at 405 nm to activate pGFP2, cells are stimulated with Cthrc1, and trafficking of photoactivated GPR180-pGFP2 is followed. Special attention is given to the potential reappearance of internalized Gpr180 on the cell membrane. To investigate whether Cthrc1 induces GPR180 oligomerization, an HA-tagged GPR180 was generated in addition to the GPR180-myc. CHO cells are cotransfected with an equimolar ratio of GPR180-myc and GPR180-HA constructs followed by stimulation with Cthrc1 (or control vehicle) for 30 sec, 1 min or 2 min. After fixation, cells are co-stained with FITC-conjugated anti-HA and CY3-conjugated anti-myc antibodies. FRET will be assessed.

### Identification of the G-Protein(s) Binding to GPR180

One important step in characterizing a GPR is the identification of its associated G protein(s), because this may contribute to determination of downstream signaling events. An immunoprecipitation approach is used combined with mass-spectrometric analysis of co-immunoprecipitated proteins. CHO cells lacking endogenous GPR180 are stably transduced with a pWZL retroviral construct of myc-tagged GPR180, essentially as previously described⁵⁰. The rabbit monoclonal anti-myc (>15 times more sensitive than mouse anti-myc clone 9E10) is used to immunoprecipitate ligand-receptor-G-protein complexes in a similar manner as was performed for identification of the Cthrc1 receptor (MMCRI proteomics core facility). A minor limitation is that the protein sequence databases for hamster (CHO cells) are not as robust as those for mouse and human. However, the mouse is sufficiently closely related that tryptic peptide fragments may be queried against mouse sequence databases. Alternatively, a murine or human cell line with low endogenous GPR180 levels is used. Co-immunoprecipitated proteins are also immunoblotted with a panel of Ga-protein specific antibodies, both to identify G-proteins binding to GPR180 as well as to verify candidates identified by mass-spectropmetry. Co-localization of identified G-proteins with GPR180 is further assessed using confocal microscopy and fluorescence resonance energy transfer (FRET).

### Cthrc1-Stimulated Signaling Pathways Downstream of GPR180

In response to activation of a GPR, specific signaling pathways get activated. To determine which ones get activated in response to ligand-stimulated GPR180 10 common pathways are examined using the GPCR Signaling 10-Pathway Reporter Array Cignal Finder Reporter Array (Qiagen). This is a cell based reporter assay that measures the functional consequences of GPR activation or inhibition. Among the common pathways are the cAMP/PKA pathway (CREB reporter), calcium/PKC pathway (NFAT reporter), ERK and JNK signaling (ELK1/SRF and FOS/JUN reporter), PI-3 kinase/AKT pathway (FOXO reporter), MEF2 pathway (MEF2 reporter), hedgehog pathway (GLI reporter), NF-κB pathway (NF-κB reporter) and the JAK/STAT pathway (STAT3 reporter). Using these reporter constructs, a series of dual-luciferase assays are performed on CHO cells stably transduced with a GPR180-myc lentiviral vector and stimulated with Cthrc1. This approach reliably identifies the specific signaling pathway affected by GPR180 signaling. Future small molecule screens for GPR180 agonists and antagonists build on this information to establish screening assays.

### Identification of Downstream Targets for GPR180

Activation of AMPK and expression of putative downstream targets of Cthrc1 signaling (CD36, Pparγ, FASN, Slc27a5, Acs15, Acsm3, Cpt1a, Hdac5, Srebp-1, Foxo1, Foxo3) is also evaluated. In vitro studies for Cthrc1 activating AMPK resulting in decreased fatty acid uptake and increased lipolysis are first conducted in 3T3-L1 cells and C2C12 cells as surrogates for metabolically active tissues like fat and muscle. Using these cells is justified because it is likely that decreased atherosclerosis in Cthrc1 null mice is a consequence of increased Pparγ expression and a less atherogenic lipid profile. Of note, Pparγ agonists are athero-protective^{3, 18}. It is also determined whether differentiation of pre-adipocyte 3T3-L1 cells into fat cells is inhibited by Cthrc1 and whether this is paralleled by downregulation of Pparγ expression (assays as described⁵¹). The effects of Cthrc1 on fatty acid uptake is determined with standard assays as described⁵² using 3T3-L1 cells, primary human aortic smooth muscle and endothelial cells as well as macrophages (RAW264.7 cells and mouse alveolar macrophages). Visualization of fatty acid uptake is done with fluorescent BODIPY-C12/C16.³H-oleic acid is used to measure lipid uptake quantitatively in vitro. Effects on lipolysis in vivo as wells in vitro is determined as described^{51, 53}. The in vivo studies are done in wildtype, Cthrc1 null, and Cthrc1 transgenic mice on the Cthrc1 knockout background with and without induction of the transgene (n=5 mice per group). The selective β3-adrenergic agonist BRL37344 are injected (i.p., 5 μg/g) and blood will be obtained at 0, 10, and 20 min after injection for measurements of serum free glycerol as an indicator of lipolysis (kit from Sigma-Aldrich). Because of potential desensitization with prolonged exposure in the transgenic approach, an additional experiment is also performed where Cthrc1 (purified from transduced CHO cells) or vehicle is administered to Cthrc1 null mice acutely by intravenous injection. In one aspect, lipolysis is decreased in Cthrc1 null and increased in mice with elevated Cthrc1 levels.

Cthrc1 effects on AMPK activation are also examined in primary human vascular SMC (increase in pAMPK-T¹⁷²). Confirmation that Cthrc1 activates AMPK will lead to a series of follow-up experiments that are presently beyond the scope of this application (effects on hormone sensitive lipase-HSL, PGC-1α, PPAR-α, PPAR-δ. CPT-1b, etc.).

The evaluation of Cthrc1 signaling is described herein. A broader, unbiased approach toward identification of Cthrc1 targets compares changes in the SMC proteome following stimulation by Cthrc1 or vehicle by mass-spectrometry as previously implemented^{54, 55}. Protein subfractions are labeled with light (control) and heavy (Cthrc1) ICAT 28 reagents, LC-MSMS (QSTAR, 4000QTRAP) is conducted to quantify changes in protein expression, as measured by the Heavy/Light isotope ratio, and the proteins are identified. High priority candidates for further analysis are identified based on functional criteria, known involvement in vascular disease or lipid metabolism. Confirmation of Cthrc1 regulated proteins is accomplished by quantitative real time RT-PCR and immunoblotting of important candidate proteins. Cthrc1 as a hormonal activator of AMPK via GPR180 provides a much needed readout for Cthrc1 activity. Note that the much older adiponectin field was lacking a readout for activity for a long time and only recently identified activation of ceramidase as a suitable parameter for adiponectin activity. Finally, a GPR180 null mouse is generated using the time-saving Crisper/Cas9 technology (SAGE Laboratories)^{8.}

### REFERENCES CITED

1. Shepherd J, Cobbe S M, Ford I, Isles C G, Lorimer A R, MacFarlane P W, McKillop J H, Packard C J. Prevention of coronary heart disease with pravastatin in men with hypercholesterolemia. West of Scotland Coronary Prevention Study Group. N Engl J Med. 1995; 333:1301-1307.
2. Saremi A, Schwenke D C, Buchanan T A, Hodis H N, Mack W J, Banerji M, Bray G A, Clement S C, Henry R R, Kitabchi A E, Mudaliar S, Ratner R E, Stentz F B, Musi N, Tripathy D, DeFronzo R A, Reaven P D. Pioglitazone slows progression of atherosclerosis in prediabetes independent of changes in cardiovascular risk factors. Arterioscler Thromb Vasc Biol. 2013; 33:393-399.
3. Li A C, Brown K K, Silvestre M J, Willson T M, Palinski W, Glass C K. Peroxisome proliferator-activated receptor gamma ligands inhibit development of atherosclerosis in LDL receptor-deficient mice. J Clin Invest. 2000; 106:523-531.
4. Pyagay P, Heroult M, Wang Q, Lehnert W, Belden J, Liaw L, Friesel R E, Lindner V. Collagen triple helix repeat containing 1, a novel secreted protein in injured and diseased arteries, inhibits collagen expression and promotes cell migration. Circ Res. 2005; 96:261-268.
5. Stohn J P, Perreault N G, Wang Q, Liaw L, Lindner V. Cthrc1, a novel circulating hormone regulating metabolism. PLoS One. 2012; 7:e47142.
6. Son N H, Park T S, Yamashita H, Yokoyama M, Huggins L A, Okajima K, Homma S, Szabolcs M J, Huang L S, Goldberg U. Cardiomyocyte expression of PPARgamma leads to cardiac dysfunction in mice. J Clin Invest. 2007; 117:2791-2801.
7. Maeda N, Takahashi M, Funahashi T, Kihara S, Nishizawa H, Kishida K, Nagaretani H, Matsuda M, Komuro R, Ouchi N, Kuriyama H, Hotta K, Nakamura T, Shimomura I, Matsuzawa Y. PPARgamma ligands increase expression and plasma concentrations of adiponectin, an adipose-derived protein. Diabetes. 2001; 50:2094-2099.
8. Tsukada S, Iwai M, Nishiu J, Itoh M, Tomoike H, Horiuchi M, Nakamura Y, Tanaka T. Inhibition of experimental intimal thickening in mice lacking a novel G-protein-coupled receptor. Circulation. 2003; 107:313-319.
9. Tontonoz P, Nagy L, Alvarez J G, Thomazy V A, Evans R M. PPARgamma promotes monocyte/macrophage differentiation and uptake of oxidized LDL. Cell. 1998; 93:241-252.
10. Nagy L, Tontonoz P, Alvarez J G, Chen H, Evans R M. Oxidized LDL regulates macrophage gene expression through ligand activation of PPARgamma. Cell. 1998; 93:229-240.
11. Ricote M, Huang J, Fajas L, Li A, Welch J, Najib J, Witztum J L, Auwerx J, Palinski W, Glass C K. Expression of the peroxisome proliferator-activated receptor gamma (PPARgamma) in human atherosclerosis and regulation in macrophages by colony stimulating factors and oxidized low density lipoprotein. Proc Natl Acad Sci USA. 1998; 95:7614-7619.
12. Marx N, Sukhova G, Murphy C, Libby P, Plutzky J. Macrophages in human atheroma contain PPARgamma: differentiation-dependent peroxisomal proliferator-activated receptor gamma(PPARgamma) expression and reduction of MMP-9 activity through PPARgamma activation in mononuclear phagocytes in vitro. Am J Pathol. 1998; 153:17-23.
13. Mazzone T, Meyer P M, Feinstein S B, Davidson M H, Kondos G T, D'Agostino R B, Sr., Perez A, Provost J C, Haffner S M. Effect of pioglitazone compared with glimepiride on carotid intima-media thickness in type 2 diabetes: a randomized trial. JAMA. 2006; 296:2572-2581.
14. Davidson M, Meyer P M, Haffner S, Feinstein S, D'Agostino R, Sr., Kondos G T, Perez A, Chen Z, Mazzone T. Increased high-density lipoprotein cholesterol predicts the pioglitazone-mediated reduction of carotid intima-media thickness progression in patients with type 2 diabetes mellitus. Circulation. 2008; 117:2123-2130.
15. Kimura H, Kwan K M, Zhang Z, Deng J M, Darnay B G, Behringer R R, Nakamura T, de Crombrugghe B, Akiyama H. Cthrc1 is a positive regulator of osteoblastic bone formation. PLoS ONE. 2008; 3:e3174.
16. Habib Z A, Haystad S L, Wells K, Divine G, Pladevall M, Williams L K. Thiazolidinedione use and the longitudinal risk of fractures in patients with type 2 diabetes mellitus. J Clin Endocrinol Metab. 2010; 95:592-600.
17. Wei W, Zeve D, Wang X, Du Y, Tang W, Dechow P C, Graff J M, Wan Y. Osteoclast progenitors reside in the peroxisome proliferator-activated receptor gamma-expressing bone marrow cell population. Mol Cell Biol. 2011; 31:4692-4705.
18. Ahmadian M, Suh J M, Hah N, Liddle C, Atkins A R, Downes M, Evans R M. PPARgamma signaling and metabolism: the good, the bad and the future. Nat Med. 2013; 19:557-566.
19. Tang W, Zeve D, Seo J, Jo A Y, Graff J M. Thiazolidinediones regulate adipose lineage dynamics. Cell Metab. 2011; 14:116-122.
20. Olefsky J M. Treatment of insulin resistance with peroxisome proliferator-activated receptor gamma agonists. J Clin Invest. 2000; 106:467-472.
21. Orasanu G, Ziouzenkova O, Devchand P R, Nehra V, Hamdy O, Horton E S, Plutzky J. The peroxisome proliferator-activated receptor-gamma agonist pioglitazone represses inflammation in a peroxisome proliferator-activated receptor-alpha-dependent manner in vitro and in vivo in mice. J Am Coll Cardiol. 2008; 52:869-881.
22. Kanda T, Brown J D, Orasanu G, Vogel S, Gonzalez F J, Sartoretto J, Michel T, Plutzky J. PPARgamma in the endothelium regulates metabolic responses to high-fat diet in mice. J Clin Invest. 2009; 119:110-124.
23. Hallakou S, Doare L, Foufelle F, Kergoat M, Guerre-Millo M, Berthault M F, Dugail I, Morin J, Auwerx J, Ferre P. Pioglitazone induces in vivo adipocyte differentiation in the obese Zucker fa/fa rat. Diabetes. 1997; 46:1393-1399.
24. Cuttler A S, Leclair R J, Stohn J P, Wang Q, Sorenson C M, Liaw L, Lindner V. Characterization of pdgfrb-cre transgenic mice reveals reduction of ROSA26 reporter activity in remodeling arteries. Genesis. 2011.
25. Schuler M, Dierich A, Chambon P, Metzger D. Efficient temporally controlled targeted somatic mutagenesis in hepatocytes of the mouse. Genesis. 2004; 39:167-172.
26. Smith J D, Bryant S R, Couper L L, Vary C P, Gotwals P J, Koteliansky V E, Lindner V. Soluble transforming growth factor-beta type II receptor inhibits negative remodeling, fibroblast transdifferentiation, and intimal lesion formation but not endothelial growth. Circ Res. 1999; 84:1212-1222.
27. Hardie D G, Ross F A, Hawley S A. AMPK: a nutrient and energy sensor that maintains energy homeostasis. Nat Rev Mol Cell Biol. 2012; 13:251-262.
28. Li Y, Xu S, Mihaylova M M, Zheng B, Hou X, Jiang B, Park O, Luo Z, Lefai E, Shyy J Y, Gao B, Wierzbicki M, Verbeuren T J, Shaw R J, Cohen R A, Zang M. AMPK phosphorylates and inhibits SREBP activity to attenuate hepatic steatosis and atherosclerosis in diet-induced insulin-resistant mice. Cell Metab. 2011; 13:376-388.
29. Kumar A, Lindner V. Remodeling with neointima formation in the mouse carotid artery after cessation of blood flow. Arterioscler Thromb Vasc Biol. 1997; 17:2238-2244.
30. Lindner V, Fingerle J, Reidy M A. Mouse model of arterial injury. Circ Res. 1993; 73:792-796.
31. Slominski A, Tobin D J, Shibahara S, Wortsman J. Melanin pigmentation in mammalian skin and its hormonal regulation. Physiol Rev. 2004; 84:1155-1228.
32. Cabrero A, Alegret M, Sanchez R M, Adzet T, Laguna J C, Vazquez M. Bezafibrate reduces mRNA levels of adipocyte markers and increases fatty acid oxidation in primary culture of adipocytes. Diabetes. 2001; 50:1883-1890.
33. Han X, Gross R W. Shotgun lipidomics: electrospray ionization mass spectrometric analysis and quantitation of cellular lipidomes directly from crude extracts of biological samples. Mass Spectrom Rev. 2005; 24:367-412.
34. Han X, Yang J, Cheng H, Yang K, Abendschein D R, Gross R W. Shotgun lipidomics identifies cardiolipin depletion in diabetic myocardium linking altered substrate utilization with mitochondrial dysfunction. Biochemistry. 2005; 44:16684-16694.
35. Han X, Yang K, Cheng H, Fikes K N, Gross R W. Shotgun lipidomics of phosphoethanolamine-containing lipids in biological samples after one-step in situ derivatization. J Lipid Res. 2005; 46:1548-1560.
36. Han X, Yang K, Gross R W. Multi-dimensional mass spectrometry-based shotgun lipidomics and novel strategies for lipidomic analyses. Mass Spectrom Rev. 2012; 31:134-178.
37. Chiu H C, Kovacs A, Blanton R M, Han X, Courtois M, Weinheimer C J, Yamada K A, Brunet S, Xu H, Nerbonne J M, Welch M J, Fettig N M, Sharp T L, Sambandam N, Olson K M, Ory D S, Schaffer J E. Transgenic expression of fatty acid transport protein 1 in the heart causes lipotoxic cardiomyopathy. Circ Res. 2005; 96:225-233.
38. Yin W, Mu J, Birnbaum M J. Role of AMP-activated protein kinase in cyclic AMP-dependent lipolysis In 3T3-L1 adipocytes. J Biol Chem. 2003; 278:43074-43080.
39. Koh H J, Hirshman M F, He H, Li Y, Manabe Y, Balschi J A, Goodyear L J. Adrenaline is a critical mediator of acute exercise-induced AMP-activated protein kinase activation in adipocytes. Biochem J. 2007; 403:473-481.
40. Rubin L J, Magliola L, Feng X, Jones A W, Hale C C. Metabolic activation of AMP kinase in vascular smooth muscle. J Appl Physiol (1985). 2005; 98:296-306.
41. Liang Y, Huang B, Song E, Bai B, Wang Y. Constitutive activation of AMPK alpha1 in vascular endothelium promotes high-fat diet-induced fatty liver injury: role of COX-2 induction. Br J Pharmacol. 2014; 171:498-508.
42. Jackson I J, Budd P S, Keighren M, McKie L. Humanized MC1R transgenic mice reveal human specific receptor function. Hum Mol Genet. 2007; 16:2341-2348.
43. Slominski A, Plonka P M, Pisarchik A, Smart J L, Tolle V, Wortsman J, Low M J. Preservation of eumelanin hair pigmentation in proopiomelanocortin-deficient mice on a nonagouti (a/a) genetic background. Endocrinology. 2005; 146:1245-1253.
44. Krude H, Biebermann H, Luck W, Horn R, Brabant G, Gruters A. Severe early-onset obesity, adrenal insufficiency and red hair pigmentation caused by POMC mutations in humans. Nat Genet. 1998; 19:155-157.
45. Mountjoy K G. The human melanocyte stimulating hormone receptor has evolved to become “super-sensitive” to melanocortin peptides. Mol Cell Endocrinol. 1994; 102:R7-11.
46. Wikberg J E, Muceniece R, Mandrika I, Prusis P, Lindblom J, Post C, Skottner A. New aspects on the melanocortins and their receptors. Pharmacol Res. 2000; 42:393-420.
47. Begriche K, Girardet C, McDonald P, Butler A A. Melanocortin-3 receptors and metabolic homeostasis. Prog Mol Biol Transl Sci. 2013; 114:109-146.
48. Girardet C, Butler A A. Neural melanocortin receptors in obesity and related metabolic disorders. Biochim Biophys Acta. 2014; 1842:482-494.
49. Gaidhu M P, Ceddia R B. The role of adenosine monophosphate kinase in remodeling white adipose tissue metabolism. Exerc Sport Sci Rev. 2011; 39:102-108.
50. Conley B A, Koleva R, Smith J D, Kacer D, Zhang D, Bernabeu C, Vary C P. Endoglin controls cell migration and composition of focal adhesions: function of the cytosolic domain. J Biol Chem. 2004; 279:27440-27449.
51. Qiao L, Kinney B, Schaack J, Shao J. Adiponectin inhibits lipolysis in mouse adipocytes. Diabetes. 2011; 60:1519-1527.
52. Stremmel W, Strohmeyer G, Berk P D. Hepatocellular uptake of oleate is energy dependent, sodium linked, and inhibited by an antibody to a hepatocyte plasma membrane fatty acid binding protein. Proc Natl Acad Sci USA. 1986; 83:3584-3588.
53. Kinney B P, Qiao L, Levaugh J M, Shao J. B56alpha/protein phosphatase 2A inhibits adipose lipolysis in high-fat diet-induced obese mice. Endocrinology. 2010; 151:3624-3632.
54. Romero D, O'Neill C, Terzic A, Contois L, Young K, Conley B A, Bergan R C, Brooks P C, Vary C P. Endoglin regulates cancer-stromal cell interactions in prostate tumors. Cancer Res. 2011; 71:3482-3493.
55. Young K, Conley B, Romero D, Tweedie E, O'Neill C, Pinz I, Brogan L, Lindner V, Liaw L, Vary C P. BMP9 regulates endoglin-dependent chemokine responses in endothelial cells. Blood. 2012; 120:4263-4273.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating or preventing steatosis or a steatosis-associated condition in a subject, the method comprising:
identifying a subject having or at risk of developing steatosis or a steatosis-associated condition;
administering to the subject an effective amount of a Collagen Triple Helix Repeat Containing 1 (Cthrc1) polypeptide or a Cthrc1 receptor agonist, thereby treating or preventing steatosis or a steatosis-associated condition in the subject.

2. The method of claim 1, wherein said steatosis or a steatosis-associated condition is selected from the group consisting of hepatic steatosis and cardiac steatosis.

3. The method of claim 1, wherein the composition is administered systemically.

4. The method of claim 1, wherein the effective amount of Cthrc1 polypeptide or Cthrc1 receptor agonist is sufficient to reduce lipids in target organs by at least 5%.

5. The method of claim 1, wherein the Cthrc1 receptor comprises G Protein-Coupled Receptor 180 (GPR180).

6. A method of treating or preventing low bone mass or a low bone mass-associated condition in a subject, the method comprising:
identifying a subject having or at risk of developing low bone mass or a low bone mass-associated condition;
administering to the subject an effective amount of a Cthrc1 polypeptide or a Cthrc1 receptor agonist, thereby treating or preventing low bone mass or a low bone mass-associated condition in the subject.

7. The method of claim 6, wherein the low bone mass or low bone mass-associated condition comprises osteoporosis.

8. The method of claim 6, wherein the composition is administered systemically.

9. The method of claim 6, wherein the effective amount of Cthrc1 receptor agonist is sufficient to increase bone mass by at least 5%.

10. The method of claim 1, wherein the Cthrc1 receptor comprises GPR180.

11. A method of treating or preventing muscle weakness or a muscle weakness-associated condition in a subject, the method comprising:
identifying a subject having or at risk of developing muscle weakness or a muscle weakness-associated condition;
administering to the subject an effective amount of a Cthrc1 polypeptide or a Cthrc1 receptor agonist, thereby treating or preventing muscle weakness or a muscle weakness-associated condition in the subject.

12. The method of claim 11, wherein the muscle weakness or a muscle weakness-associated condition comprises muscular dystrophy or age-related frailty and muscle weakness.

13. The method of claim 11, wherein the composition is administered systemically.

14. The method of claim 11, wherein the effective amount of Cthrc1 receptor agonist is sufficient to increase muscle strength by at least 5%.

15. The method of claim 11, wherein the Cthrc1 receptor comprises GPR180.

16. The method of claim 11, wherein said effective amount of Cthrc1 polypeptide or Cthrc1 receptor agonist activates adenosine monophosphate-activated protein kinase (AMPK).

17. A method for identifying a subject having low bone bass, the method comprising:
obtaining a biological sample from said subject;
determining the level of a CTHRC1 polypeptide in a biological sample from the subject;
comparing the level of a CTHRC1 polypeptide in a biological sample from the subject to a normal control level of a CTHRC1 polypeptide, wherein a reduced level of CTHRC1 relative to the normal control level is indicative of the subject having low bone mass.

18. A method for identifying a subject having muscle weakness, the method comprising:
obtaining a biological sample from said subject;
determining the level of a CTHRC1 polypeptide in a biological sample from the subject;
comparing the level of a CTHRC1 polypeptide in a biological sample from the subject to a normal control level of a CTHRC1 polypeptide, wherein a reduced level of CTHRC1 relative to the normal control level is indicative of the subject having muscle weakness.

19. The method of claim 18, wherein said muscle weakness comprises reduced strength of fast-twitch muscle fibers.

20. A method of treating or preventing metabolic syndrome or a metabolic syndrome-associated condition in a subject, the method comprising:
identifying a subject having or at risk of developing metabolic syndrome or a metabolic syndrome-associated condition;
administering to the subject an effective amount of a Cthrc1 polypeptide or a Cthrc1 receptor agonist, thereby treating or preventing metabolic syndrome or a metabolic syndrome-associated condition in the subject.

21. The method of claim 1, wherein the Cthrc1 receptor comprises G Protein-Coupled Receptor 180 (GPR180).

22. A method of treating or preventing atherosclerosis or an atherosclerosis-associated condition in a subject, the method comprising:
identifying a subject having or at risk of developing atherosclerosis or an atherosclerosis-associated condition;
administering to the subject an effective amount of a Collagen Triple Helix Repeat Containing 1 (Cthrc1) polypeptide antagonist or a Cthrc1 receptor antagonist, thereby treating or preventing steatosis or a steatosis-associated condition in the subject.

23. The method of claim 22, wherein said Cthrc1 receptor comprises G Protein-Coupled Receptor 180 (GPR180).

24. A method of treating or preventing obesity or an obesity-associated condition in a subject, the method comprising:
identifying a subject having or at risk of developing atherosclerosis or an atherosclerosis-associated condition;
administering to the subject an effective amount of a Collagen Triple Helix Repeat Containing 1 (Cthrc1) polypeptide antagonist or a Cthrc1 receptor antagonist, thereby treating or preventing obesity or an obesity-associated condition in the subject.

25. The method of claim 24, wherein said Cthrc1 receptor comprises G Protein-Coupled Receptor 180 (GPR180).

26. The method of claim 24, wherein the obesity-associate condition is diabetes.


SEQUENCE LISTING (14 sequences)

SEQ ID NO: 1 - DNA, 25 bp, Artificial Sequence
  GPR180 primer 1
ggagcagtttgaagacaccagtcac

SEQ ID NO: 2 - DNA, 22 bp, Artificial Sequence
  GPR180 Primer 2
tggcacaagaaccacaggatgc

SEQ ID NO: 3 - DNA, 22 bp, Artificial Sequence
  1. GAPDH primer 1
atcactgccacccagaagactg

SEQ ID NO: 4 - DNA, 20 bp, Artificial Sequence
  2. GAPDH primer 2
atccacgacggacacattgg

SEQ ID NO: 5 - protein, 16 aa, Artificial Sequence
  5. human Cthrc1 N terminus
SENPKVKQKAQLRQRE

SEQ ID NO: 6 - protein, 16 aa, Artificial Sequence
  6. murine Cthrc1 N terminus
SENPKVKQKALIRQRE

SEQ ID NO: 7 - protein, 243 aa, Homo sapiens [sequence omitted]

SEQ ID NO: 8 - DNA, 1279 bp, Homo sapiens
gggagggagagaggcgcgcgggtgaaaggcgcattgatgcagcctgcggcggcctcggagcgcggcggagccagacgctgaccacgttcctctcctcggtctcctccgcctccagctccgcgctgcccggcagccgggagccatgcgaccccagggccccgccgcctccccgcagcggctccgcggcctcctgctgctcctgctgctgcagctgcccgcgccgtcgagcgcctctgagatccccaaggggaagcaaaaggcgcagctccggcagagggaggtggtggacctgtataatggaatgtgcttacaagggccagcaggagtgcctggtcgagacgggagccctggggccaatggcattccgggtacacctgggatcccaggtcgggatggattcaaaggagaaaagggggaatgtctgagggaaagctttgaggagtcctggacacccaactacaagcagtgttcatggagttcattgaattatggcatagatcttgggaaaattgcggagtgtacatttacaaagatgcgttcaaatagtgctctaagagttttgttcagtggctcacttcggctaaaatgcagaaatgcatgctgtcagcgttggtatttcacattcaatggagctgaatgttcaggacctcttcccattgaagctataatttatttggaccaaggaagccctgaaatgaattcaacaattaatattcatcgcacttcttctgtggaaggactttgtgaaggaattggtgctggattagtggatgttgctatctgggttggtacttgttcagattacccaaaaggagatgcttctactggatggaattcagtttctcgcatcattattgaagaactaccaaaataaatgctttaattttcatttgctacctctttttttattatgccttggaatggttcacttaaatgacattttaaataagtttatgtatacatctgaatgaaaagcaaagctaaatatgtttacagaccaaagtgtgatttcacactgtttttaaatctagcattattcattttgcttcaatcaaaagtggtttcaatattttttttagttggttagaatactttcttcatagtcacattctctcaacctataatttggaatattgttgtggtcttttgttttttctcttagtatagcatttttaaaaaaatataaaagctaccaatctttgtacaatttgtaaatgttaagaattttttttatatctgttaaataaaaattatttccaacaaccttaatatctttaaa

SEQ ID NO: 9 - DNA, 1214 bp, Homo sapiens
agaaggtttaaggccggaaagggaaatgaaggggcccggcgctaaccctctaaggacctgttttgcttctgtttaaaccaaatgggcagtctgtcattacacacaccctgggtcttcatatgtggccgccaggtaggagcatcacagtcaagctacgggagaaaacagtttccaggaaactggaaatgaacggcccgagtgctttccaggggctcatctgtgggaagtataatggaatgtgcttacaagggccagcaggagtgcctggtcgagacgggagccctggggccaatggcattccgggtacacctgggatcccaggtcgggatggattcaaaggagaaaagggggaatgtctgagggaaagctttgaggagtcctggacacccaactacaagcagtgttcatggagttcattgaattatggcatagatcttgggaaaattgcggagtgtacatttacaaagatgcgttcaaatagtgctctaagagttttgttcagtggctcacttcggctaaaatgcagaaatgcatgctgtcagcgttggtatttcacattcaatggagctgaatgttcaggacctcttcccattgaagctataatttatttggaccaaggaagccctgaaatgaattcaacaattaatattcatcgcacttcttctgtggaaggactttgtgaaggaattggtgctggattagtggatgttgctatctgggttggtacttgttcagattacccaaaaggagatgcttctactggatggaattcagtttctcgcatcattattgaagaactaccaaaataaatgctttaattttcatttgctacctctttttttattatgccttggaatggttcacttaaatgacattttaaataagtttatgtatacatctgaatgaaaagcaaagctaaatatgtttacagaccaaagtgtgatttcacactgtttttaaatctagcattattcattttgcttcaatcaaaagtggtttcaatattttttttagttggttagaatactttcttcatagtcacattctctcaacctataatttggaatattgttgtggtcttttgttttttctcttagtatagcatttttaaaaaaatataaaagctaccaatctttgtacaatttgtaaatgttaagaattttttttatatctgttaaataaaaattatttccaacaaccttaatatctttaaa

SEQ ID NO: 10 - DNA, 2877 bp, Mus musculus
ctgctgccgacgtgggctgcaggccgcaggcggttgccgggcgagcaaacggagcgggcggcgggcatgggcggcctgcggctgctggcggtagccctcacgtgcagctgctggtggccgcagggcggccagggcaagaccctgcgtggcagcttcagcagcgccgcggcccgcgacgcccagggccagagcatcggccatttcgagttccacggtgaccatgctcttctgtgtgtcagaatcaacaacgtagcagtggctgttggaaaagaagctaaactctacctgttccaagcccaggaatggctgaagctgctggagagcagccccggctacagctgcagtgagcggctagcccgagctcagctgacagtgacagtgacccagacggagcacaacctcacagtgtcccagctgcccgctccccagacatggcgagtgttctatgccgacaagttcacctgcagggatgactcagagagcccccagggggaggagatcccctttgaaatggtgctcctcaacccggacgccgagggaaacccactggatcattttagcgccagagagtccgggctccacgagttctttttcctcctcgtcctagtgtactttgtgactgcgtgcatctatgcgcagtctctgtggcaggctatgaagaagggaggacccatgcacaccatcttaaaggtcctcaccactgcactgctgcttcaagctgcttcagccttagctaattacatccacttgtccaggtactccagagatgggctaggagtgcctctcataggaagcctggcagaagtttttgacattgcctcccaaattcagatgctgtacctgcttctgagcctgtgtatgggctggacaatagtgcggatgaagaagtcgcagagcagaccgctccagtgggactcgacacccgcgtccacgggcatcgcagtcttcatcgtcatcacacagagcattttgctactctgggagcagtttgaagacaccagtcaccacagcgcacattcacaccgcagcttagccgggctcttgctgattgtcttacggatctgcctggcgctgtcgctgggctgcggactctaccaggtcatcacagtggagaggagcgcgctcaagagagagttctacatcacgtttgccaagggctgcatcctgtggttcttgtgccagccagcgctcgcatgcattgctgtcgcttttaatgactaccaaagagataagcttatcacagtaggtgtcatcctgtgtcaggccgtggccatggtcattctgtacagacttttcctgtcccacagtctttactgggaggtctcctcgctctcctcagtaacgctaccactgaccatctcgtctgcacacagagggcgccctcatttctgatgcttgagttttgtggagagaaccagtgaatggagaagtgcaataggatccaacgcagcaccgtcttgctgtgcctttgtgtgacagctgagcggtggaagcagggcgtcttatttatagaactgaacgtcagcgggctcagcagaaaggaatagaagctccggagtgaactcaaacagtgaacttcccagaaagaatgttgtttcaaggtgactgaaacagtttccacgtggaaataaatgtgaaaaggactgcttagagtacacgtgggccaggtggtcacacctgcgatgcctcgtcactagcaaactcaggcctgatagtcctacagtattcacctagacaatacttgcctgtgcgtgcccagctcgcccagttatgaaatcagcgggatgtgctgattttaaaactacttctttttatcctttaaagaacgtgcatttcaaattataatttaaaggacttgaaagtgaaattacttaggaaataaatagaaaatatgttaacagttaaacgaattagcatttgtttattcacgtgtggagttattctgcaatttctccttgtattgttggccctgcttcaggtcttatcttgattgaggaaggatcttggataaagtgtggaaaatcatgggtacattacattggttggaagaacagagagcttaagtgtgaggacgtgggtaagggacaagcagggttttgtgtttaaaaagaaagaaggaagaggtagtatgtggtatggccctgaaattttaatcctgtgataattcatttctcttgcgcggaatccccagtcctaatgaagctgtagacacagattaaattaacaggagcatttcacaggactgttagtttccgtcatcaacctacccacgaattcctgcccaaggatacttaatcaggattaaattttctatgaataattgaaaaacaaaatactcactggatttgttctaatccatgttggcactggatggtaaggagttgccatcttgaatcctttgtaataaagctgtgtgtgtgtgtgtttgtgtgtgtgtgtgtgtgtgtgtgtgtgtgtgtgtgtgtgtgtttgtgtgtgtgtgtgtgtgtgttgctccccaaattaagtatgctagtcctctaaaaggttcctcattaaagaaccactctgtagctgtcaggacttgctgtgtgctggtatctgtgaagtcgcttggaccttattgtttacttttaactaattttaaatatttaatctgcttgccatatttttaaacaggaaatggtgcaataatctggtaattgcgttttttaatggaagggttcagttaatgtagcagaatggaaaaggactgtggctttgtctttattttttccttgtctatagtttcattgaaaatgaaaatatcctgcttcattttaagacaaaatgtgtttgcagtcagcaaaatggcaaagtaatacagtctagcccgtgattaaaacatttctataggtt

SEQ ID NO: 11 - protein, 441 aa, Mus musculus
MGGLRLLAVALTCSCWWPQGGQGKTLRGSFSSAAARDAQGQSIGHFEFHGDHALLCVRINNVAVAVGKEAKLYLFQAQEWLKLLESSPGYSCSERLARAQLTVTVTQTEHNLTVSQLPAPQTWRVFYADKFTCRDDSESPQGEEIPFEMVLLNPDAEGNPLDHFSARESGLHEFFFLLVLVYFVTACIYAQSLWQAMKKGGPMHTILKVLTTALLLQAASALANYIHLSRYSRDGLGVPLIGSLAEVFDIASQIQMLYLLLSLCMGWTIVRMKKSQSRPLQWDSTPASTGIAVFIVITQSILLLWEQFEDTSHHSAHSHRSLAGLLLIVLRICLALSLGCGLYQVITVERSALKREFYITFAKGCILWFLCQPALACIAVAFNDYQRDKLITVGVILCQAVAMVILYRLFLSHSLYWEVSSLSSVTLPLTISSAHRGRPHF

SEQ ID NO: 12 - DNA, 8895 bp, Homo sapiens
ctcccccagctgccgacgtggggcgggcagccgccggcggctgggagccgaggcgtcggtgcagacctggagacgggcatgggggggctgcggctgctggctgtggccctcacgtgctgctggtggccgcagggcagccagggtaagaccctgcggggcagcttcagcagcaccgcggcccaggacgcccagggccagcgcatcggccacttcgagttccatggtgaccatgctcttctgtgtgtcagaatcaacaacatagcagtagctgttggaaaagaagctaaactctacctgttccaagcccaggaatggctaaagctacagcaaagcagtcatggttatagctgtagtgaaaaattatccaaagctcagttgacaatgaccatgaaccagaccgaacataatctgacagtgtcccagattccgtctccacaaacgtggcatgtgttttatgcagacaagtatacatgccaagatgacaaggagaattctcaggtggaagatatcccatttgaaatggtgttactaaacccagatgccgaagggaatccatttgatcattttagtgctggagaatctgggttacatgagttctttttcctcctagtcctagtgtactttgtgattgcttgcatttatgctcaatcattgtggcaggctattaagaaaggcggacccatgcacatgattttaaaggttctgacaactgcattgctgttacaagctggttcagctttagctaattacattcatttctccagttactccaaagatggaataggggtaccatttatgggaagtttggcagaattttttgacatcgcttcccaaattcagatgttatacttacttttgagtctatgcatgggttggacaatagtcagaatgaagaagtctcaaagcagacctctccagtgggattctacgcctgcatccactggcattgcagtattcattgtcatgacacagagtgttttgctactttgggaacagtttgaagatatcagtcatcatagctaccattcacaccacaacttagcagggatcctcctaattgttctaagaatttgcctagcattgtcattaggctgtggactctatcagatcatcacagtggagagaagtacactcaaaagggagttctacatcacatttgccaaaggctgtatcttgtggtttttatgccatccagttcttgcatgcatttctgtcatttttagcgactaccaaagagacaaggttattacaataggtgttatcctttgccagtctgtttccatggttattctctacagactctttctgtctcacagtctatactgggaagtttcttcactttcttcagtaacactaccactgaccatatcatctggacacaaaagtcgccctcatttctgatacttgatttttgttgagaggaaaagtgaattggttaaaagagtgcaataaggatccaaatacagtgacttttttttcatacatttagtatgaaaacttgaacagcgaaagcagagcatgttatttatataactgcatttaagcagtaccaagactgaaaaaaaaggtaataaatgaaatgttttgaaatatacttaaacaacaaactttgaagaaagtgttgttataaaattattgaagcgatttctatgtggaaataaatgtgaaaaataactatgatattttggtaaaatattcaccacttataatgcctcatcttaatagctaactcaggtttaatagtcttataaaaagtaatcagttaaatgattacttgcttataaatatctaaactagtccagttatgaaatcagtgtaatacattgatttttaaaactgctgctttttatgctttaaggaaaatgtatttcatatttgagtttaaaggaattgaaattacttcaggaaatgaatataaaataggttcacagttaaatgaataagcttttgtttatttgtgggtggagttattctccaattttttctgccatttttggctctagttcaggttttagcttgattagcaaaggtttttgacaaacagtttatgaaaaaataaaacttaaatacattacacgggttgtaaggacaaaggattttaaaatctgagcacttaggtgaagggacaagcaggtttatgtgtttaaacagaaagaagggaaaaggtactatgtgatatggtactgaaattttgatcccaatagaattcatttctcttacgttgaatccccaatcataattaagccgtatacacagattaaattaacagaagcatttcacataaatgttggtttcagtcatcaactacccatgaattcctgcccaaggatacttaatcaggattaaattttctatgaataattgaaaaacaaaacactcactggatttgttataatccgtgttggcactggattctaagtagttgccatcttgaatcctttgtaataaagccatacttttgtgtgtgtgtgtgatgccccagtattgagtatgctagctaaaaaatatcaagtgcctgattaaagaattgctaaatggttgtcaatactcgctgtataaaatgagtattcccattaatagtcactgattggaaattattctgttgctgtttgtcaagctgttcaggccttttcctttttacttctggcttattttaaaaatattttttatctatttactgtgtgtttaagcaagcaacagtgcactgatctggtagttaaattttttaattaaatagttaaaataatgtagtgcaacttaaaaatatctatggctttgtttttgtttttcttttgtacctagtctcattggaaatgactagtattctgcctcattttcagggcagaatatgtgtttgtgacccaaaatgacaaagttaccaagttcaacctgctcattaaatcatctctctattagttctttgtaaatcacttagtacattagagctacaggttaagactagaaagtctgagttatgttttaggtatacatgatcgtctgagtggtcataaccttttcttttatcatgtctcgctaataaccccagctattgtctgttgtgttcagctgagatgcaaaaaagaaattaagcaaaaaagaaaaagatgaagacttttcatgaattttgtgaacttgccaaaaggggaagggaaaaatctttgtgttacttcactcaaagaacataagtaactgcaattaacatatatgaaatttattactctgcttgcatttatgaaactaaccagttttttaaactttaattcttaaatttatggttggaaatgctgataatttattttgttatttaagagctgttgttaagtggaggaaagtagttgttaataaatgtataaaactgttcttgactagtaatcagggacaaaatttatagttcataagtcatgacacagtattcgctctttttctgaatgtttacatagagattcatcactgcagattacagaaagttaagtgtatactacaaactctaattaaagataaaaatcttttctacttttctttgtcagataatgcttttttatgtgtttttacattttttgaaagaagataaatggttcatccagagctttattaagaagcatttcttttcttcccttaaaaaatagatgcttatttttattgacatgtgtataataagatgggtgcctactgtgatgcattttaccaggcattttaccttcattattactcatctctattgtgataaccaggctcccatttaactgagtataagagagaataaatgatttctctaaggtcatcaaactatttagtttttccactttaccttcatgatttaaggaacatgtattagctgtgttttagtgagaatgaattgctatccatcataatttttgctgttgatttgaacattaaaactggaattgtgctaaaatcagaacatgcgcttgaatatctgtaagacctcagcacacttcagtatttgaacctggcttttaaccagacagtattactagcaggagagttaatctaggcagaagagcttgctttagggtaattatttattatctccagtaaagtcacatctgatgaacataggagatacacaaatctgagatcatctctaaaataatgagactagaacctatctatcaacatgaacatgggctccttcagcatgtttcttctaggagactgcacgttctttggaatttggaaatttggaattatctgtgaaacattttcttaaatagcttctgtggtatcaaatcattgaggttgttttttgtttgttttgagaattgatatgattgttagaagtagccagatacgaagaggtaatactgttttttattccaacatgtatttgtgaattgagactaaaataatgagattggttttcctctgtgatctgaaagtagtgtggtagaagcaccgtagcttgatttcttgcaatagctctctaagtaatattaccatgtccacacttgggcagccttctgcacagctccaggggcatcatatgtgttgtgctgtaggagtgccatgaagcctggaggtgaggaaagaagtgatcctgcccactagggccattctccacagtgtagccaaaacaatctcttaaaaatggaaatcatctggatgatgagcatacagaaattctttgtgctaatcttgtaatttttttgtaagtttgaaattatttcaaaaagtttaaaaatcatataaatcagatcaaactactacttccctgacaaaaaccctccatgtgcctctcattgcacttaggaaaagaagtcctcactcattactatgttctgcaaggcccatatgatctggttatctcttaatttgtaccactctctcacacacatatcccagcctcattgtccttcatcaaaaaaagatctagcttatatcacaccctcagatacttggcactagccatcttctctgcctgggacattctcttccaaatcttagcatggttggaaggctgaatcctcatccttcaggttctcagctgagatgtcttttagagaactcaccatctgtggtggatttatatggtgcagtttagtcaacctggagataggttctgcagaattctcttccatgcataatttcaggacaatgtaggccataagaaatgtttgatacaagatatagaaggcagaagtaaagtagcagccagatctgcattctttttgtaagaggaagggccagacaaggcacaaggcacaaggcactgttgcagcttatccacattattgcttatctgttggcttctcttggcctgtgggacaacagccaggcccacaggaacaccagttcctgccggaatggctccttcagcttctctggctcctgggccaggtgtgcgttttactctgacaaagggtgccagcttctgctggacaactccatcattcacactggaggattagaggcagggagagacctgtatggcttccagtccatcctcacaagttccactcagttcccacaggttctactcatcctagcaagttccttgcttcccccatttcccatgtcttccctttccaactgccagacacaaaagaaaccgcctcctataatccctataataagccccttattttgaatattaagatatgtgtatgtgtattagatcctagtggttcttttctgatcagaccctgattaaccataccatcctacctaatgctattcccccatcctagttctatcatattatccctttttttgtttgttgtacttagctggtttatctttgtcatctctcaaattagaatataaattttaagtgagtttgtctctattgttcattgctgtatgcctaatactaggacactccctggtgcatactagatgctcaaaaatatttttgagtgaatgaaggcaatgctgaaaaagttccaaatctgtacttaacagtgtagtattaattatgtaatagaagtacccgtagtgacttcaataataccttgcctattctttgttcaattgcatggaacttggtagtgtgtaaaaatcttgactagtcaagtagttgtcataaaacatcagttacgagaggatttctcaaattctcacagaggcagtttctctttattttatgtagtcctgtaataggaccatgtgtctggtttctggcctgccacaaccagaagcaccaatttaaaagagaagtttctagtcagctagaggagggaatgttgacagtagtcagaagcagctgggaaaagatgggaaaactagaaatgacaataacactcaaattgcagcagcctggggtcatttagtgtaggtgaaagcagccctgtgttgtgtatctcagtagactcttaagagatttgttgttcaatgtattcatgacagtgactgatggtaatcaagaaaatattaagttataagttatgttcagtagcctcaaagaagtatgcgaaaaccttctaatgttttcttcttgttaaacaggtaatatatgcacattgtacaaaaatcagaaagttgaaaaaggtggggaaaagaagtccttctctcactcatttcccttccctgaatccaaactctgttaccagttatctttctagaaatggccccatgcacatataaacatatatgaaacacaaattatagcataacatacagacttttgcacattcagcaatttattctgtagccttacctagaattgtcccatttttttcaccaattgcatagaactccattatatggatgtattttgattttttcaaagaatcagttctgttactgcacgttgttggttttcagtcttgctaatacagacaaggctgaaattaagacagtaatcaagaatttgccaagacttccttccctcctcccaaaatgcctccactccaggttccaggtctagatccttgggattttctaggtgatggaagtatctttgttattcatagtgagctctgatagttacgctaaccagatgactcaaataggggctagccacaccagaagaccaaccattggattagagggttggagttttgagtcatatgatattagcccaacctctgaggtggagagaggggttagagatggagttcagctttatatagagattgagtcaattaatcatgcctgtcatgaaaccccgagagaaactctggtcagtgatgctcaagtgaacttccctggttggcaatactctggtgtgttctaggagggtgacatgtccctgagggcacagaaactttgtgtttgggatcctcttacaccttgctctatgtatgtcttcttttggctgttctgatttgtatccttttgctataataaactgtaatagtacgcatagcactttgctgagttctgtgagttgttctatttaattatagaacctgaaggtattgtggggacccctgaacttgcagccagttggtcagaagtaaggatggtcctgggaatgcccaaagttgctagtttctgaagtgagggcagtcatatggaggactgggcccttaatctgtgaagtttggcttaacttcaggcagttggtgtcagaagccattgcaaatatcctgatgatccgtgcaaaacaaaaacacttaacagaaaattacgttgtactctctgtacggataattttcggttaagtgtttttgttttccatggattgtactagatatcaatctgataaataacttttattcattatagttttaatttgcatttctcttattaatttttgttttaaaaccattttctttccagttctacgatctgttaacatatttttcccacttttctattgggttggttgtctttttcttattgatttgttaaaactctttataacagaaattggtcatttgtgatatgagtctgaactgtgtttctcaatttgtgatttttctctttacttttttaatggtgtctttttcttgtagtttttttaatgcaggtggaattatcagtctttcatttttttcttctgtgttctacctgtttcacaggtagaaaagccttagctctttgagattatgtttaaatgccctgtattttcatctagtggttttatggttttattttgtatgtttttttcatccacctggaatttattttgctgttaaaatatgaattagagatccaacttaatttttttcagatggttgttgacttattgcaatatcatgtgttatataatccatctttactgattcgttgctaattggtttctggacttttaaggctatctgatgaactgttgatctggtaccacactgtttcaattattgtagtcttaaaattgctgtacgctttggtagggataattttctgttaagtgtttttgttttttataattttccaattagtcttgctcattttttatataaactttgaaatcaatttttctgattttcaaaagaaaaaaaatcctgtttagtattcttagtggaatgaattaaatttccatagcaatttagggaaatttgactttcttaacgtgttgagtgttaaacccacaggtggtagcatgacattgactcctcagccaagcatattatcaactgtcggaaactaggttttcccatagtgatccaagaaactagtacgaaatgcctttctgattttttaaaacttgtatacatatatagtacatattatatatacttatatattaagtaatcagaagtatgtgtttctcaatcaccttttaaagacttctttatatagctcttgtacatttcttgatatgaatatgttaatgttttctttcattatgttgcatatggcttttgttttctgcatgaaaactattgatttctgcattctaattttgttcccagtcacctaactgaattcttctaacagttgtaataaatttctggttggctctcttgtgaaaaaaaaaaaaaaaaaaaa

SEQ ID NO: 13 - protein, 440 aa, Homo sapiens
MGGLRLLAVALTCCWWPQGSQGKTLRGSFSSTAAQDAQGQRIGHFEFHGDHALLCVRINNIAVAVGKEAKLYLFQAQEWLKLQQSSHGYSCSEKLSKAQLTMTMNQTEHNLTVSQIPSPQTWHVFYADKYTCQDDKENSQVEDIPFEMVLLNPDAEGNPFDHFSAGESGLHEFFFLLVLVYFVIACIYAQSLWQAIKKGGPMHMILKVLTTALLLQAGSALANYIHFSSYSKDGIGVPFMGSLAEFFDIASQIQMLYLLLSLCMGWTIVRMKKSQSRPLQWDSTPASTGIAVFIVMTQSVLLLWEQFEDISHHSYHSHHNLAGILLIVLRICLALSLGCGLYQIITVERSTLKREFYITFAKGCILWFLCHPVLACISVIFSDYQRDKVITIGVILCQSVSMVILYRLFLSHSLYWEVSSLSSVTLPLTISSGHKSRPHF

SEQ ID NO: 14 - protein, 296 aa, Homo sapiens
PFEMVLLNPDAEGNPFDHFSAGESGLHEFFFLLVLVYFVIACIYAQSLWQAIKKGGPMHMILKVLTTALLLQAGSALANYIHFSSYSKDGIGVPFMGSLAEFFDIASQIQMLYLLLSLCMGWTIVRMKKSQSRPLQWDSTPASTGIAVFIVMTQSVLLLWEQFEDISHHSYHSHHNLAGILLIVLRICLALSLGCGLYQIITVERSTLKREFYITFAKGCILWFLCHPVLACISVIFSDYQRDKVITIGVILCQSVSMVILYRLFLSHSLYWEVSSLSSVTLPLTISSGHKSRPHF